SEQUENCE LISTING (56 sequences)

SEQ ID NO: 1 - DNA, 18 bp, synthetic construct
  source 1..18: mol_type = other DNA
cactgtcgagtcgcgtcc

SEQ ID NO: 2 - DNA, 20 bp, synthetic construct
  source 1..20: mol_type = other DNA
tcatccatggcgaactggtg

SEQ ID NO: 3 - DNA, 22 bp, synthetic construct
  source 1..22: mol_type = other DNA
gtccccaaagggatgagaagtt

SEQ ID NO: 4 - DNA, 20 bp, synthetic construct
  source 1..20: mol_type = other DNA
ctcctccacttggtggtttg

SEQ ID NO: 5 - DNA, 21 bp, synthetic construct
  source 1..21: mol_type = other DNA
cccatgatctggaagagacca

SEQ ID NO: 6 - DNA, 20 bp, synthetic construct
  source 1..20: mol_type = other DNA
caaacttctgcctgacgagc

SEQ ID NO: 7 - DNA, 21 bp, synthetic construct
  source 1..21: mol_type = other DNA
tgccaccttttgacagtgatg

SEQ ID NO: 8 - DNA, 20 bp, synthetic construct
  source 1..20: mol_type = other DNA
tgatgtgctgctgcgagatt

SEQ ID NO: 9 - DNA, 22 bp, synthetic construct
  source 1..22: mol_type = other DNA
acgagagttgcctggctactag

SEQ ID NO: 10 - DNA, 23 bp, synthetic construct
  source 1..23: mol_type = other DNA
cctcatagatgctaccaaggcac

SEQ ID NO: 11 - DNA, 20 bp, synthetic construct
  source 1..20: mol_type = other DNA
agaccctgcccattgaactg

SEQ ID NO: 12 - DNA, 20 bp, synthetic construct
  source 1..20: mol_type = other DNA
ggcgggtctggtttgatgat

SEQ ID NO: 13 - DNA, 20 bp, synthetic construct
  source 1..20: mol_type = other DNA
caccatatggctcggacacc

SEQ ID NO: 14 - DNA, 20 bp, synthetic construct
  source 1..20: mol_type = other DNA
tcgagtgacaaacacgactg

SEQ ID NO: 15 - DNA, 21 bp, synthetic construct
  source 1..21: mol_type = other DNA
ccacgtgttgagatcattgcc

SEQ ID NO: 16 - DNA, 20 bp, synthetic construct
  source 1..20: mol_type = other DNA
tcactccagttaaggagccc

SEQ ID NO: 17 - DNA, 20 bp, synthetic construct
  source 1..20: mol_type = other DNA
cggccttccctacttcacaa

SEQ ID NO: 18 - DNA, 21 bp, synthetic construct
  source 1..21: mol_type = other DNA
tgccattgcacaactcttttc

SEQ ID NO: 19 - DNA, 20 bp, synthetic construct
  source 1..20: mol_type = other DNA
cactcacctgctgctactca

SEQ ID NO: 20 - DNA, 22 bp, synthetic construct
  source 1..22: mol_type = other DNA
gcttggtgacaaaaactacagc

SEQ ID NO: 21 - DNA, 20 bp, synthetic construct
  source 1..20: mol_type = other DNA
tgtccacagctttgctgaat

SEQ ID NO: 22 - DNA, 21 bp, synthetic construct
  source 1..21: mol_type = other DNA
gaagtgtggtagcgaggaagc

SEQ ID NO: 23 - DNA, 20 bp, synthetic construct
  source 1..20: mol_type = other DNA
cgtgggagatgtcctcaact

SEQ ID NO: 24 - DNA, 20 bp, synthetic construct
  source 1..20: mol_type = other DNA
agatctctgctcggaccacc

SEQ ID NO: 25 - DNA, 21 bp, synthetic construct
  source 1..21: mol_type = other DNA
acagcagcgtggagagatatg

SEQ ID NO: 26 - DNA, 20 bp, synthetic construct
  source 1..20: mol_type = other DNA
gttaagccggttagtcccgt

SEQ ID NO: 27 - DNA, 20 bp, synthetic construct
  source 1..20: mol_type = other DNA
tcacccggaaatacacgctc

SEQ ID NO: 28 - DNA, 20 bp, synthetic construct
  source 1..20: mol_type = other DNA
ggcctcgaaagtaaccggaa

SEQ ID NO: 29 - DNA, 21 bp, synthetic construct
  source 1..21: mol_type = other DNA
gctgtcatcgatttctcccct

SEQ ID NO: 30 - DNA, 23 bp, synthetic construct
  source 1..23: mol_type = other DNA
gacaccttggtcttggagcttat

SEQ ID NO: 31 - DNA, 21 bp, synthetic construct
  source 1..21: mol_type = other DNA
tccctgcatagaggtagttcc

SEQ ID NO: 32 - DNA, 20 bp, synthetic construct
  source 1..20: mol_type = other DNA
tcaaggggttgaagctcaga

SEQ ID NO: 33 - DNA, 21 bp, synthetic construct
  source 1..21: mol_type = other DNA
tgtccaacaatggcatcacct

SEQ ID NO: 34 - DNA, 20 bp, synthetic construct
  source 1..20: mol_type = other DNA
tgtgttccctcgaagccaaa

SEQ ID NO: 35 - DNA, 17 bp, synthetic construct
  source 1..17: mol_type = other DNA
ctcgcctttgccgatcc

SEQ ID NO: 36 - DNA, 20 bp, synthetic construct
  source 1..20: mol_type = other DNA
tctccatgtcgtcccagttg

SEQ ID NO: 37 - DNA, 20 bp, synthetic construct
  source 1..20: mol_type = other DNA
tgggatcattgccctgtgag

SEQ ID NO: 38 - DNA, 20 bp, synthetic construct
  source 1..20: mol_type = other DNA
ggtgtctgaaggagggggta

SEQ ID NO: 39 - DNA, 20 bp, synthetic construct
  source 1..20: mol_type = other DNA
accggaaggaaccatctcac

SEQ ID NO: 40 - DNA, 21 bp, synthetic construct
  source 1..21: mol_type = other DNA
ggcaaaactgcaccttcacac

SEQ ID NO: 41 - DNA, 33 bp, synthetic construct
  source 1..33: mol_type = other DNA
atcttgggagaatttgctgatgaaaggaatctg

SEQ ID NO: 42 - DNA, 33 bp, synthetic construct
  source 1..33: mol_type = other DNA
caaccgatggacgtgtaaaccagccaggttttg

SEQ ID NO: 43 - DNA, 30 bp, synthetic construct
  source 1..30: mol_type = other DNA
cattagatgtgctcttaagcagtttccaac

SEQ ID NO: 44 - DNA, 31 bp, synthetic construct
  source 1..31: mol_type = other DNA
attgataaggattgccatttgaaatgtttag

SEQ ID NO: 45 - DNA, 30 bp, synthetic construct
  source 1..30: mol_type = other DNA
catcaataccagcttctttcgagggaatac

SEQ ID NO: 46 - DNA, 30 bp, synthetic construct
  source 1..30: mol_type = other DNA
tattcacagtctctcctttcagagctctgc

SEQ ID NO: 47 - DNA, 31 bp, synthetic construct
  source 1..31: mol_type = other DNA
cgagggaatactagctcctaagggccattac

SEQ ID NO: 48 - DNA, 30 bp, synthetic construct
  source 1..30: mol_type = other DNA
aaagagaatggtattgatgtattcacagtc

SEQ ID NO: 49 - DNA, 33 bp, synthetic construct; identical to SEQ ID NO: 41
  source 1..33: mol_type = other DNA

SEQ ID NO: 50 - DNA, 33 bp, synthetic construct; identical to SEQ ID NO: 42
  source 1..33: mol_type = other DNA

SEQ ID NO: 51 - DNA, 30 bp, synthetic construct; identical to SEQ ID NO: 43
  source 1..30: mol_type = other DNA

SEQ ID NO: 52 - DNA, 31 bp, synthetic construct; identical to SEQ ID NO: 44
  source 1..31: mol_type = other DNA

SEQ ID NO: 53 - DNA, 30 bp, synthetic construct; identical to SEQ ID NO: 45
  source 1..30: mol_type = other DNA

SEQ ID NO: 54 - DNA, 30 bp, synthetic construct; identical to SEQ ID NO: 46
  source 1..30: mol_type = other DNA

SEQ ID NO: 55 - DNA, 31 bp, synthetic construct; identical to SEQ ID NO: 47
  source 1..31: mol_type = other DNA

SEQ ID NO: 56 - DNA, 30 bp, synthetic construct; identical to SEQ ID NO: 48
  source 1..30: mol_type = other DNA

(19) United States
(12) Patent Application Publication
(10) Pub. No.: US 2026/0048073 A1
(43) Pub. Date: Feb. 19, 2026

(54) TOLL-LIKE RECEPTOR 4 (TLR4) AGONIST AND METHODS OF USE THEREOF

(71) Applicant: Nanyang Technological University, Singapore (SG)

(72) Inventors: Yuan QIAO, Singapore (SG); Chenyu LI, Singapore (SG); Christopher William Emmanuel ADAMSON, Singapore (SG)

(21) Appl. No.: 19/300,420

(22) Filed: Aug. 14, 2025

(30) Foreign Application Priority Data
Aug. 14, 2024 (SG) 10202402453U
Apr. 17, 2025 (SG) 10202501012Y

(51) Int. Cl. A61K 31/7016; A61P 1/04; C07H 3/04

(52) U.S. Cl. CPC A61K 31/7016; A61P 1/04; C07H 3/04

(57) ABSTRACT

The present invention relates generally to the field of immunomodulation and more specifically to compounds that exhibit Toll-like receptor 4 (TLR4) agonist activity. The invention provides novel compounds capable of stimulating or promoting an immune response, thereby offering potential therapeutic applications. In particular, the compounds may be useful in methods of promoting and/or maintain gastrointestinal health, as well as treating or preventing gastrointestinal diseases, such as IBD and colitis.

### CROSS-REFERENCE TO RELATED APPLICATION

This application claims the benefit of priority of Singapore Patent Application No. 10202402453U filed 14 Aug. 2024 and Singapore Patent Application No. 10202501012Y filed 17 Apr. 2025, the content of which is hereby incorporated by reference in its entirety for all purposes.

### REFERENCE TO AN ELECTRONIC SEQUENCE LISTING

The contents of the electronic sequence listing (P130032-Sequence Listing.xml; Size: 50,294 bytes; and Date of Creation: Apr. 9, 2025) are herein incorporated by reference in their entirety.

### FIELD OF THE INVENTION

Various embodiments relate generally to the field of immunomodulation and more specifically to compounds that exhibit Toll-like receptor 4 (TLR4) agonist activity. The invention provides novel compounds capable of stimulating or promoting a TLR4-mediated immune response, thereby offering potential therapeutic applications. In particular, via their TLR4 agonist activity, the compounds may be useful in promoting and/or maintaining gastrointestinal health, as well as treating or preventing gastrointestinal diseases, such as IBD and colitis.

### BACKGROUND

The gut microbiota has a profound impact on host health. The resident gut bacteria bestow a rich source of microbial metabolites and molecules, known as microbe-associated molecular patterns (MAMPs), which are recognized by the host's innate immune pattern recognition receptors (PRRs). [1] Proper interactions between commensal bacterial MAMPs and host PRRs are critical for intestinal homeostasis, including the genesis and maturation of isolated lymphoid follicles (ILFs), [2] reinforcement of gut barrier functions, [3] and protection against colonic injury and pathogenic infections. [4] Conversely, gut microbiota dysbiosis can inflict dysregulated immune responses in hosts, leading to chronic inflammatory diseases such as inflammatory bowel diseases (IBD), rheumatoid arthritis, and asthma. [5] Understanding how the host recognizes and responds to gut microbial ligands is a key step toward developing novel immuno-therapeutics to target the gut microbiota-host interface.

Peptidoglycan, the major bacterial cell wall component, represents a well-known MAMP that stimulates the host's innate immune system. [6] A mesh-like layer that surrounds the bacterial cytoplasmic membrane, peptidoglycan is composed of repeating N-acetylglucosamine-β-1,4-N-acetylmuramic acid (GlcNAc-MurNAc) disaccharide with a stem peptide connected to the lactoyl group of each MurNAc unit. Adjacent peptidoglycan strands are cross-linked via the appended stem peptides. [7] Despite a conserved scaffold, peptidoglycan varies significantly across bacterial species in terms of stem peptide composition, type and degree of crosslinking, and unique glycan modifications, giving rise to remarkably complex and heterogeneous peptidoglycan polymeric structures (i.e. peptidoglycome). [8] During bacterial cell wall remodelling and turnover, soluble peptidoglycan fragments (PGNs) are generated and released into the milieu. These PGN fragments can activate mammalian NOD1/2 innate immune receptors, leading to the production of proinflammatory cytokines. [9] Notably, NOD1 and NOD2 recognize distinct minimal PGN motifs: NOD1 detects the dipeptide D-γ-Glu-mDAP (iE-DAP),[10] while NOD2 recognizes the muramyl dipeptide N-acetylmuramyl-L-Ala-D-isoGln (MDP or M-AQ). [11] Biological studies of bacterial PGNs have predominantly focused on these canonical NOD1/2 ligands. Given the diversity of the gut microbiota peptidoglycome, these ligands likely cannot embody the repertoire of natural gut microbiota-derived PGNs in hosts, which may exhibit bioactivity via NOD-independent pathways. For instance, GlcNAc derived from peptidoglycan was shown to trigger NLRP3 inflammasome formation by inhibiting the metabolic enzyme hexokinase in primed immune cells without engaging NOD1/2. [12] Moreover, an anti-inflammatory 1,6-anhydro-PGN motif from probiotic Bifidobacterium was recently identified to not activate NOD1/2, [13] suggesting alternate PGN sensing mechanisms in hosts. However, despite increasing recognition of gut microbiota-derived PGNs as key effector molecules in hosts, the fundamental questions regarding the structures of natural PGNs, their biological roles, and host responses beyond NOD1/2 signalling are yet to be determined.

Traditionally, detection of PGNs has largely relied on cell-based reporter assays that selectively respond to NOD1/2 ligands but not non-canonical motifs. [14] A monoclonal antibody (mAb) 2E7 has been developed that specifically recognizes MDP, which enabled an indirect competitive enzyme-linked immunosorbent assay (icELISA) for PGN detection. This advancement has led to the discovery of gut microbiota-derived PGNs in host systemic circulation; however, the exact structures of natural PGNs remain unknown. [15]

Accordingly, there is still a need in the art to investigate gut microbiota-derived PGNs to fully understand their biological functions in hosts and consequently lead to the discovery and development of novel compounds capable of modulating an immune response, especially within the gastrointestinal tract of subjects, with potential utility in promoting and/or maintaining gut health and homeostasis, thereby offering therapeutic and preventive benefits for a range of diseases and conditions, as well as non-therapeutic applications.

### SUMMARY

Various embodiments meet this need by in one aspect providing a Toll-Like Receptor 4 (TLR4) agonist having a formula (I):

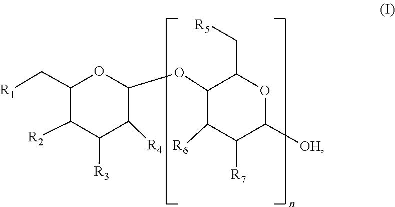

wherein
n is an integer number selected from 1-4, and
R₁, R₂, R₃, R₄, R₅, R₆, R₇ independently selected from the group consisting of —H, —OH, —NHC(O)Me, —OC(CH₃)COOH, —NH₂, and —OP(O)(OH)₂,
or a pharmaceutically acceptable salt, stereoisomeric form, or regioisomeric form thereof.

In various embodiments, one or both of R₄ and R₇ are —NHC(O)Me.

In various embodiments, n is 1.

In various embodiments, the TLR4 agonist has a formula (II):

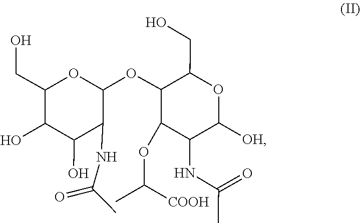 [SMILES: CC(=O)NC1C(OC2C(CO)OC(O)C(NC(C)=O)C2OC(C)C(=O)O)OC(CO)C(O)C1O]

or a pharmaceutically acceptable salt or regioisomeric form thereof.

In various embodiments, the regioisomeric form of the TLR4 agonist has the formula (III):

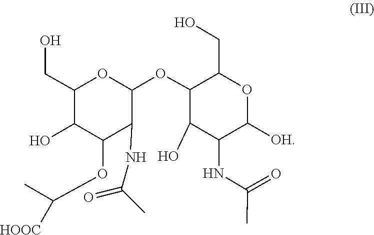 [SMILES: CC(=O)NC1C(O)OC(CO)C(OC2OC(CO)C(O)C(OC(C)C(=O)O)C2NC(C)=O)C1O]

In various embodiments, the TLR4 agonist has a formula (II):

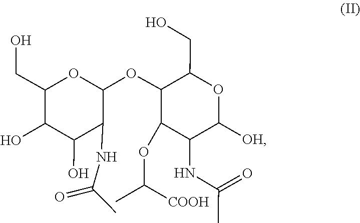 [SMILES: CC(=O)NC1C(OC2C(CO)OC(O)C(NC(C)=O)C2OC(C)C(=O)O)OC(CO)C(O)C1O]

In various embodiments, the TLR4 agonist is free of any attached peptide (e.g. stem peptide).

In various embodiments, the TLR4 agonist is synthetically derived.

In another aspect, there is provided a composition comprising the Toll-Like Receptor 4 (TLR4) agonist disclosed herein.

In various embodiments, the composition is a nutraceutical composition, or a supplement composition or a nutritional composition.

In various embodiments, the composition is a pharmaceutical composition.

In various embodiments, the Toll-Like Receptor 4 (TLR4) agonist disclosed herein or composition disclosed herein, is for use as a medicament.

In various embodiments, the Toll-Like Receptor 4 (TLR4) agonist disclosed herein or composition disclosed herein, is for use as an adjuvant.

In another aspect, there is provided a postbiotic comprising the Toll-Like Receptor 4 (TLR4) agonist disclosed herein or composition disclosed herein.

In another aspect, there is provided an engineered probiotic microorganism capable of producing and secreting the Toll-Like Receptor 4 (TLR4) agonist disclosed herein.

In another aspect, there is provided a use of the Toll-Like Receptor 4 (TLR4) agonist disclosed herein or composition disclosed herein, in the manufacture of a medicament for promoting or maintaining gastrointestinal health in a subject; and/or modulating gut microbiota in a subject.

In another aspect, there is provided a non-therapeutic method for promoting or maintaining gastrointestinal health, or modulating gut microbiota, in a subject, comprising administering the Toll-Like Receptor 4 (TLR4) agonist disclosed herein or composition disclosed herein, or postbiotic disclosed herein, or engineered probiotic microorganism disclosed herein to the subject.

In various embodiments, the subject is a healthy subject.

In another aspect, there is provided a use of the Toll-Like Receptor 4 (TLR4) agonist disclosed herein or composition disclosed herein, in the manufacture of a medicament for stimulating or eliciting an immune response in a subject.

In another aspect, there is provided a use of the Toll-Like Receptor 4 (TLR4) agonist disclosed herein or composition disclosed herein, in the manufacture of a medicament for treating, preventing, or ameliorating a gastrointestinal disease, preferably the gastrointestinal disease is an inflammatory bowel disease (IBD).

In various embodiments, the IBD is colitis.

### BRIEF DESCRIPTION OF THE DRAWINGS

Various embodiments will be better understood with reference to the detailed description when considered in conjunction with the non-limiting examples and the accompanying drawings.

FIG. 1A-1D shows that HPLC-MS/MS analysis of muramic acid (MurN) enables global quantification of gut microbiota-derived peptidoglycan fragments (PGNs) in biological samples: FIG. 1A Scheme of sample preparation for MurN analysis ([M+H]+: 252.1078); FIG. 1B Extracted ion chromatograms (EICs) of MurN detected in host samples compared to the MurN standard; FIG. 1C MS/MS spectra of MurN detected in host samples compared to the MurN standard; and FIG. 1D MurN concentrations in biological samples, including SPF mice ceca (n=4) and feces (n=25), GF mice feces (n=8), human stools (n=12) and sera (n=11), and commercial fetal bovine serum (FBS) from two different brands. Data are presented as mean values±s.e.m. (n as indicated). Statistical significance was determined using one-way ANOVA.

FIG. 2A-2G shows the quantification of MurN derived from PGNs by HPLC-HRMS/MS: FIG. 2A Acid hydrolysis of N-acetyl muramic acid (MurNAc) standard yields muramic acid (MurN) ([M+H]+: 252.1078); FIG. 2B MS/MS spectrum is shown with the two prominent fragment peaks ([M+H]+: 126.0549 and 144.0655) selected for quantification; FIG. 2C-2E The MurN calibration curve was established with the parallel reaction monitoring (PRM) mode of HPLC-HRMS/MS; FIG. 2C extracted ion chromatograms (EICs) of two selected MS/MS fragments of the precursor MurN with the area under curves (AUCs) calculated; FIG. 2D EICs of the signature fragment ([M+H]+: 126.0549) of MurN derived from serial dilutions of MurNAc; FIG. 2E calibration curve generated by plotting of the AUCs of the signature fragments against known concentrations of MurNAc gives rise to the standard calibration curve. Data are presented as mean values±s.d. of technical replicates (n=3); FIG. 2F Evaluations of sample matrix effects. Each pooled biological sample (stool or sera) was spiked with a series of known concentrations of MurNAc, followed by hydrolysis and MurN analysis; and FIG. 2G Representative plots for mice feces and human sera are shown, where the suppression effects are derived from the slope of the respective plots, where the observed MurN concentrations in biological samples were plotted against the known spiked-in MurNAc concentrations. Data are presented as mean values±s.d. of technical replicates (n=3).

FIG. 3A-3D shows the profiling of gut microbiota-derived PGNs reveals abundant saccharide moieties in the host gut: FIG. 3A Scheme of HPLC-HRMS/MS workflow for PGN identification; FIG. 3B ElCs of major PGN subtypes in the host feces: ahM, M, GM, and M-AE; FIG. 3C chemical structures of ahM, M, GM, and M-AE; and FIG. 3D Chemical synthetic route of disaccharide GM.

FIG. 4A-4C shows the profiling of natural PGN subtypes in the host gut: FIG. 4A The list of natural PGN subtypes found in mice feces and healthy human stools, with the corresponding structural formulae, exact masses ([M+H]+), and approximated concentrations are shown. N.d. stands for not detected; FIG. 4B Pie charts showing the relative abundance of detectable PGN subtypes in mouse feces and human stools. The saccharide-only moieties account for nearly 90% of the soluble PGNs in host feces; and FIG. 4C Profiling of gut microbiota-derived PGNs in feces and ceca of individual mice reveals distinct PGN compositions along the GI tract (n=5). G: N-acetylglucosamine; M: N-acetylmuramic acid; ahM: 1,6-anhydro-N-acetylmuramic acid; DG: di-acetylglucosamine; DM: di-acetylmuramic acid; UG: glucosamine; m: meso-diaminopimelic acid.

FIG. 5A-5C shows LC-MS/MS confirmation of two major PGN subtypes MurNAc-AE (M-AE) and GlcNAc-MurNAc (GM) found in human stools: FIG. 5A Chemical structure of M-AE ([M+H]+: 494.1980) and MS/MS spectrum detected in human stool; FIG. 5B Chemical structure of GM ([M+H]+: 497.1977) and MS/MS spectrum detected in human stool; and FIG. 5C MS/MS spectra of the synthetic GM and MG disaccharides, which are regio-isomers. Comparisons of the relative abundance of two MS/MS fragments ([M+H]+: 204.0867 and 276.1078) in synthetic standards indicate that the natural isomer found in human stool is GM.

FIG. 6A-6I shows the LC-MS/MS validation of PGN monosaccharides M and ahM found in host sera: FIG. 6A EICs of M and ahM detected in human sera and fetal bovine serum (FBS); FIG. 6B EIC of human serum showing the chromatographic peak corresponding to M at m/z 294.1182; FIG. 6C MS1 mass spectrum of human serum highlighting the ion at m/z 294.1182 among other detected ions; FIG. 6D Expanded isotopic distribution of the ion at m/z 294.1182 detected in human serum (upper) compared with the isotopic distribution of the synthetic standard (lower); FIG. 6E MS/MS fragmentation spectrum of compound M in human serum (upper) compared with the MS/MS fragmentation spectrum of the synthetic standard (lower); FIG. 6F EIC of human serum showing the chromatographic peak corresponding to compound ahM at m/z 276.1078; FIG. 6G MS1 mass spectrum of human serum highlighting the ion at m/z 276.1078 among other detected ions; FIG. 6H Expanded isotopic distribution of the ion at m/z 276.1078 detected in human serum (upper) compared with the isotopic distribution of the synthetic standard (lower); FIG. 6I MS/MS fragmentation spectrum of compound ahM in human serum (upper) compared with the MS/MS fragmentation spectrum of the synthetic standard (lower).

FIG. 7A-7I shows a transwell assay to evaluate the translocation of saccharides (M and GM) and muropeptide (M-AEKAA): FIG. 7A Chemical structures of M, GM, and M-AEKAA used in the translocation assay. The saccharides M and GM translocate faster than the larger muropeptide M AEKAA; FIG. 7B The transwell assay setup, aliquots of samples were collected from the well at the indicated time points (0, 1, 2, 3, 5, 16 h) for LC-MS quantification; FIG. 7C a line graph showing LC-MS quantification of PGN subtypes GM, M, and M-AEmAA over time. The y-axis represents the area under the curve (AUC), and the x-axis represents the time of sample collection (0, 2, 4, 8, and 16 h). Data are presented as mean values±s.e.m. (n=5); FIG. 7D a bar graph showing quantification of FITC-dextran fluorescence intensity in samples collected at 1 h and 36 h from wells with or without Caco-2 cells. The y-axis represents fluorescence intensity. Data are presented as mean values±s.e.m. (n=3-15); FIG. 7E schematic representation of the structure of M-AEKAA (m/z 764.3672) and its degradation into amidase-mediated degradation products, M ([M+H]+: 294.1183) and AEKAA ([M+H]+: 489.2667) following incubation with fetal bovine serum (FBS); FIG. 7F chromatograms show that incubation of M-AEKAA with 10% FBS resulted in the detection of intact M-AEKAA as well as the degradation products M and AEKAA; FIG. 7G chromatograms show that incubation of M-AEKAA with heat-quenched 10% FBS resulted in detection of intact M-AEKAA, while the degradation products M and AEKAA were not detectable. Heat-inactivated FBS did not have the cleavage effects; FIG. 7H MS/MS fragmentation spectrum of synthetic muramic acid (M); and FIG. 7I MS/MS fragmentation spectrum of muramic acid (M) generated by FBS-mediated cleavage of M-AEKAA, aligning with the fragmentation pattern of the synthetic standard. The experiments were repeated at least twice with similar results obtained.

FIG. 8A-8L shows the disaccharide GM exhibits immuno-stimulatory effects in vitro independent of NOD1/2 receptors: FIG. 8A SEAP reporter readouts from HEK-Blue™ NOD1/2 reporter cells stimulated with MDP (200 nM), M-AE (200 nM), GM (20 μM), or M (20 μM) for 18 h. Data are presented as mean values of biological replicates (n=3); FIG. 8B PGNs induce cytokine production in macrophages and monocytes and activate dendritic cells. The respective immune cells were stimulated with the indicated PGNs for 24 h followed by cytokine analysis by RT-qPCR (FIG. 8C-8F) or ELISA (FIG. 8G-8H); FIG. 8C bar graph showing relative Tnfα expression in RAW264.7 cells treated with GM (4 mM, 2 mM, 1 mM), M (4 mM), or MDP (0.4 mM); FIG. 8D bar graph showing relative Tnfα expression in murine bone marrow derived macrophages (BMDMs) cells treated with GM (4 mM, 2 mM, 1 mM), M (4 mM), or MDP (0.4 mM); FIG. 8E bar graph showing relative Il1β expression in BMDMs cells treated with GM (8 mM, 4 mM, 2 mM), M (8 mM) or MDP (0.4 mM); FIG. 8F bar graph showing relative Tnfα expression in THP-1 cells treated with GM (8 mM, 4 mM, 2 mM), M (8 mM) or MDP (0.4 mM); Data are presented as mean values±s.e.m. of biological replicates (n=3-4). Statistical significance was calculated using one-way ANOVA; FIG. 8G bar graph showing IL-6 concentration (pg/mL) in supernatants of BMDMs treated with GM (8 mM, 4 mM, 2 mM), M (8 mM) or MDP (0.4 mM); FIG. 8H bar graph showing IL-8 concentration (pg/mL) in supernatants of THP-1 cells treated with GM (8 mM, 4 mM, 2 mM), M (8 mM) or MDP (0.4 mM); FIG. 8I shows the mean fluorescent intensity (MFI) of surface CD80/86 in cDC2s population of murine bone marrow-derived dendritic cells (BMDMs) that were treated with MDP (20 μM) or GM (50 μM, 100 μM, 200 μM) for 18 h. Data are presented as mean values±s.e.m. of biological replicates (n=2-3). Statistical significance was calculated using two-way ANOVA; FIG. 8J-8K Assays using polymyxin B (PB) confirmed that GM was not contaminated with trace endotoxin; FIG. 8J ELISA analysis of the TNFα levels in BMDMs stimulated with GM (4 mM), LPS (100 ng/ml), or polymyxin B resin (PB-resin)-neutralized GM (4 mM) or LPS (100 ng/ml) for 24 h. Data are presented as mean values±s.e.m. of biological replicates (n=3). Statistical significance was calculated using one-way ANOVA; FIG. 8K The TLR4 inhibitor specifically suppresses the GM-induced TNF-a production. The RAW_Dual™ cells were pre-incubated with the respective TLR inhibitors (10 μM) for 1 h followed by GM stimulation (2 mM) for 24 h. TLR2i, TLR4i, and TLR7/9i refer to CU_CPT22, TAK-242, and AT791, respectively. Data are presented as mean values±s.e.m. of biological replicates (n=3). Statistical significance was determined using one-way ANOVA; and FIG. 8L SEAP reporter readouts from HEK-Blue™ mTLR4 reporter cells stimulated with GM (1 mM) or LPS (100 ng/ml) titrated with polymyxin B (0, 0.1, 1, 10 kU) for 24 h. Data are presented as mean values±s.e.m. of biological replicates (n=3). Statistical significance was calculated using two-way ANOVA.

FIG. 9A-9B shows the disaccharide GM activates murine bone marrow-derived dendritic cells (BMDCs): FIG. 9A Gating strategies used for analysis; and FIG. 9B Representative histograms of surface marker CD80/86 expressions in the cDC2 population of BMDCs treated with GM or MDP (control) at the indicated concentrations for 16 h.

FIG. 10A-10B shows the validation of synthetic PGNs is endotoxin-free by LAL assay: FIG. 10A LPS (1000 ng/mL), LPS neutralized by Polymyxin B agarose beads (PB), GM (1 mM), MDP (1 mM), and M-AE (1 mM) were tested according to the manufacturer's recommended protocol; and FIG. 10B Plate image of polymyxin B titration assay in HEK-Blue™ mTLR4 reporter cells as described in FIG. 8J.

FIG. 11 shows that no degradation products of the disaccharide GM were detected in GM-treated HEK cells by LC-MS analysis. HEK-Blue™ cells were incubated with GM (0.4 mM) for 24 h, followed by cell lysis and LC-MS analysis. EICs of the target ions: GM ([M+H]+: 497.19772), potential degradation products G ([M+H]+: 222.09721) and M ([M+H]+: 294.11834), were shown. The disaccharide GM retains its disaccharide structural integrity in host cells.

FIG. 12 shows the validation of TLR inhibitors to suppress the pro-inflammatory effects of their respective canonical ligands. RAW_Dual™ cells were preincubated with various TLR inhibitors (i.e., TLR2i, TLR4i, and TLR7/9i, same as the ones shown in FIG. 8L) for 1 h, followed by the addition of canonical TLR ligands for 24 h. The canonical TLR ligands are Lipoteichoic acid (LTA, 100 μg/mL), lipopolysaccharide (LPS, 100 ng/ml), and CpG-B ODNs (CpG, 100 nM). Data are presented as mean values±s.e.m. of biological replicates (n=3). Statistical significance was determined using one-way ANOVA.

FIG. 13A-13H shows the immuno-stimulatory activity of GM is TLR4-dependent: FIG. 13A schematic illustration of the signalling pathways evaluated in RAW-Dual™ reporter cells in response to GM stimulation; FIG. 13B WT and Tlr4−/− RAW_Dual™ reporter cells were treated with respective PGNs at indicated concentrations for 24 h for quanti-blue analysis; FIG. 13C WT and Tlr4−/− RAW_Dual™ reporter cells were treated with respective PGNs at indicated concentrations for 24 h for quanti-luciferase analysis LPS (100 ng/ml), MDP (0.4 mM), or Poly(I: C) (0.1 mg/mL) were used as controls. Data are presented as mean values±s.e.m. of biological replicates (n=3). Statistical significance was determined using two-way ANOVA; FIG. 13D Heatmap plot of RNAseq results showing the mean values of differentially expressed genes (p<0.01, and log FC>1) in GM- or MDP-treated WT and Tlr4−/− BMDMs (n=3 biological replicates for each group). WT and Tlr4−/− BMDMs were treated with GM (8 mM) or MDP (0.4 mM) for 24 h; FIG. 13E bar graph showing Il12β mRNA expression in WT and Tlr4−/− BMDMs treated with H₂O, GM, or MDP; FIG. 13F bar graph showing Il1α mRNA expression in WT and Tlr4−/− BMDMs treated with H₂O, GM, or MDP; FIG. 13G bar graph showing TNFα protein concentration in WT and Tlr4−/− BMDMs treated with H₂O, GM, or MDP; and FIG. 13H bar graph showing IL6 protein concentration in WT and Tlr4−/− BMDMs treated with H₂O, GM, or MDP. Data are presented as mean values±s.e.m. of biological replicates (n=4). Statistical significance was determined using one-way ANOVA.

FIG. 14A-14D shows volcano plots of differentially expressed genes and significantly enriched (padj<0.05) KEGG pathways from the RNAseq results of WT BMDMs treated with GM (FIG. 14A) or MDP (FIG. 14C), and Tlr4−/− BMDMs treated with GM (FIG. 14B) or MDP (FIG. 14D). Only the top 20 pathways (by fold enrichment) are shown. Toll-like, NF-κB, NOD-like and TNF signalling pathways are bolded for emphasis. Abbreviations: s.p.=signalling pathway; inf.=infection.

FIG. 15 shows a heatmap plot of RNAseq results showing a selected panel of differentially expressed genes (p<0.01, FDR<0.05, and log FC>1) in WT and Tlr4−/− BMDMs treated with H₂O, GM (8 mM) or MDP (0.4 mM) for 24 h (n=3 biological replicates for each group).

FIG. 16A-16J shows RT-qPCR analysis of GM-induced gene expression in wildtype or Tlr4−/− BMDMs. The GM-induced upregulation of cytokines and receptor genes in WT but not in Tlr4−/− BMDMs validate the RNAseq results in FIG. 13D. WT and Tlr4−/− BMDMs were treated with GM (4 mM) or MDP (0.4 mM) for 24 h. Data are presented as mean values±s.e.m. of biological replicates (n=4). Statistical significance was calculated using one-way ANOVA: FIG. 16A shows Tnfα mRNA expression; FIG. 16B shows Il1β mRNA expression; FIG. 16C shows 116 mRNA expression; FIG. 16D shows Cxcl10 mRNA expression; FIG. 16E shows Nod2 mRNA expression; FIG. 16F shows Tlr4 mRNA expression; FIG. 16G shows Il12α mRNA expression; FIG. 16H shows 1127 mRNA expression; FIG. 16I shows Ifnβ1 mRNA expression; and FIG. 16J shows Ccl5 mRNA expression.

FIG. 17A-17E shows ELISA analysis of GM-stimulated cytokine production in wildtype or Tlr4−/− macrophages: FIG. 17A ELISA analysis of IL-6 secretion in treated WT and Tlr4−/− BMDMs. Cells were challenged with GM (8 mM, 4 mM, 2 mM), M (8 mM), and MDP (0.4 mM) for 24 h. Data are presented as mean values±s.e.m. of biological replicates (n=3-4). Statistical significance was calculated using two-way ANOVA; FIG. 17B ELISA analysis of IL-10 secretion in treated WT BMDMs. Cells were challenged with GM (4 mM), MDP (0.4 mM), and LPS (100 ng/ml) for 24 h. Data are presented as mean values±s.e.m. of biological replicates (n=4). Statistical significance was calculated using one-way ANOVA; FIG. 17C ELISA analysis of IL-6 secretion in treated WT and Tlr4−/− BMDMs;

FIG. 17D ELISA analysis of TNFα secretion in treated WT and Tlr4−/− BMDMs. Cells were challenged with LPS (100 ng/ml) or CpG (100 nM) for 24 h. Data are presented as mean values±s.e.m. of biological replicates (n=4). Statistical significance was calculated using two-way ANOVA; and FIG. 17E ELISA analysis of TNFa secretion in WT and Tlr4−/− RAW_Dual™ cells challenged with GM (2 mM), MG (2 mM), M (2 mM), or MDP (0.4 mM) for 24 h. Data are presented as mean values±s.e.m. of biological replicates (n=3). Statistical significance was determined using two-way ANOVA.

FIG. 18A-18E shows that GM-induced mild activation of NF-κB and MAPK pathways is TLR4-dependent: FIG. 18A Time-course immunoblot analysis of p65 and p38 phosphorylation in WT and Tlr4−/− BMDMs treated with GM (5 mM) or LPS (500 ng/ml), with GAPDH as the housekeeping protein; FIG. 18B-18E Quantifications of pp65 and pp38 against GAPDH in three experimental results as indicated in FIG. 18A; FIG. 18B shows pp65/GAPDH levels in WT BMDMs treated with GM (5 mM) or LPS (500 ng/mL) for 15, 30, 60, or 240 minutes compared with untreated cells; FIG. 18C shows pp65/GAPDH levels in Tlr4⁻/⁻ BMDMs treated with GM (5 mM) or LPS (500 ng/ml) for 15, 30, 60, or 240 minutes compared with untreated cells; FIG. 18D shows pp38/GAPDH levels in WT BMDMs treated with GM (5 mM) or LPS (500 ng/ml) for 15, 30, 60, or 240 minutes compared with untreated cells; and FIG. 18E shows pp38/GAPDH levels in Tlr4/BMDMs treated with GM (5 mM) or LPS (500 ng/ml) for 15, 30, 60, or 240 minutes compared with untreated cells.

FIG. 19A-19E shows that TLR4/MD-2 specifically recognizes GM: FIG. 19A Scheme of the pulldown assay, with GM- or G-immobilized agarose beads; FIG. 19B Coomassie blue-stained SDS-PAGE analysis of mTLR4 proteins bound or unbound to the resin. Excess GM was added to compete off mTLR4 bound to GM-immobilized beads; FIG. 19C Analysis of specific residues in TLR4/MD-2 required for GM-induced NF-κB activity. The various mTLR4/mMD2 variants were transiently expressed in HEK-Blue™ reporter cells and treated with GM (0.4 mM, 0.2 mM) or LPS (100 ng/ml) for 24 h. The activation of NF-κB was measured based on the SEAP reporter signals. For each transfected group, the readouts were normalized to the H2O-treated samples. Data are presented as mean values±s.e.m. of biological replicates (n=3); FIG. 19D chemical structures of GM analogues; and FIG. 19E Structural-activity relationship (SAR) analysis of disaccharide analogues using HEK-Blue™ mTLR4 reporter cells. Cells were treated with respective disaccharides at 0.5 mM or 1 mM. LPS (20 ng/ml and 100 ng/ml) was used as controls. Data are presented as mean values of biological replicates (n=3).

FIG. 20 shows the full coomassie blue-stained gel for in vitro pulldown of mTLR4 by immobilized GM. The scheme and cropped image are shown in FIGS. 19A and 19B.

FIG. 21A-21E shows that surface plasmon resonance (SPR) demonstrating direct binding of GM and mTLR4: FIG. 21A Representative SPR sensorgrams of mTLR4 binding to MDP; FIG. 21B Representative SPR sensorgrams of mTLR4 binding to GM; FIG. 21C Corresponding plots of steady-state binding for MDP and GM; FIG. 21D Table with the estimated respective KD values of steady-state binding for MDP and GM. ND stands for not detected; and FIG. 21E SPR sensorgrams of LPS (500, 250, 120, 62.5, 32.5, 16.2, 0 μg/mL) binding to immobilized TLR4 from three independent experiments. The KD value for LPS binding to TLR4 was estimated. Recombinant mouse TLR4 (26-638)-His6 protein (Cusabio) was immobilized using the standard amine coupling protocol (Biacore, GE Healthcare). Briefly, upon activation of the carboxymethylated dextran surface with 0.05 M NHS/0.2 M EDC, mTLR4 (30 μg/mL, in sodium acetate pH 4.5) was injected into a CM5 sensor chip for immobilization to a level of ˜3000 response unit. The detailed protocol is in the Methods section.

FIG. 22A-22B shows In silico molecular docking of GM to the mTLR4/MD-2 complex; FIG. 22A Molecular docking of GM to the mTLR4-mMD2 complex using AutoDock Vina (DG=−6.1 kcal/mol), with the close-up view of GM-interacting residues shown on the right panel. Images were generated using BIOVIA Discovery Studio software. The mTLR4_K263 residue predicted to interact with the lactoyl group of GM was known for forming hydrogen bonds with polar groups of Lipid41,42. Consistently, mutation of mTLR4_K263A effectively abolished GM-induced NF-κB activation in HEK293T cells (result shown in FIG. 19C); and FIG. 22B The panel of mTLR4/mMD2 variants transiently transfected in HEK293T exhibit comparable expression levels, as demonstrated by Western blots. mTLR4 was FIAG-tagged and mMD2 was c-Myc-tagged.

FIG. 23A-23C shows GM stimulates NF-κB activation in mTLR4 reporter cells but not mTLR2 reporter cells: FIG. 23A SEAP reporter readouts from HEK-Blue™ mTLR4 reporter cells stimulated with GM (2 mM, 1 mM, 0.5 mM), GG (1 mM), GM-A (1 mM), GM-AE (1 mM), LPS (100 ng/ml) or LTA (10 μg/mL) for 24 h; FIG. 23B SEAP reporter readouts from HEK-Blue™ mTLR2 reporter cells stimulated with GM (2 mM, 1 mM, 0.5 mM), GG (1 mM), GM-A (1 mM), GM-AE (1 mM), LPS (100 ng/ml) or LTA (10 μg/mL) for 24 h. Data are presented as mean values of biological replicates (n=4). Statistical significance was determined using one-way ANOVA; and FIG. 23C The disaccharide GM antagonizes LPS-induced NF-kB activation in HEK-mTLR4™ cells. The cells were pre-treated with GM (0.4 mM, 0.2 mM, 0.1 mM, 0 mM) for 24 h, followed by LPS (200 ng/ml, 100 ng/ml, 50 ng/ml) stimulation for 16 h. The SEAP reporter signals were measured as a readout for NFkB activation. For each group, the readouts were normalized to the respective H₂O-treated baseline level. Data are presented as mean values±s.e.m. of biological replicates (n=3). Statistical significance was determined using two-way ANOVA.

FIG. 24A-24M shows the administration of GM reduces colon inflammation in DSS-induced colitis in WT mice but not Tlr4−/− mice: FIG. 24A Timeline of the study, where GM was intraperitoneally administered at 10 mg/kg to mice (WT and Tlr4−/−) that were provided with 3% DSS or normal drinking water (n=6-14). I.p. injections of PBS were given to the negative control group; FIG. 24B Measurements of mice body weight at the endpoint of the timeline; FIG. 24C Measurements of mice colon length at the endpoint of the timeline. Data are presented as mean values±s.e.m. (n=6-14). Statistical significance was determined using two-way ANOVA; FIG. 24D-24G Representative images of colons (FIG. 24D and FIG. 24E) and H&E-stained colon sections (FIG. 24F and FIG. 24G) from each group; FIG. 24H-24J Flow cytometry analysis of immune cell infiltration in colons of each treatment group. FIG. 24H shows the percentage of CD45⁺ cells within pre-gated live cells in wild-type (WT) and Tlr4⁻/⁻ mice treated with PBS, GM, DSS+PBS, or DSS+GM; FIG. 24I shows the percentage of Ly6C⁺ MHCII⁻ monocytes within the CD45⁺ cell population in WT and Tlr4⁻/⁻ mice across the same treatment groups; FIG. 24J shows the percentage of Ly6C⁺ Ly6G⁺ neutrophils within the CD45⁺ cell population in WT and Tlr4⁻/⁻ mice across the same treatment groups. Data are presented as mean values±s.e.m. (n=6-14). Statistical significance was determined using two-way ANOVA; FIGS. 24K-24M RT-qPCR analysis of colonic cytokines demonstrates the significantly reduced inflammation in the GM-supplemented DSS-induced colitis in WT mice but not Tlr4−/− mice; FIG. 24K shows Tnfα mRNA expression normalized to β-actin; FIG. 24L shows Il1b mRNA expression normalized to β-actin; and FIG. 24M shows Ccl2 mRNA expression normalized to β-actin. Data are presented as mean values±s.e.m. (n=6-10). Statistical significance was determined using two-way ANOVA.

FIG. 25A-25E shows GM reduces inflammations in DSS-induced colitis in WT mice but not in Tlr4−/− mice: FIG. 25A Intraperitoneal (i.p.) injections of GM (10 mg/kg) to mice give rise to temporal increases in fecal MurN levels. Mice feces were collected daily before (0 h), 2 h, and 6 h post-GM injection each day for 3 days. Fecal MurN content was quantified following protocol in FIG. 2A-2C. Data are presented as mean values±s.e.m. (n=4); FIG. 25B Histological scores of WT mice in different treatment groups. Scoring was performed based on the severity of colon inflammation, including crypt distortion, epithelial integrity (erosion or ulceration), goblet cell depletion, and cellular infiltration, as shown in the H&E images. The severity was categorized as follows: 0, none; 1, mild; 2, moderate; 3, severe; 4, extremely severe. Data are presented as mean values±s.e.m. (n=5). Statistical significance was calculated using the Kruskal-Wallis test with uncorrected Dunn's test; FIG. 25C Flow cytometry gating strategy for analysis of colon immune cell infiltration; FIG. 25D-25F show RT-qPCR analysis of cytoprotective gene and cytokine expression in colonic tissues from wild-type (WT) and Tlr4⁻/⁻ mice treated with PBS, GM, DSS+PBS, or DSS+GM; FIG. 25D shows Keap1 mRNA expression; FIG. 25E shows Hspβ1 mRNA expression; FIG. 25F shows 1110 mRNA expression. Data are presented as mean values±s.e.m. (n=6-10). Statistical significance was calculated using one-way ANOVA; and FIG. 25G Heatmap of LegendPlex™ analysis of serum inflammatory markers in WT mice in different treatment groups. Data are presented as mean values (n=6-8). Units: pg/mL.

FIG. 26A-26K shows NMR spectrums and characterizations of chemically synthesized PGNs: (FIG. 26A) SI-1; (FIG. 26B) SI-2; (FIG. 26C) SI-3; (FIG. 26D) SI-5; (FIG. 26E) SI-6; (FIG. 26F) NAM-NAG; (FIG. 26G) SI-8; (FIG. 26H) SI-9; (FIG. 26I) SI-10; (FIG. 26J) SI-11, and (FIG. 26K) NAG-NAM.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. Embodiments described below in context of the compounds are analogously valid for the respective compositions, methods, uses, and vice versa. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail. “About”, as used herein in connection with numerical values refers to the referenced numerical value±10% or ±5%.

### TLR4 Agonist Compounds

Gut microbiota-derived peptidoglycan fragments (PGNs) are key signalling molecules that regulate multiple aspects of a host's (or subject's) health. Yet the exact structures of natural PGNs in hosts have not been fully elucidated. The inventors of the present invention obtained a profile of gut microbiota-derived PGNs in hosts, and rather than identifying canonical MDP (i.e. M-AQ) in the host gut; found that the PGN-derived saccharide moieties dominated the gut PGN pool. Unexpectedly, the PGN disaccharide motif, GlcNAc-MurNAc (termed as GM or NAG-NAM hereinafter), was identified and found to elicit mild immuno-stimulatory effects while not activating NOD1/2. Supported by cellular and biochemical evidence as in the working examples, the disaccharide GM was established as a TLR4 agonist, the classic PRR for bacterial lipopolysaccharides (LPS), which highlighted the novel roles of PGN-TLR4 interactions in microbiota-host crosstalk between natural gut microbiota-derived PGNs and host TLR4.

Accordingly, there is provided herein a compound that is capable of activating Toll-like receptor 4 (TLR4) and may be used to stimulate TLR4 and/or a TLR4 response and/or TLR4-mediated signalling cascades. The compound may be termed as a TLR4 agonist that is capable of binding to the TLR4 and eliciting/stimulating a TLR4 response.

Toll-Like Receptors (TLRs), including TLR4, are pattern-recognition receptors (PRRs). TLRs play a well-known role in the initiation of immune responses. At least 10 functional TLRs have been identified in humans. Each TLR detects distinct pathogen associated molecular patterns derived from viruses, bacteria, mycobacteria, fungi, and parasites. Gram-negative bacteria arc typically sensed through the cell wall constituent lipopolysaccharide (LPS) that binds in complex with the LPS-binding Protein (LBP) to a receptor complex of TLR4, CD14 and an associated protein (MD-2). The TLR4-mediated signalling cascades then modulate the gene expression towards the production of a variety of proinflammatory cytokines such as Interleukin (ILV6, Tumour necrosis factor (TNF)-α and IL-1β). In addition, these signalling events enhance the co-stimulatory function of monocytes.

The term “agonist” as used herein, refer to a compound that activates the receptors they bind to and induce its activation and function. As used herein, the term “TLR4 agonist” refers to a compound or molecule that directly or indirectly binds to and activates Toll-like receptor 4 (TLR4), leading to the initiation of downstream signalling pathways that result in immune modulation, including but not limited to the production of pro-inflammatory cytokines, activation of antigen-presenting cells (i.e. macrophages), or enhancement of immune responses. In various embodiments, the compound disclosed herein (i.e. TLR4 agonist) may be referred to as a bioactive PGN motif that acts via NOD1/2-independent pathways. That is, the compound disclosed herein does not bind to or activate NOD1 and/or NOD2.

In various embodiments, the compound disclosed herein (TLR4 agonist) may be a PGN derivative capable of binding to TLR4 and eliciting a TLR4-mediated response, preferably the PGN derivative may be a compound derived from a PGN or analogues, derivatives, all isomer and stereoisomer forms of the compound structure thereof; i.e., regioisomers or the R and S configurations for each asymmetric centre. Therefore, single stereochemical isomers as well as enantiomeric and diastereomeric mixtures of the present compounds are within the scope of the disclosure.

Various compounds of the disclosure possess asymmetric carbon atoms (optical or chiral centres) or double bonds; the enantiomers, racemates, diastereomers, tautomers, geometric isomers, stereoisomeric forms that may be defined, in terms of absolute stereochemistry, as (R)- or (S)-, and individual isomers are encompassed within the scope of the disclosure. The compounds of the disclosure do not include those, which are known in the art to be too unstable to synthesize and/or isolate. The disclosure is meant to include compounds in racemic and optically pure forms. Optically active (R)- and (S)-isomers may be prepared using chiral synthons or chiral reagents, or resolved using conventional techniques. When the compounds described herein contain olefinic bonds or other centres of geometric asymmetry, and unless specified otherwise or prevented by structural constraints, it is intended that the compounds include both E and Z geometric isomers. Additionally, constitutional isomers, including regioisomers, wherein functional groups or substituents may occupy different positions on the molecular framework while maintaining the same molecular formula, are also within the scope of this disclosure.

The compounds disclosed herein that may function as a TLR4 agonist are represented by the formula (I):

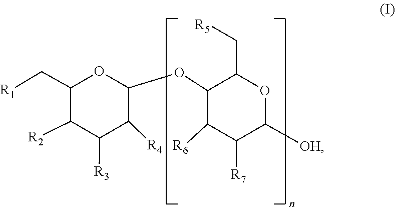

or a pharmaceutically acceptable salt, stereoisomeric form, or regioisomeric form thereof.

In these compounds of formula (I), n may be an integer number selected from 1-4, and R₁, R₂, R₃, R₄, R₅, R₆, R₇ may be independently selected from the group consisting of —H, —OH, —NHC(O)Me, —OC(CH₃)COOH, —NH₂, and —OP(O)(OH)₂.

As used herein, the term “pharmaceutically acceptable salt” refers to those salts which retain the biological effectiveness and properties of the parent compound without being toxic to the subject. Such salts include, but are not restricted to: (1) an acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e. g., an alkali metal ion, such as sodium or potassium, an alkaline earth ion, such as magnesium or calcium, or an aluminum ion; or coordinates with an organic base such as ethanolamine, diethanolamine, triethanolamine, tromethamine, N-methylglucamine, and the like. For more specific, non-limiting examples see, for instance, Berge et al., “Pharmaceutical Salts”, Journal of Pharmaceutical Science, 1977, 66, 1-19).

In various embodiments, R₁, R₂, R₃, R₅, may be —OH, and R₆ may be —OC(CH₃)COOH.

In various embodiments, one or both of R₄ and R₇ may be —NHC(O)Me.

In various embodiments, the compound may be a disaccharide compound, and thus in formula (I) n may be 1. A “disaccharide” is defined herein refers to a compound comprising two monosaccharide units covalently linked by a glycosidic bond, for example, the monosaccharides may be N-acetylglucosamine (GlcNAc) and N-acetylmuramic acid (MurNAc), or analogs. The disaccharide compound may exist in various regioisomeric forms, wherein the relative positions of the sugar units and/or their glycosidic linkages may differ while maintaining the core disaccharide structure. Additionally, the compound may include tautomers, anomers (a or B configurations), and stereochemical variants without departing from the scope of the disclosure.

In various embodiments, the compounds disclosed herein may be a compound of formula (II), having the formula:

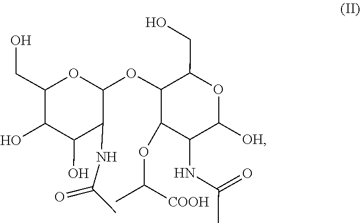 [SMILES: CC(=O)NC1C(OC2C(CO)OC(O)C(NC(C)=O)C2OC(C)C(=O)O)OC(CO)C(O)C1O]

or a pharmaceutically acceptable salt, stereoisomeric form, or regioisomeric form thereof.

In various embodiments, the compounds disclosed herein may be a compound of formula (III), having the formula:

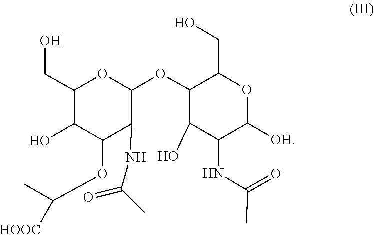 [SMILES: CC(=O)NC1C(O)OC(CO)C(OC2OC(CO)C(O)C(OC(C)C(=O)O)C2NC(C)=O)C1O]

or a pharmaceutically acceptable salt, stereoisomeric form, or regioisomeric form thereof.

In various embodiments, the compounds disclosed herein may be GlcNAc-MurNAc (GM) having the formula:

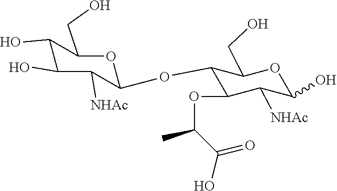 [SMILES: CC(=O)NC1C(OC2C(CO)OC(O)C(NC(C)=O)C2O[C@H](C)C(=O)O)OC(CO)C(O)C1O]

or a pharmaceutically acceptable salt, analogue, stereoisomeric form, or regioisomeric form thereof.

The TLR4 agonist termed GM (GlcNAc-MurNAc) refers to a disaccharide consisting of N-acetylglucosamine (GlcNAc) linked to N-acetylmuramic acid (MurNAc), with the GlcNAc unit positioned at the reducing end and MurNAc at the non-reducing end. The C1 of GlcNAc (N-acetylglucosamine) is linked to the C4 of MurNAc (N-acetylmuramic acid) through a β-(1→4) glycosidic bond.

In various embodiments, the compounds disclosed herein may be a regioisomer form of GlcNAc-MurNAc (GM) having the formula:

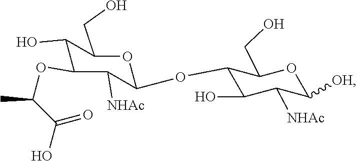 [SMILES: CC(=O)NC1C(O)OC(CO)C(OC2OC(CO)C(O)C(O[C@H](C)C(=O)O)C2NC(C)=O)C1O]

or a pharmaceutically acceptable salt, or analogue thereof. MG (MurNAc-GlcNAc) is a disaccharide and the regioisomer of GM, where the MurNAc unit is at the reducing end, and GlcNAc is at the non-reducing end. Compared to GM, the MG disaccharide includes the order of monosaccharides as being MurNAc followed by GlcNAc, whereby the C4 of GlcNAc (N-acetylglucosamine) is linked to the C1 of MurNAc (N-acetylmuramic acid) through a β-(1→4) glycosidic bond.

As used herein, the term “Ac” in the context of the compound and substituents refers to an acetyl group denoted by the chemical formula —COCH₃.

As used herein, the term “binds” refers to the interaction between a compound disclosed herein and the target TLR4. The terms “interact” and “couple” may be used interchangeably with “bind’ in the context of the invention. In various embodiments, the nature of the binding interaction may be “direct binding”, meaning that the compound is interacting with the TLR4 without any intermediaries or additional molecules to facilitate the binding, and is a one-on-one interaction. This is in contrast to indirect binding, where other molecules or co-factors might be needed for a compound to effectively bind to the TLR4.

In various embodiments, the compound disclosed herein demonstrates selective interaction with TLR4 (i.e. selectively binds), resulting in the activation or modulation of TLR4-mediated signalling pathways, without substantial binding or activation of other Toll-like receptors (TLRs), including but not limited to TLR1, TLR2, TLR3, TLR5, TLR7, TLR8, and TLR9. In various embodiments, the compound disclosed herein selectively binds to TLR4, and does not bind to or activate other TLRs. Accordingly, the compound disclosed herein exhibits selective functional activation, in that it preferentially activates TLR4-mediated cellular responses, such as cytokine production or NF-κB signalling, as demonstrated by cellular assays (e.g., reporter assays, ELISA, or flow cytometry), while showing negligible or no activation of other TLRs.

In various embodiments, the compound disclosed herein specifically binds to TLR4. In this regard, the compound may recognize and bind to the TLR4 with a binding affinity or specificity that is higher than that for other compounds that may be present. As used herein, the term “affinity” refers to the strength of binding of a compound to a target molecule. In this regard, the term “specifically binds” may refer to the selective binding affinity the compound exhibits (e.g., dissociation constant, Kd) for TLR4 that is at least 2, 3, 4, 5, 6, 7, 8, 9 or 10-fold greater than its affinity for other TLRs under identical conditions, as determined by standard biochemical or biophysical assays (e.g., surface plasmon resonance, isothermal titration calorimetry, or competition binding assays).

In various embodiments, the compound disclosed herein may have a binding affinity to TLR4, and represents a TLR4-selective binding ligand. The binding affinity may be expressed in terms of a dissociation constant (K_{d}), whereby the lower the K_{d}, the higher the binding affinity of the binding ligand to the target. In various embodiments, the dissociation constant (K_{d}) of a complex in which the compound and target TLR4 are bound may be 500 μM or less, 450 μM or less, 400 μM or less, 390 μM or less. In various embodiments, the compound disclosed herein binds to TLR4 with a K_{d} of about 300 μM.

In various embodiments, the compound binds to TLR4 with at least about a 10⁶-fold greater affinity, preferably at least about a 10⁷-fold greater affinity, more preferably at least about a 10⁸-fold greater affinity, and most preferably at least about a 10⁹-fold greater affinity than it binds to other TLRs. Typically, specific binding refers to affinities in the range of about 10⁶-fold to about 10⁹-fold greater than non-specific binding. In various embodiments, specific binding may be characterized by affinities greater than 10⁹-fold over non-specific binding.

In various embodiments, the compound disclosed herein binds to TLR4 with greater affinity or greater specificity or both than to other TLRs (e.g. TLR2). In this context, the term “greater affinity” indicates that the compound binds more tightly to TLR4 that to other TLRs, i.e., with a lower dissociation constant. In various embodiments, the greater affinity is at least 2, 3, 4, 5, 8, 10, 50, 100-fold greater affinity. Also in the context of compounds binding to TLR4, the term “greater specificity” indicates that the compound binds to a specified target to a greater extent than to another TLRs that may be present under relevant binding conditions.

In various embodiments, the compound disclosed herein is not a naturally occurring product. In various embodiments, the compound disclosed herein is free of any attached peptide chains, or crosslinking groups, including but not limited to stem peptides (e.g., L-alanine, D-glutamic acid, meso-diaminopimelic acid, or L-lysine), and crosslinked peptide bridges or peptide derivatives typical of bacterial cell wall structures. In this regard, GlcNAc-MurNAc (GM) is naturally attached to a peptide in forming muropeptides. The monosaccharide of the compound are free from, and do not contain structural modifications characteristic of muropeptide disaccharides. In this regard, the compound lacks any attached peptide chains, amino acid substitutions, or modifications typically associated with peptidoglycan biosynthesis, including but not limited to stem peptides, crosslinked peptide bridges or any derivatives thereof, and any chemical or enzymatic peptide modifications that introduce peptide-based functional groups. In various embodiments, the TLR4 agonist is a disaccharide and does not encompass a disaccharide-muropeptide such as GM-A or GM-AE. In various embodiments, the TLR4 agonist consists of the compound disclosed herein. The compound disclosed herein is not linked to any peptide and is presented as a synthetic or isolated, purified compound that is devoid of the naturally occurring stem peptide typically found in bacterial peptidoglycan. This structural distinction confers markedly different properties, including selective TLR4 agonist activity, absence of NOD1/2 activation, and enhanced pharmaceutical utility as a standalone compound.

In various embodiments, the compound disclosed herein may be naturally derived and isolated or synthetically produced through any suitable means and synthesis methods known to the skilled person. Accordingly, in various embodiments, the compound is a naturally-derived and isolated compound or the compound is a synthetic compound.

In various embodiments, the compound disclosed herein may be naturally derived from a host cell or cell culture. The host may be any suitable bacterial cell (e.g., Gram-positive or Gram-negative bacteria known to produce peptidoglycan, such as Escherichia coli or Staphylococcus aureus). The bacterial cells may be cultured and grown in a suitable culture medium until they reach the desired growth phase. The cultured bacterial cells may then be lysed (e.g. via a lysis buffer) to release the cell content, including peptidoglycan fragments. The compounds disclosed herein may then be isolated from the peptidoglycan fragments, using any suitable means to remove cytoplasmic contents and any non-peptidoglycan-associated compounds. The compounds disclosed herein may then be purified to remove any peptide residues or structural modifications typically present in the compounds natural form, as well as remove components from a composition or host cell or culture, the presence of which is not desired. Purification is a relative term and does not require that all traces of the undesirable component be removed from the composition. Thus, the term “purified” does not require absolute purity; rather, it is intended as a relative term. Confirmation and validation that the compounds disclosed herein have been obtained, may be performed using standard analytical techniques such as Mass spectrometry (MS), Nuclear magnetic resonance (NMR), and/or High-performance liquid chromatography (HPLC). The absence of endotoxin contaminations in isolated/synthetic compound disclosed herein may be validated by Limulus Amebocyte Lysate (LAL) assay and by titration with polymyxin B.

In various embodiments, the compound disclosed herein may be synthetically produced and prepared using the reaction routes and synthesis schemes as described below in the materials and methods section of the working examples, employing the techniques available in the art using starting materials that are readily available. In various embodiments, the compound disclosed herein may be synthetically produced via chemical or enzymatic synthesis without incorporation of peptide residues or modifications characteristic of bacterial peptidoglycan biosynthesis. Confirmation and validation that the compounds disclosed herein have been obtained, may be performed using standard analytical techniques such as Mass spectrometry (MS), Nuclear magnetic resonance (NMR), and/or High-performance liquid chromatography (HPLC). The purified compound may be lyophilized to obtain a stable, dry product. In various embodiments, the compound disclosed herein is a synthetic compound, and is not naturally derived.

The invention also relates to compositions comprising the compounds disclosed herein. Embodiments described in the context of the compound are analogously valid for the respective compositions comprising said compound, and vice versa. As contemplated herein, the terms “composition” and “formulation” may be used interchangeably.

In various embodiments, the composition is to be administered orally for delivery to the gastrointestinal tract of the subject, particularly the colon. To facilitate targeted delivery, the composition may incorporate specialized carriers or delivery systems. These may include excipients and carriers broadly categorized as biopolymeric drug delivery systems, lipid-based nanocarriers, microparticles, nanoparticles, encapsulation systems, and controlled or targeted release vehicles, designed and formulated to protect, stabilize, and/or facilitate the targeted release of the compound within specific regions of the GI tract, such as the stomach, small intestine, or colon. For example, the composition may incorporate pectin/zein beads, which are classified as biopolymeric microparticles composed of natural polysaccharides (e.g., pectin) and plant-derived proteins (e.g., zein), designed for colon-targeted or sustained release of the compound disclosed herein via pH or microbial degradation mechanisms. Alternatively or additionally, the composition may include liposome nanoparticles, which are lipid-based nanocarriers formed from phospholipid bilayers that encapsulate hydrophilic or lipophilic active compounds, offering protection from enzymatic degradation and facilitating controlled or mucosal delivery within the GI tract. Additional formulation components may include polysaccharides (e.g., alginate, chitosan, guar gum), biodegradable polymers (e.g., polylactic acid, polycaprolactone), surfactants, stabilizers, emulsifiers, pH-sensitive coatings (e.g., Eudragit®), and other excipients that enhance bioavailability, stability, and mucosal adhesion, or modulate release kinetics in accordance with the physiological conditions of the GI tract.

In various embodiments, the composition may be a pharmaceutical composition for use in administering to a subject.

The pharmaceutical composition may comprise the compound disclosed herein and a pharmaceutically acceptable carrier and/or excipient. The compounds of the invention are thus contemplated for use as a pharmaceutical. The purpose of a pharmaceutical composition is to facilitate administration of a compound to a subject. Pharmaceutical compositions comprising a compound disclosed herein in free form or in a pharmaceutically acceptable salt form in association with at least one pharmaceutically acceptable carrier or diluent can be manufactured in a conventional manner by mixing, granulating, or coating methods.

As used herein, a “pharmaceutically acceptable carrier” refers to a carrier or diluent that does not cause significant irritation to an organism and does not abrogate the biological activity and properties of the. administered compound. A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

In various embodiments, the pharmaceutical compositions are formulated to be suitable for oral administration and may be presented as discrete dosage units (such as pills, tablets, pellets, capsules, or softgel capsules), as a powder or granule, or as a liquid form, for example solution, suspension, syrup, or elixir. Solutions/suspensions may be formulated for intravenous administration.

In various embodiments, the composition may be a nutraceutical composition for use in administering to a subject.

As used herein, the term “nutraceutical composition” refers to a formulation containing the compound disclosed herein to provide medicinal or health benefits beyond basic nutrition but do not meet the regulatory definition of pharmaceuticals. The nutraceutical composition of the present invention includes the compound disclosed herein, which may be combined with other bioactive compounds such as polyphenols, flavonoids, adaptogens, peptides, or lipid-based carriers to enhance its physiological effects. In this regard, the nutraceutical composition may be used for disease prevention, health maintenance, or physiological enhancement, and unlike pharmaceuticals, nutraceutical compositions are not intended to diagnose, treat, or cure diseases or conditions. Such nutraceutical compositions include, but are not limited to: a food additive, a food supplement, a dietary supplement, genetically engineered foods (such as for example vegetables, herbal products, and processed foods such as cereals, soups, and beverages), stimulant functional food, medical food, parenteral nutrition, and pharmafood (also sometimes designated “phood”). Dietary supplements may be delivered in the form of soft gel capsules, tablets, syrups, and other known dietary supplement delivery systems. The nutraceutical composition may be in any of the many dosage delivery forms commonly used in the art. A medical food as used herein is specially formulated and intended for the dietary management of a disease/disorder that has distinctive nutritional needs that cannot be met by normal diet alone. While the effects of such a nutraceutical composition may contribute to overall well-being and digestive function, any potential role in managing gastrointestinal conditions is supportive rather than therapeutic.

In various embodiments, the nutraceutical composition may comprise pectin/zein beads or liposome nanoparticles and is formulated to be orally administered and the compound delivered to the GI tract, and particularly the colon, of the subject.

In various embodiments, the composition may be a supplement composition for use in administering to a subject.

As used herein, the term “supplement composition” or “dietary supplement composition” refers to a composition formulated to complement the diet of a subject by providing the compound disclosed herein with optionally one or more bioactive ingredients, including but not limited to vitamins, minerals, amino acids, herbal extracts, probiotics, prebiotics, peptides, or other functional compounds, designed to support general health, wellness, or specific physiological functions, as opposed to treating, curing or preventing any disease/condition (i.e. non-therapeutic utility). The supplement composition may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the supplement may contain an organic or inorganic carrier material suitable for oral or parenteral administration. In this regard, a “supplement composition” may be used to complement the nutrition of a subject. The supplement composition may be prepared in any suitable manner and formulated to enhance bioavailability, metabolic support, or targeted physiological benefits of the compound disclosed herein. The supplement composition may be in the form of a capsule, tablet, powder, liquid, or any other suitable oral, topical, or injectable delivery format.

In various embodiments, the supplemental composition may comprise pectin/zein beads or liposome nanoparticles and formulated to be orally administered and the compound delivered to the GI tract, and particularly the colon, of the subject.

In various embodiments, there is provided a nutritional composition comprising the compound disclosed herein.

As used herein, the term “nutritional composition” refers to a composition which nourishes a subject, and may refer to a food-based composition or dietary product that provides essential or functional nutrients intended to support overall health, energy balance, or specific physiological needs. The nutritional composition may be prepared in any suitable manner and formulated to improve immune function, and gut microbiome interactions. The nutritional composition is not particularly limited as long as it is suitable for administration (e.g. oral or intravenous administration). Examples of suitable nutritional compositions include foodstuffs, drinks, drug bases, and animal feeds. The nutritional composition according to the invention may be an enteral nutritional composition. An “enteral nutritional composition” is a foodstuff that involves the gastrointestinal tract for its administration. The nutritional composition may be suitable for infants. For example the nutritional composition may be an infant formula, a baby food, an infant cereal composition, or a fortifier. Suitably, the nutritional composition may be an infant formula or a fortifier.

In various embodiments, the nutritional composition may comprise pectin/zein beads or liposome nanoparticles and formulated to be orally administered and the compound delivered to the GI tract, and particularly the colon, of the subject.

### Methods of Use

The invention also relates to methods of using the compounds and compositions disclosed herein. Embodiments described in the context of the compound and compositions are analogously valid for the respective methods and uses described below, and vice versa.

As a TLR4 agonist, the compounds disclosed herein activate Toll-like receptor 4 (TLR4), leading to the initiation of downstream signalling pathways that result in immune modulation, including but not limited to the possible production of pro-inflammatory cytokines, activation of antigen-presenting cells, or enhancement of adaptive immune responses. Thus, the compounds disclosed herein may be termed as an immunostimulatory compound used in methods for stimulating immune cells and/or eliciting an immune response either in vitro, ex vivo or in vivo.

The compounds disclosed herein may be defined as being immunogenic. Being “immunogenic” means that the compound elicits an immune response when administered to a subject (e.g., a mammalian subject such as a human). As used herein, an “immune response” refers to a response by a cell of the immune system, such as an antigen-presenting cell, dendritic cell, monocyte, macrophage, NKT cell. NK cell, basophil, eosinophil, or neutrophil, B cell, T cell (CD4 or CD8), regulatory T cell, antigen-presenting cell, dendritic cell, monocyte, macrophage. NKT cell, NK cell, basophil, eosinophil, or neutrophil, to a stimulus (e.g., to the compound disclosed herein).

In various embodiments, the methods for stimulating immune cells and/or eliciting an immune response may be either in vivo, ex vivo or in vitro methods.

Accordingly, in various embodiments there is provided a method of enhancing or increasing TLR4 activity, comprising contacting an effective amount of a compound or composition disclosed herein, with the TLR4. As used herein, the terms ‘increase’ or ‘enhance’ in relation to TLR4 activity refer to a rise in TLR4 activation or signalling response upon contact with the disclosed compounds or composition, relative to a reference activity level of TLR4 or its signalling response. The compound or composition is contacted with TLR4 of a cell (e.g. macrophages, dendritic cells, and epithelial cells) to activate the TLR4 signaling cascade and stimulate immune cells and/or elicit an immune response.

In various embodiments, the compound disclosed herein may be defined as a mild-TLR4 agonist in that it elicits mild immunostimulatory effects. A mild-stimulatory effect of the TLR4 agonist may refer to a moderate activation of the Toll-like receptor 4 (TLR4) signalling pathway, leading to a balanced immune response. Unlike a strong effect, which triggers robust production of pro-inflammatory cytokines (e.g., TNF-α, IL-6) and may induce excessive inflammation or systemic immune activation, a mild effect results in a controlled cytokine release that enhances immune function without excessive immune-related toxicity. Compared to a weak effect, where minimal activation may fail to elicit a meaningful immune response, a mild stimulation ensures sufficient immune signalling for pathogen recognition or therapeutic modulation while minimizing adverse effects. This level of stimulation is particularly relevant in vaccine adjuvant development or immunotherapy, where a fine-tuned TLR4 response can enhance immunity without triggering excessive inflammation or immune exhaustion.

In various embodiments, the immune response elicited by the compound disclosed herein is an innate and/or adaptive immune response. An “innate immune response” refers to a non-specific, immediate immune defence mechanism that serves as the first line of protection against pathogens. This response is mediated by innate immune cells, including macrophages, dendritic cells, neutrophils, and natural killer (NK) cells, which recognize conserved pathogen-associated molecular patterns (PAMPs) through pattern recognition receptors (PRRs) such as Toll-like receptors (TLRs). The innate immune response involves the activation of inflammatory pathways, cytokine and chemokine secretion, and phagocytosis, leading to the rapid elimination of invading pathogens and the initiation of subsequent adaptive immune responses. An “adaptive immune response” refers to a highly specific and long-lasting immune defines mechanism that develops in response to antigen exposure. Unlike the innate immune response, the adaptive immune response is characterized by antigen specificity, immunological memory, and the ability to mount a more robust response upon re-exposure to the same antigen. It is mediated by lymphocytes, including B cells, which produce antigen-specific antibodies, and T cells, which contribute to cellular immunity through direct cytotoxic activity or helper functions.

In various embodiments, the cell(s) may be in vitro and the compound or composition is administered such that the compound contacts with the cell, and TLR4, under suitable conditions, and the cell may be a cell from (or derived from, in case of cell cultures) a multicellular eukaryote such as a human cell line or another mammalian cell line. The mammalian cell lines can include, but are not limited to a human, simian, murine, mice, rat, monkey, rabbit, rodent, hamster, goat, bovine, sheep, or pig cell lines. It will be appreciated that the cell or cell lines is one which expresses the TLR4 to be modulated. In various embodiments, the cell is from a cell line including, but not limited to a cancer cell lines, immune cell lines, epithelial cell lines, fibroblast and mesenchymal cell lines or enteroendocrine and goblet cell lines. In various embodiments, the cell may be in vitro and is a cell from a biological sample that has been obtained from, removed, or isolated from a subject using suitable and readily known procedures to process and prepare said samples, as will be appreciated by those skilled in the art. The biological sample may be a sample of tissue or cells from the subject, for example, tissue or cells may be obtained and isolated from the gastrointestinal tract of the subject.

The “contacting” step in the in vitro methods generally refers to any suitable means for delivering, exposing or bringing the compound and TLR4s together in a controlled experimental setting under suitable conditions. As will be appreciated by the skilled person, the “contacting” step can vary depending on the nature of the cells used and compound or composition involved, whereby the contacting may involve physically mixing the cells with the compound in a test tube, petri dish or other container, or it could involve applying the compound to the cells through techniques such as pipetting, incubation, or surface immobilization, or it could involve introducing the compound into the cells, such as through transfection. The purpose of the “contacting” step is to allow the compound to interact with the TLR4s and exert its modulatory effect. In various embodiments, contacting occurs in a solution in which the cells and compounds are mixed in a common solution and are allowed to freely associate, or the contacting can occur at or otherwise within a suitable cell environment.

It will be appreciated that such in vitro methods may be used to investigate TLR4 activation (intracellular or extracellular) in cell culture for the purpose of immune system and inflammation studies, drug screening and development by screening anti-inflammatory or immunomodulatory drugs to assess their effects on TLR4-mediated signalling pathways, and potential therapeutic use, and/or adjuvant testing of the compounds to assess immune-stimulating adjuvant properties for potential vaccine formulations.

In various embodiments, the cell may be in vivo, i.e. the cell may be within a subject, wherein the method comprises administering to the subject an effective amount of the compound or composition disclosed herein for enhancing or increasing TLR4 activity. Accordingly, the compound or composition disclosed herein may be used in methods for stimulating immune cells and/or eliciting an immune response in a subject. In various embodiments, the compounds disclosed herein can be administered by any conventional route, particularly enterally, orally, or parenterally, in the form of injectable solutions or suspensions, or in a nasal or suppository form. Depending on the route of administration, the compound or composition may be coated with a material to protect the compound from the action of enzymes, acids and other natural conditions which may inactivate the therapeutic activity of the compound. In various embodiments, the compound or composition disclosed herein can be administered enterally, orally or parenterally. In various embodiments, the compound or composition disclosed herein is administered orally, and formulated accordingly.

In various embodiments, the compound or composition disclosed herein may be used as an adjuvant. Adjuvants can enhance, prolong, and modulate immune responses to maximize protective immunity. The use of the compound as an adjuvant may enable effective immunization in vulnerable populations (e.g., neonates, elderly, or immunocompromised individuals). In various embodiments, the compound or composition disclosed herein may be for use as an adjuvant in a vaccine.

An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system see, e.g., H. S. Warren et al, Anna. Rev. Immunol., 4:369 (1986), incorporated herein by reference. The ability of an adjuvant to induce and increase a specific type of immune response and the identification of that ability is thus a key factor in the selection of particular adjuvants for vaccine use against a particular pathogen.

The invention also relates to the compound or composition disclosed herein for use as a medicament in therapy.

Compounds disclosed herein may be administered to a subject in therapeutically effective amounts either alone or in combination with one or more additional therapeutic agents (pharmaceutical combinations). In particular, the compounds may be used in the context of cancer immunotherapy, where activation of innate immune pathways via TLR4 signaling may enhance antitumor immune responses. When administered in conjunction with other therapies, such as immune checkpoint inhibitors (e.g., anti-PD-1, anti-CTLA-4 antibodies), chemotherapeutic agents, cytokines, cancer vaccines, or other immunomodulatory compounds, the co-administration is intended to improve the overall therapeutic outcome. Where the compounds disclosed herein are administered in conjunction with other therapies, dosages of the co-administered compounds will of course vary depending on the type of co-drug employed, on the specific drug employed, on the condition being treated and so forth.

In various embodiments, the compound or composition disclosed herein may be used in methods of treating or preventing a condition or disease associated with aberrant TLR4 activity, comprising administering a therapeutically or prophylactically effective amount of a compound disclosed herein, to a subject in need thereof. This aspect also covers uses of the compound or composition disclosed herein in the manufacture of a medicament for the treatment or prevention of a condition or disease associated with aberrant TLR4 activity in a subject in need thereof, wherein said prevention or treatment may comprise administering a therapeutically or prophylactically effective amount of the compound.

An “effective amount”, as used herein, relates to an amount that is sufficient to provide a desired effect, including preventing, reducing the risk of being afflicted by, alleviating and abating a disease and/or its attendant symptoms. This applies to terms used herein, such as “therapeutically effective amount” (alleviating and abating a disease and/or its attendant symptoms) and “prophylactically effective amount” (preventing, reducing the risk of being afflicted by a disease and/or its attendant symptoms). In general, compounds disclosed herein will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors.

As used herein, the term “prevention”, as well as related terms such as “prevent” or “preventing,” is meant to refer to provide a subject not yet affected by the condition with a benefit that serves to avoid, delay, forestall, minimize, or reduce the recurrence/onset of the condition to be prevented and/or its attendant symptoms. Such preventative benefits include, for example, delaying development and/or recurrence of the condition, or reducing the duration, severity, or intensity of one or more unwanted features associated with the condition if it eventually develops.

As used herein, the terms “treating” and “treatment” refer to reduction in severity and/or frequency of symptoms, elimination of symptoms and/or underlying cause, prevention of the occurrence of symptoms and/or their underlying cause, and improvement or remediation of damage. As used herein, the term “preventing” refers to the prophylactic or preventative measures that prevent and/or slow the development of a targeted pathologic condition or disorder. Thus, those in need of treatment include those already with the disorder; those prone to have the disorder; and those in whom the disorder is to be prevented and those in whom reoccurrence of the disorder needs to be prevented. In various embodiments, the terms “treating”, “ameliorating”, “delaying” or “preventing”, as used herein refer to achieving one or more of the following in the subject: (a) reducing the severity of a given condition; (b) limiting or preventing the development of a condition; (c) removing a given condition; (d) limiting or preventing the recurrence of a given condition; (e) alleviation of the condition and/or its symptoms; and (f) delay the onset of a condition. Any one or more of these effects may be achieved in a subject who previously had or currently has or is suspected to develop a glucose metabolism disease or condition. In particular, the therapeutic terms “treating, ameliorating or preventing”, may refer to reducing the likelihood of a particular condition or disease state from occurring in a subject not presently experiencing or afflicted with the condition or disease state. The terms do not necessarily indicate complete or absolute prevention or treatment.

As used herein, the term “condition or disease associated with aberrant TLR4 activity” may include but are not limited to chronic inflammatory disorders, autoimmune diseases, infections, metabolic disorders, and cancer. For infectious diseases, TLR4 agonists can enhance innate immune responses, improving host defence against bacterial, viral, and parasitic infections. For cancer immunotherapy, TLR4 agonists can stimulate anti-tumour immunity by activating dendritic cells (DCs) and macrophages, leading to stronger T-cell responses. For immunodeficiency and chronic Infections, TLR4 agonists can help revitalize immune function in subjects with immune suppression. In various embodiments, the cancer may be gastrointestinal cancer, preferably colon cancer. Colon cancer, may be referred to as colorectal adenocarcinoma when encompassing both colon and rectal cancers, and is a subtype of gastrointestinal cancer and is generally characterized by uncontrolled cellular proliferation, local tissue invasion. Cancers related to colon cancer include rectal cancer, which arises in the distal part of the large intestine, and collectively with colon cancer is termed colorectal cancer (CRC), thus the cancer may be CRC. Additional related malignancies include cancers of the small intestine, anal canal, appendix, and peritoneum, which, while less common, may share histopathological features, genetic mutations, or therapeutic strategies with colon cancer.

In consideration that Toll-like receptor 4 (TLR4) plays a crucial role in the innate immune system, particularly in modulating immune responses within the gastrointestinal (GI) tract, it will be appreciated that TLR4 activation by the compound or composition disclosed herein may thus be used in methods for maintaining or improving gut homeostasis. Accordingly, the compound or composition disclosed herein may be used in methods of promoting or maintaining gastrointestinal health in a subject, and/or modulating gut microbiota in a subject comprising administering the compound or composition to the subject. This aspect also covers uses of the compound or composition disclosed herein in the manufacture of a medicament for maintaining or improving gut homeostasis in a subject in need thereof, wherein said use comprises administering a therapeutically or prophylactically effective amount of the compound.

It is also contemplated that the compound or composition disclosed herein may be used as a postbiotic for administering to a subject in order to promote, improve or maintain gastrointestinal health. Accordingly, in various embodiments, the compound or composition may be formulated as a postbiotic. Postbiotics offer potential alternatives or adjunctive therapies to the use of live microorganisms and their metabolites. The term “postbiotic” as used herein refers to a bioactive compound that is derived from microbial metabolism or synthetically produced to mimic the functional properties of microbial metabolites, thereby conferring health benefits to the subject or host without requiring the presence of live microorganisms. Specifically, a postbiotic is a non-viable, bioactive compound that can be obtained through microbial fermentation or synthesized using chemical or enzymatic methods while retaining its physiological activity (i.e. TLR4 agonist activity).

In various embodiments, the compound may be formulated as a postbiotic and incorporated into an edible material using a standard technique well known to one of ordinary skill in the art. For instance, the compound may be directly added to the edible material, or may be utilized for preparing an intermediate composition (e.g., a food additive or a premix) suitable to be subsequently added to the edible material. The food product may be in the form of fermented foods, processed foods, health foods, or dietary supplements. In addition, the postbiotic may be prepared in the form of a pharmaceutical composition.

As used herein, the promotion, improvement or maintenance of gastrointestinal health refers to a subject, who upon administration of the compound (e.g. formulated as a postbiotic), exhibits a healthy gut/intestinal microbiota, which is beneficial for human or animal health and suitable for a maintenance and/or an improvement of the digestion of said subject.

Gut microbiota dysbiosis can inflict dysregulated immune responses in subjects, leading to chronic inflammatory diseases such as inflammatory bowel diseases (IBD). It follows that the TLR4-mediated immune responses elicited by the compounds disclosed herein acting as TLR4 agonists may also hold significant potential in the treatment of gut-associated inflammatory diseases whereby collected data suggests that the compound or composition may maintain gut homeostasis via TLR4-dependent mechanisms and protect against colonic injury and alleviate colonic inflammation. Specifically, the administration of the compound or composition disclosed herein may be used to treat or prevent colitis in subjects via TLR4-dependent mechanism(s).

Accordingly, in various embodiments, the compound or composition disclosed herein may be used in methods of treating, preventing, or ameliorating a gastrointestinal disease, comprising administering the compound or composition disclosed herein to a subject in need thereof. This aspect also covers uses of the compound or composition disclosed herein in the manufacture of a medicament for the treating, preventing, or ameliorating a gastrointestinal disease in a subject.

The term “subject”, as used herein in the context of therapeutic methods, refers to a warm-blooded animal, preferably a mammal, more preferably a human. Said subject may be awaiting or receiving medical care or is or will become the subject of a medical procedure or is being monitored for the development of a gastrointestinal condition or any condition or disease associated with aberrant TLR4 activity. Subjects include those already being afflicted by gastrointestinal condition or any condition or disease associated with aberrant TLR4 activity as well as subjects susceptible to gastrointestinal condition or any condition or disease associated with aberrant TLR4 activity or for whom gastrointestinal condition or any condition or disease associated with aberrant TLR4 activity should be prevented or delayed. In the context of therapeutic methods described herein, subjects may be termed as “non-healthy subjects”.

As used herein, a “gastrointestinal disease” (also known as “GI disease” or “GI illness”) may refer to diseases involving the gastrointestinal tract, which includes the oesophagus, stomach, small intestine, large intestine and rectum. In various embodiments, the gastrointestinal disease is a gastric disease or an intestinal disease. In various embodiments, the gastrointestinal disease is a gastric disease. A “gastric disease’ may refer to diseases affecting the stomach. In some embodiments, the gastrointestinal disease is an intestinal disease. An “intestinal disease” may refer to diseases affecting the small intestine (including the duodenum, jejunum, and ileum) or large intestine (including the cecum, colon, and rectum). In various embodiments, the gastrointestinal disease is selected from: antibiotic-associated diarrhea, Helicobacter pylori infection, an inflammatory bowel disease (IBD), irritable bowel syndrome (IBS), lactose intolerance, infectious diarrhea, colorectal cancer, chemotherapy-induced diarrhea, and necrotizing enterocolitis.

In various embodiments, the gastrointestinal disease is selected from: antibiotic-associated diarrhea, Helicobacter pylori infection, an inflammatory bowel disease (IBD), and irritable bowel syndrome (IBS).

In various embodiments, the gastrointestinal disease is an inflammatory bowel disease (IBD). Inflammatory bowel disease (IBD) is a group of inflammatory conditions of the colon and small intestine. Exemplary IBDs include Crohn's disease (CD), ulcerative colitis (UC), and pouchitis. It has been suggested that dysbiosis (that is, abnormal microbiota composition) and decreased complexity of the gut microbial ecosystem are common features in patients with IBD (see Manichanh, C., et al., 2012. Nature reviews Gastroenterology & hepatology, 9 (10), pp. 599-608).

In various embodiments, the Inflammatory bowel disease (IBD) is colitis.

As will be appreciated, the present invention may also provide a method for using probiotic microorganisms to produce and release the compound disclosed herein (i.e. TLR4 agonist) within the gastrointestinal (GI) tract of a subject, thereby modulating immune responses and promoting gut health. The method may comprise the selection, modification, and administration of probiotic strains capable of expressing and secreting the compound disclosed herein, which can stimulate the immune system to enhance mucosal immunity, promote gut homeostasis, and provide therapeutic benefits in conditions such as dysbiosis, and gastrointestinal diseases.

Accordingly, it is also contemplated that there is provided a probiotic microorganism (strain) that has been engineered to produce and secrete the compound disclosed herein in the GI tract of a subject. Upon administration to the subject, the probiotic microorganism may colonize the gastrointestinal tract and secrete the compound.

In various embodiments, the method comprises selecting probiotic microorganism strains that naturally or through engineering have the capacity to produce the compound disclosed herein. Suitable probiotic strains (probiotic microorganism) include, but are not limited to, Lactobacillus, Bifidobacterium, Bacillus, Staphylococcus and Enterococcus species. These probiotics may be sourced from natural isolates or modified using synthetic biology techniques to enhance their capabilities of producing the compound disclosed herein. In various embodiments, suitable probiotic strains include non-pathogenic or genetically attenuated bacterial species capable of industrial or therapeutic use. These bacterial strains may be sourced from natural isolates, commensal microbiota, or environmental samples, and may be modified using synthetic biology or genetic engineering techniques to enhance production yield, stability, secretion efficiency, or safety. Additional genetic modifications may include the deletion or knockdown of undesirable genes—such as those associated with virulence, antibiotic resistance, or immune activation—to improve the safety, colonization efficiency, and regulatory compliance of the strain for use in therapeutic or supplemental applications. These bacterial systems may be utilized in live, inactivated, lysed, or cell-free formats, including the use of conditioned media or purified secreted products.

Accordingly, in various embodiments, the probiotic microorganism strains may be genetically engineered to optimize their ability to release the compound (i.e. TLR4 agonist) when administered and delivered in the gut of the subject. This may be achieved by overexpressing peptidoglycan hydrolases or autolysins, which may increase the release of the TLR4 agonist in the gut. For instance, in the case of Staphylococcus aureus, the compound may be secreted, with production enhanced by upregulating key enzymes such as muramidases and amidases involved in cell wall turnover and compound release. Advanced genetic modification tools, such as CRISPR-based genome editing or plasmid-based expression systems, may be employed to engineer biosynthetic pathways and transport mechanisms for the controlled production and secretion of the TLR4 agonist compounds.

In various embodiments, the probiotic microorganism may comprise a heterologous nucleic acid sequence encoding one or more biosynthetic enzymes or cell wall-modifying enzymes that enable the production in the microorganism, and/or secretion of the compound from the microorganism. In various embodiments, the probiotic microorganism may be modified to overexpress muramidases and/or amidases to enhance the release of the compound.

To ensure efficient delivery and colonization within the host gut, the engineered probiotic microorganism may be formulated into oral capsules, enteric-coated formulations, or fermented food products. Encapsulation techniques may be employed to protect the probiotics from gastric acidity and promote targeted release in the intestines. Upon reaching the gut environment, the engineered probiotics can colonize the intestinal mucosa and secrete the TLR4 agonist compounds through controlled autolysis, regulated secretion pathways, or fermentation-driven mechanisms. The released TLR4 agonist compounds subsequently interact with intestinal epithelial cells, macrophages, and dendritic cells, triggering TLR4-mediated immune activation and downstream immune-modulatory effects.

In various embodiments, the method may also include the co-administration of prebiotic substrates, which selectively enhance probiotic growth and release of the TLR4 agonist compounds. Additionally, regulatory elements may be incorporated into the engineered probiotics to allow inducible or condition-responsive secretion of TLR4 agonists, ensuring the therapeutic activity is activated only in response to specific gut conditions, such as inflammation or microbial imbalance.

The engineered probiotics microorganism may be administered in single or multiple doses, depending on the desired therapeutic or prophylactic outcome.

The method for using and/or engineering probiotic microorganisms provide a targeted, non-systemic, and host-friendly approach to modulating immune responses through probiotic-based delivery of the TLR4 agonist compound. By leveraging the natural colonization ability of probiotics, the method offers an efficient strategy for immunotherapy, adjuvant development, and gut microbiome engineering, thereby opening possibilities for treating and preventing immune-related gastrointestinal conditions.

In various embodiments, the efficacy of administering and treating a subject with the disclosed compound or composition may be monitored and assessed based on a comprehensive profile of gut microbiota-derived peptidoglycans (PGNs) in the subject. This profile can be generated using liquid chromatography-mass spectrometry (LC-MS), including high-resolution techniques such as LC-HRMS/MS, to provide a quantitative and qualitative analysis of soluble PGNs derived from the gut microbiota in the subject. The PGN profile may include an assessment of the concentration, composition, and structural variants of microbiota-derived PGNs, including the levels of the disaccharide GM (GlcNAc-MurNAc) in the analysed sample. Comparison of these levels against a reference or baseline profile may indicate treatment efficacy and/or predict the subject's responsiveness to the administration of the disclosed compound or composition. To ensure a comprehensive analysis, the sample used for profiling may be derived from various biological sources within the gastrointestinal (GI) tract, including but not limited to: Stool or fecal samples, providing a non-invasive and broad representation of gut microbial metabolites; Intestinal luminal contents, obtained through biopsy, endoscopic lavage, or intestinal aspiration, allowing for localized PGN analysis; Mucosal scrapings, collected from specific regions of the GI tract to assess PGN interactions with the host epithelium; Blood or serum samples, to detect circulating PGN metabolites as potential systemic biomarkers of gut microbiota activity; and/or Urine samples, to evaluate PGN metabolites that may be excreted and reflect microbial metabolism within the gut.

By integrating PGN profiling into treatment assessment, this approach enables a data-driven evaluation of gut microbiota interactions, providing insights into treatment efficacy, host immune modulation, and microbiome-related physiological changes in response to the administered compound or composition. In various embodiments, the sample may be a stool (fecal) sample that has been obtained from the subject.

In various embodiments, the reference or baseline profile may be derived from a reference sample or control sample obtained from the subject or another source such as a database, and refers to a standard, or level that is used for comparison purposes. In various embodiments, the reference or baseline profile is obtained from a healthy and/or non-diseased part of the body (e.g., tissue or cells) of the same subject or individual. In another embodiment, the reference or baseline profile is obtained from an individual who is not the subject or individual. In various embodiments, a decrease or reduction in the level of the disaccharide GM in the subject's gut relative to the reference profile, as indicated by the PGN profile, may suggest that the subject is suitable and will likely be responsive to administration of the disclosed compound and/or that treatment with the disclosed compound would be efficacious.

Accordingly, the methods of treating, preventing or ameliorating a subject disclosed herein, may further comprise a step of generating a PGN profile of the subject before and/or after administration of the compound. In various embodiments, the subject may be selected for administration of the compound based on the generated PGN profile. The generated PGN profile may provide a prediction or insight as to the responsiveness or non-responsiveness of the subject to treatment of the compound disclosed herein (i.e. assessment and/or monitoring of treatment efficacy).

It will be appreciated that while TLR4 agonists may have therapeutic applications, their utility extends beyond disease treatment, for example, when formulated as a supplement or nutritional composition, or a postbiotic in non-therapeutic uses intended to support normal gastrointestinal function in a healthy subject. As a non-therapeutic agent, a TLR4 agonist may help support and maintain gastrointestinal health by promoting balanced immune responses, reinforcing gut barrier integrity, and modulating the gut microbiota without inducing a therapeutic effect on a disease or pathological condition. These effects can contribute to overall digestive wellness, gut homeostasis, and resilience against environmental stressors, making TLR4 agonist-containing formulations valuable for individuals seeking to optimize gut health through dietary supplementation rather than pharmaceutical or therapeutic intervention. The compounds or compositions disclosed herein may be used, for example, during periods of dietary change, travel, aging, or environmental exposure to support optimal digestive system performance and resilience. These non-therapeutic effects contribute to maintaining a stable and healthy microbiota-host interface in the absence of disease or pathology.

Accordingly, the compounds or compositions or postbiotics may be used in non-therapeutic methods for promoting or maintaining gastrointestinal health in a subject comprising administering the compound or composition or postbiotic to the subject. The use of such compounds or compositions is intended to promote general health and well-being and does not include treatment or prevention of a specific disease. In such embodiments, the subject receiving the composition is preferably a healthy individual without a diagnosed gastrointestinal disorder or immune-mediated disease. In various embodiments, the subject is a “healthy subject” in such non-therapeutic methods.

In various embodiments of the non-therapeutic methods, the term “subject” generally has the same meaning as described above, but preferably relates to warm-blooded animal, preferably a mammal, more preferably a human that is healthy and is not considered to have a gastrointestinal condition or any condition or disease associated with aberrant TLR4 activity. All embodiments described above in relation to the therapeutic methods are similarly applicable to the non-therapeutic methods of the invention with the exception of the selection of the subject and absence of a related condition and disease that is to be treated.

The invention is further illustrated by the following non-limiting examples and the appended claims.

### Examples

### Materials and Methods

Human samples: Nanyang Technological University Institutional Review Board approval was obtained (IRB-2021-03-030) to collect human stool and serum samples from healthy participants. Informed consent was obtained from each volunteer. For study enrolment, volunteers from both genders need to meet the exclusion criteria as follows: 1) age between 22-65; 2) not pregnant; 3) not taking any antibiotic drugs during sample collection; and 4) no existing medical conditions. The serum was isolated and separated immediately after blood collection and aliquoted in Eppendorf tubes for storage at −80° C. Stool specimens were frozen at −80° C. and homogenized before PGN analysis.

Mice: Mice were age-, weight-, and sex-matched for all analyses. SPF TLR4/C57BL/6J mice (both genders) were purchased from Jackson Laboratory (USA) at 7-9 weeks of age for in-house breeding. WT C57BL/6J mice (both genders) were obtained from NTU animal facilities. Mice were housed under standard conditions in a climate-controlled environment with 12-hour light and dark cycles and fed ad libitum with a maintenance diet and water. Paired mice (1 male and 2 females per cage) were provided with a breeding diet. Mice were allowed to acclimate for 1 week prior to any studies. Male mice at 7-8 weeks of age were used in the DSS-induced colitis model and the antibiotic perturbation study. Animal experiments were approved by the Institutional Animal Care and Use Committee (IACUC) of NTU (Approval No: A23003 and A23039).

Cell culture: Caco-2 and RAW264.7 cells were obtained from the American Type Culture Collection. HEK293T cells were gifted by Prof. Anh Tuan Phan (NTU, Singapore). THP-1 cells were gifted by Prof. Veit Hornung (Ludwig Maximilian University, Germany). HEK-Blue™ Null1/Null2/hNod1/mNod1/hNod2/mNod2 cells were gifted by Prof Yue Wang (A*STAR Infectious Labs, Singapore). HEK-Blue™ mTLR4 cells, RAW-Dual™, and RAW-Dual™ KO-TLR4 cells were purchased from InvivoGen. RAW264.7 Myd88 KO cells were purchased from Ubigene. Caco-2 cells were maintained in high-glucose DMEM with sodium pyruvate (Gibco). RAW264.7 WT and Myd88 KO cells were cultured in high-glucose IMDM with HEPES (Gibco). THP-1 cells were maintained in RPMI 1640 (Gibco). All reporter cells (HEK-Blue™ mTLR4/Null1/Null2/hNod1/mNod1/hNod2/mNod2 cells, RAW-Dual™ and RAW-Dual™ KOTLR4 cells) were grown in high-glucose DMEM supplemented with 100 μg/mL Normocin (InvivoGen) and selection antibiotics as required. Specifically, HEK-Blue™ Null1 and Null2 cells were selected under 100 μg/mL Zeocin (InvivoGen); HEK-Blue™ mNod1, hNod1, mNod2 and hNod2 cells were selected under 100 μg/mL Zeocin (InvivoGen) and 30 μg/mL Blasticidin (InvivoGen); HEK-Blue™ mTLR4 cells were supplemented with 1×HEK-Blue™ Selection (InvivoGen). RAW-Dual™ and RAW-Dual™ KO-TLR4 cells were supplemented with 200 μg/mL Zeocin (InvivoGen) every other passage, but Zeocin was excluded during testing. Primary cells isolated from mice were sustained in IMDM with corresponding differentiation ligands. All abovementioned culturing media contain L-glutamine and were supplemented with 100 units/mL penicillin, and 100 μg/mL streptomycin (Cytiva) unless otherwise noted. DMEM and RPMI 1640 were mixed with 10% (v/v) heat-deactivated fetal bovine serum (FBS, Gibco) while IMDM was supplemented with 2% (v/v) heat-inactivated FBS instead. Cells were routinely cultured in FBS- and antibiotics-supplemented complete media at 37° C. with 5% CO2.

Human serum and stool collection: The freshly collected blood was kept still for 15 min at room temperature for spontaneous agglutination, followed by centrifugation at 3,000 rpm for 15 min with a low accelerating and decelerating speed at 4° C. The serum at the top layer was collected and aliquoted into new 1.5 mL tubes for sample cleanup for LC-MS analysis directly or stored at −80° C. For optimal serum PGN detection by LC-MS, it is recommended performing serum protein removal within the same day of blood collection. It was found that PGN signals gradually decreased in serum samples that were frozen immediately upon collection without protein removal processing.

Sterile stool sample collection kits were distributed to the volunteers within the week of blood donation. Stool samples were weighed and resuspended in sterile H₂O followed by homogenization aided by autoclaved stainless-steel beads using a PowerLyzer 24 Homogenizer (Qiagen) at 1,000 rpm for 1 min. The homogenized stool samples were centrifuged at 12,000 rpm for 5 min, and the supernatant was collected into new tubes. The reminder stool pellet was further resuspended with sterile H₂O by vortexing for 30s followed by high-speed centrifugation (12,000 rpm for 5 min). The supernatant was combined and diluted to a final concentration of 50 mg stool per 1 mL sterile H₂O. The combined stool supernatant was subjected to centrifugation at 16,000×g for 5 min to remove any debris carried over from the previous steps. The stool supernatants (50 mg/mL) were saved at −80° C. as aliquots for further analysis.

Serum and stool sample cleanup: For MurA quantification by LC-HRMS/MS, 100 μL conc. HCl (12 M stock) was added to a 200 μL sample (fecal supernatant or standard MurNAc) such that the final concentration of HCl in the mixture was 4 M. The mixture was subjected to acid hydrolysis by heating at 90° C. for 3 h, followed by acid removal by a rotatory evaporator. The concentrated samples were reconstituted in 1% (v/v) TFA in dH₂O and were subjected to cleanup with a pre-equilibrated Strata C18-E solid phase extraction (SPE) cartridge (Phenomenex), where the elute fractions of 0-20% acetonitrile in water were collected, lyophilized, and resuspended in 200 μL dH₂O. Upon centrifugation at 16,000×g for 5 min to remove any sediments, the clarified sample was collected for MurA quantification. For the serum sample (1 mL), methanol (9 mL, HPLC grade) was added to precipitate serum proteins such as albumin. After centrifugation at 16,000×g for 5 min, the clarified serum supernatant was collected, dried by rotatory evaporation, and reconstituted in 200 μL dH₂O (i.e. 5× concentrated than the original serum), which was then subjected to acid hydrolysis as described above. Upon SPE cleanup, the dried serum sample was resuspended in 100 μL dH₂O for MurA quantification by LC-HRMS/MS (i.e. the final conc is 10× of the original serum). It was found that concentrating serum samples by 10× is necessary to enhance MurA signals in LC-MS analysis. To note, FBS from Capricorn (Cat. No. S1810) and Biowest (Cat. No. FBS-11A) was subjected to MurA analysis following the same protocols.

For PGN subtype profiling by LC-MS, 1 mL fecal supernatant (50 mg/mL) was directly subjected to SPE cleanup, where elute fractions of 0-20% acetonitrile in water were collected, lyophilized, and resuspended in 100 μL dH₂O for LC-HRMS/MS analysis (i.e. the final concentration is 10× the original sample). 1 mL of serum was subjected to protein precipitation with methanol (9 mL) before SPE cleanup. The collected elute fractions from the SPE column were dried and reconstituted in 100 μL dH₂O for LC-HRMS/MS analysis (i.e. the final concentration is 10× the original samples). It was found that sample concentration was necessary for PGN subtype analysis. For quantification of PGN subtypes, 1 μg/mL sialic acid (Sigma-Aldrich, Cat. No. A0812) was spiked into the final reconstituted samples as an internal standard in LC-MS analysis.

Beta-lactam treatment to mice: The protocol was adopted from a previous study. [16] Briefly, 0.5 mL of ampicillin (8 mg/mL) was orally gavaged into mice (n=8-10). Fecal samples were collected at different time points postgavage (i.e., Oh, 6 h, 24 h, and 48 h). Mice were sacrificed by euthanasia at the end time point to harvest ceca. Feces and ceca were weighed and homogenized to 50 mg/ml in sterile dH₂O. Supernatants after high-speed centrifugation were stored at −80° C. for further analysis.

LC-MS/MS workflow for PGN quantification and profiling: PGN analysis was performed with the Vanquish HPLC system equipped with Orbitrap Exploris 120 mass spectrometer (Thermo Fisher Scientific). The autosampler was controlled at 18° C. and the column oven was at room temperature, with sample injection volume set as 10 μL sample for each run. Gradient separation was achieved with a Phenomenex C18 Kinetex EVO18 RP column (2.0 mm×150 mm, 100 A) with solvent A (0.05% formic acid in H₂O, LC/MS grade, Fisher Scientific) and solvent B (0.05% formic acid in acetonitrile, LC/MS grade, Fisher Scientific) at a constant flow rate of 0.2 mL/min. For MurA quantification, the LC program starts with 1% B holding for 2 min, followed by a linear gradient to 30% B at 5 min, then up to 90% B at 6 min, which was held for 2.5 min. The solvent gradient was then converted to 1% B within 0.1 min and kept for 2.4 min as pre-equilibration for the next run. For PGN subtype profiling, the LC method starts with 1% B for 2.5 min, followed by a steady linear increase to 40% B at 27.5 min, which was kept for 2.5 min. Next, the gradient increases to 90% B at 31 min, which was held for 4 min followed by the return to 1% B at 36 min and kept for 4 min till the end as pre-equilibration for the next run. Elutes of the first minute were diverted to waste to avoid solvent peak suppression and high salt contamination.

The OT Exploris 120 mass spectrometer was operated in positive ion mode using electrospray ionization (ESI) and internal calibration. The spray voltage was 3500 V with a vaporizer temperature of 300° C. and an ion transfer tube temperature of 325° C. Gas was statically flowing at 50, 10, and 0 (Arb) for sheathing, auxiliary, and sweeping gases, respectively. For MurA quantification, only MS2 spectra were acquired in parallel reaction monitoring (PRM) mode for the target precursor ion at m/z=252.1078. MS1 spectra were collected in full-scan (FS) mode, ranging from 120-1,200 m/z with a resolution of 60,000. MS2 spectra were acquired for the fragmentation of assigned precursors in full-scan data-dependent analysis (FSDDA) mode when the intensity was above the threshold of 1.0e5. For the acquisition of MS2 spectra, 4 MS/MS scans were acquired for each precursor ion (within the isolation window of ±1 m/z) with a higher energy collision dissociation (HCD) energy of 30% and a resolution of 15,000.

Cell culture and PGN treatment: RAW264.7, RAW-Dual™, and RAW-Dual™ KO-TLR4 cells were harvested and seeded as 100,000 cells/well in 24-well plates for 24 h before PGN treatment. THP-1 cells were harvested and seeded as 100,000 cells/well in 24-well plates and immediately proceeded with ligand stimulation. 1 mL of complete media was added to each well without any selection antibiotic. HEK-Blue™ Null1/Null2/hNod1/mNod1/hNod2/mNod2 cells were seeded as 40,000-50,000 cells/well in flat-bottom 96-well plates and used immediately for PGN stimulation.

HEKBlue™ mTLR4 cells were seeded as 25,000 cells/well in flat-bottom 96-well plates and used immediately for PGN stimulation. 0.2 mL of complete media containing selection antibiotics were added in each well.

Cells were stimulated with commercially available ligands or chemically synthesized PGNs (i.e., saccharides and their analogues). Compounds were either directly added or premixed with transfection reagent Lipofectamine™ 3000 (Invitrogen™, Cat. No. L3000015) in serumfree media before addition. Cells were incubated with ligands for various amounts of time, depending on the assays. The specific ligand concentration and incubation time were indicated in figure legends. Upon ligand stimulation, cells were harvested for western blot, RTqPCR, or RNAseq analysis. Culture supernatants were collected for ELISA or reporter assay analysis. Ligands and small molecule inhibitors used in cell treatment are listed in Table 1.

TABLE 1
Commercial compounds used in cell treatment.

| | | |
|---|---|---|
| M | Sigma-Aldrich | Cat. No. M2503 |
| Muramyl dipeptide | MedChemExpress | Cat. No. HY-127090 |
| Di-acetyl chitobiose | Sigma-Aldrich | Cat. No. SMB00279 |
| Chitobiose | This study | QY lab, NTU |
| Lipopolysaccharide (E. coli | InvivoGen | Cat. No. tlrl-peklps |
| K12) | | |
| Lipoteichoic acid (S. aureus) | Sigma-Aldrich | Cat. No. L2515 |
| Poly (I:C) LMW | InvivoGen | Cat. No. tlrl-picw |
| CpG-B | InvivoGen | Cat. No. tlrl-1668 |
| TAK-242 | MedChemExpress | Cat. No. HY-11109 |
| AT791 | MedChemExpress | Cat. No. HY-124603 |
| CU-CPT22 | MedChemExpress | Cat. No. HY-108471 |
| CU-CPT 4a | MedChemExpress | Cat. No. HY-108473 |
| Polymyxin B sultfate | Sigma-Aldrich | Cat. No. P4932 |
| Polymyxin B agarose beads | Sigma-Aldrich | Cat. No. P1411 |
| Lipofectamine ™ 3000 | Invitrogen | Cat. No. L3000015 |
| Transfection Reagent | | |

Murine bone marrow cell isolation and differentiation: Femur and tibia were harvested from euthanized SPF C57BL/6J female mice. Both ends of the bone were cut off and 10 mL complete media were used to flush a pair of femur and tibia through a 27G needle. Cells were centrifuged at 300×g for 5 min and then resuspended in red blood cell lysis buffer (0.89% NH₄Cl, w/v, 10 mM KHCO₃, 0.1 mM EDTA) for 8 min. Lysis was quenched by complete media. White blood cell pellets were resuspended in complete media followed by filtration and counting.

Cells were diluted to 2,000,000 cells/mL in BMDC media (complete media supplemented with FLT3 ligand-containing cell supernatants) and seeded as around 20,000,000 cells in a 10 cm culturing dish for dendritic cell differentiation. 10 mL complete media were added to the same dish on day 3 and day 6. DCs were harvested on day 9 and 2,000,000 cells/well were seeded in a 24-well plate. 1 mL of BMDC media was added to each well.

Cells were diluted to 200,000 cells/mL in BMDM media, complete media supplemented with 20 ng/ml recombinant GM-CSF (Biolegend, Cat. No. 576306), for macrophage differentiation. 40,000 cells/well were seeded in a flat-bottom 96-well plate for 6 days. 0.2 mL of complete media was added to each well. The media was replaced by freshly prepared BMDM media on day 3. Cells were ready by day 6.

Reporter cell assay: 20 μL of supernatants from treated reporter cells were mixed with 180 μL Quanti-Blue reagent (InvivoGen) and incubated at 37° C. for 6 h. The absorbance was measured at λ=620 nm after 10s shaking by a Thermo Multi-Scanner Microplate reader. The readouts were blanked with negative control (i.e. sterile dH₂O) and also subtracted by the readouts of the respective parental cell line (if any).

20 μL of supernatants from treated reporter cells were mixed with 50 μL of Quanti-Luc 4 reagent (InvivoGen) and luminescence was detected immediately by CYTATION5 imaging reader (BioTek) and recorded by Gen3.2 software. Auto gate and default luminescence filter were set for recording.

RNAseq and RT-qPCR analysis: The total RNA from cells was extracted by Monarch Total RNA Miniprep Kit (New England Biolabs) according to the vendor's recommended protocols. Tissues were first homogenized in TRIzol reagents (Ambion, Invitrogen) and stored at −80° C. until further processing.

Chloroform was added to the TRIzol-tissue complex and sat for 5 min after vortexing followed by high-speed centrifugation (16,000×g for 10 min) at 4° C. The top layer containing RNA/DNA was separated. Total tissue RNA was extracted by the same kit according to the recommended protocols. RNA was eluted by RNase-free ddH₂O, and its concentration was determined by NanoDrop™ One Microvolume UV-Vis Spectrophotometer (Thermo Fisher Scientific). Samples were preserved at −80° C. for RNAseq (Novogene) or RT-qPCR analysis. RNA (500-1,000 ng) was utilized for reverse transcription of each sample (normalized within each batch) by ProtoScript II First Strand cDNA synthesis kit (New England Biolabs) or MMLV reverse transcriptase (Promega) to yield cDNA according to the recommended protocols. Necessary reagents including Oligo-dT (5′-TTT TTT TTT-3′, 100 μM in IDTE buffer, Integrated DNA Technologies), dNTP mix (0.5 mM, Thermo Fisher Scientific), RiboLock RNase Inhibitor (1 U/μL, Thermo Fisher Scientific) were mixed with M-MLV reverse transcriptase as required. cDNA samples were then diluted if needed or directly used as templates for quantitative PCR (YouSeq Tetra™ OneStep 2× qRT-PCR Mastermix or Bio-Rad SsoAdvanced Universal SYBR Green Supermix). The readouts were obtained from the Eco Real-Time PCR System (Illumina) or CFX Duet Real-Time PCR System (Bio-Rad). Relative gene expression of target genes was determined utilizing 2^{-ΔΔCt} method and normalized to the housekeeping gene act-b. Primers used (customized design and ordered from Integrated DNA Technologies) are listed in Table 2.

TABLE 2
Primers used

| Primer (Mus | | SEQ ID |
|---|---|---|
| musculus) | Sequence (5′ → 3′) | NO: |
| Actb_fw | CACTGTCGAGTCGCGTCC | 1 |
| Actb_rv | TCATCCATGGCGAACTGGTG | 2 |
| Tnfa fw | GTCCCCAAAGGGATGAGAAGTT | 3 |
| Tnfa_rv | CTCCTCCACTTGGTGGTTTG | 4 |
| Il1a_fw | CCCATGATCTGGAAGAGACCA | 5 |
| Il1a_rv | CAAACTTCTGCCTGACGAGC | 6 |
| Il1b_fw | TGCCACCTTTTGACAGTGATG | 7 |
| Il1b_rv | TGATGTGCTGCTGCGAGATT | 8 |
| Il12a_fw | ACGAGAGTTGCCTGGCTACTAG | 9 |
| Il12a_rv | CCTCATAGATGCTACCAAGGCAC | 10 |
| Il12b_fw | AGACCCTGCCCATTGAACTG | 11 |
| Il12b_rv | GGCGGGTCTGGTTTGATGAT | 12 |
| Ccl5_fw | CACCATATGGCTCGGACACC | 13 |
| Ccl5_rv | TCGAGTGACAAACACGACTG | 14 |
| Cxcl10_fw | CCACGTGTTGAGATCATTGCC | 15 |
| Cxcl10_rv | TCACTCCAGTTAAGGAGCCC | 16 |
| Il6_fw | CGGCCTTCCCTACTTCACAA | 17 |
| Il6_rv | TGCCATTGCACAACTCTTTTC | 18 |
| Ccl2_fw | CACTCACCTGCTGCTACTCA | 19 |
| Ccl2_rv | GCTTGGTGACAAAAACTACAGC | 20 |
| Il27_fw | TGTCCACAGCTTTGCTGAAT | 21 |
| Il27_rv | GAAGTGTGGTAGCGAGGAAGC | 22 |
| Ifnb1_fw | CGTGGGAGATGTCCTCAACT | 23 |
| Ifnb1_rv | AGATCTCTGCTCGGACCACC | 24 |
| Keap1_fw | ACAGCAGCGTGGAGAGATATG | 25 |
| Keap1_rv | GTTAAGCCGGTTAGTCCCGT | 26 |
| Hspb1_fw | TCACCCGGAAATACACGCTC | 27 |
| Hspb1_rv | GGCCTCGAAAGTAACCGGAA | 28 |
| Il10 fw | GCTGTCATCGATTTCTCCCCT | 29 |
| Il10_rv | GACACCTTGGTCTTGGAGCTTAT | 30 |
| Tlr4_fw | TCCCTGCATAGAGGTAGTTCC | 31 |
| Tlr4_rv | TCAAGGGGTTGAAGCTCAGA | 32 |
| Nod2_fw | TGTCCAACAATGGCATCACCT | 33 |
| Nod2_rv | TGTGTTCCCTCGAAGCCAAA | 34 |
| Primer (Homo | | |
| sapiens) | Sequence (5′ → 3′) | |
| Actb_fw | CTCGCCTTTGCCGATCC | 35 |
| Actb_rv | TCTCCATGTCGTCCCAGTTG | 36 |
| Tnfa_fw | TGGGATCATTGCCCTGTGAG | 37 |
| Tnfa_rv | GGTGTCTGAAGGAGGGGGTA | 38 |
| Il8_fw | ACCGGAAGGAACCATCTCAC | 39 |
| Il8_rv | GGCAAAACTGCACCTTCACAC | 40 |
| Primer | Sequence (5′ → 3′) | |
| mTLR4-K263A-F | ATCTTGGGAGAATTTGCTGATGAAAGGAATCTG | 41 |
| mTLR4-K263A-R | CAACCGATGGACGTGTAAACCAGCCAGGTTTTG | 42 |
| mTLR4-Q339A-F | CATTAGATGTGCTCTTAAGCAGTTTCCAAC | 43 |
| mTLR4-Q339A-R | ATTGATAAGGATTGCCATTTGAAATGTTTAG | 44 |
| mMD2-F119A-F | CATCAATACCAGCTTCTTTCGAGGGAATAC | 45 |
| mMD2-F119A-R | TATTCACAGTCTCTCCTTTCAGAGCTCTGC | 46 |
| mMD2-F126A-F | CGAGGGAATACTAGCTCCTAAGGGCCATTAC | 47 |
| mMD2-F126A-R | AAAGAGAATGGTATTGATGTATTCACAGTC | 48 |

ELISA: All samples used for ELISA were centrifuged at 16,000×g for 5 min to remove any potential sediments or cell debris right before analysis.

IcELISA for PGN quantification was performed as reported. [16] 96-well half-area plates were used (Costar, Corning). Serial dilutions of MDP were used to generate a calibration curve for icELISA in each plate. The final absorbance at λ=492 nm was recorded by Tecan Infinite 200 Pro (Tecan Life Sciences).

ELISA MAX™ standard sets (Biolegend) were utilized to evaluate cytokine or chemokine (human IL-6, TNF-α, IL-8 and mouse IL-6, TNF-α, and MCP-1) in culture supernatants following the manufacturer's recommended protocols. Samples were prediluted with blocking buffer (1% BSA in PBS, pH 7.4), if required. BSA (Bovine Serum Albumin, powder, Sigma-Aldrich) stock solution (1%, w/v) was dissolved in sterile PBS (pH 7.4) followed by filtration. The substrate TMB set (Biolegend) was added, and the reaction was stopped by 1M sulfuric acid. The final absorbance at λ=450 nm was recorded by Thermo Multi-Scanner Microplate and SkanIt software (Thermo Scientific).

Molecular docking of GM into murine TLR4-MD2 model: The chemical structure of GM was converted from its SMILES notation [CC(═O)NC1C(OC2C(CO)OC(O)C(NC(C)═O)C2OC(C)C(═O)O)OC(CO)C(O)C1O] to PDB file using OpenBabel. The model of ligand-free murine TLR4-MD2 protein complex was obtained from UniProt (PDB: 5IJB). [17] To generate the ligand and receptor for docking, water and other molecules were removed from each of the GM and mTLR4-MD2 models and converted to individual PDBQT files. Docking simulation was conducted using AutoDock-Vina by fitting GM into the reported LPS binding site of mTLR4-MD2 in a 30×30×30 A grid box. The docked complex with the lowest free-binding energy was selected for visualization and analysis using BIOVIA Discovery Studio.

Murine TLR4-MD2 co-transfection in HEK293T and PGN treatment: Recombinant plasmid pFlag-CMV-1::mTLR4 harbouring FLAG-tagged murine toll-like receptor 4 (mTLR4) cDNA was obtained from Addgene (Cat. No. 13087). c-Myc-tagged murine MD2 was de novo synthesized as gBlock fragment (Integrated DNA Technologies) according to the cDNA sequence from NCBI (accession number NM_016923) and cloned into pcDNA3.1-Zeo backbone between KpnI and EcoRI sites, yielding pcDNA3.1-Zeo: mMD2-Myc. E. coli DH5α was transformed and colonies were selected with carbenicillin. mTLR4-MD2 mutants were generated according to the Q5 site-directed mutagenesis kit (New England Biolabs) using SDM primers (Table 3). Plasmid was extracted and purified using FavorPrep™ Plasmid DNA Extraction Kit (Favorgen Biotech Corp.) according to the manufacturer's protocol.

TABLE 3
Primers used in murine TLR4-MD2 mutagenesis.

| Primer | Sequence (5′ → 3′) | SEQ ID NO: |
|---|---|---|
| mTLR4-K263A-F | ATCTTGGGAGAATTTGCTGATGAAAGGAATCTG | 49 |
| mTLR4-K263A-R | CAACCGATGGACGTGTAAACCAGCCAGGTTTTG | 50 |
| mTLR4-Q339A-F | CATTAGATGTGCTCTTAAGCAGTTTCCAAC | 51 |
| mTLR4-Q339A-R | ATTGATAAGGATTGCCATTTGAAATGTTTAG | 52 |
| mMD2-F119A-F | CATCAATACCAGCTTCTTTCGAGGGAATAC | 53 |
| mMD2-F119A-R | TATTCACAGTCTCTCCTTTCAGAGCTCTGC | 54 |
| mMD2-F126A-F | CGAGGGAATACTAGCTCCTAAGGGCCATTAC | 55 |
| mMD2-F126A-R | AAAGAGAATGGTATTGATGTATTCACAGTC | 56 |

Variant mTLR4-mMD2 complex was co-transfected into HEK-Blue™ Null1 (seeding density: 50,000 cells/mL complete media, 1 mL/well in 24-well plate) using Lipofectamine 3000 according to the manufacturer's protocol. In each well, cells were transfected with a total of 500 ng of pFlag-CMV-1::mTLR4 and pcDNA3.1-Zeo::mMD2-Myc (mixed at 1:1) for 16 h. Water-Lipo mixture instead of plasmids was added as mock-transfected cells. Culture media was replaced with fresh complete DMEM the next day, and the transfected cells were then incubated with various PGN compounds for 24 h. Culture supernatants were collected to quantify cytokine production by ELISA and to measure NF-κB activity based on SEAP-reporter assay.

Immunoblotting: RAW-Dual™ and RAW-Dual™ KO-TLR4 cells directly stimulated with commercially available ligands or chemically synthesized GM for 4 h were harvested. Cells were boiled in 1×SDSloading buffer supplemented with 50 mM DTT for 15 min followed by SDS-PAGE and PVDF membrane transfer. The membranes were blocked with 3% skim milk in TBST at room temperature for 1 h. Each membrane was incubated with primary antibodies (dilution factor 1:1000) corresponding to rabbit anti-mouse GAPDH or total/phosphorylated NF-κB p65, MAPK p38, and TBK1 (Cell Signalling Technology) at 4° C. overnight after washing. The secondary antibody (HRP goat anti-rabbit, Invitrogen) was added to each membrane (dilution factor 1:5000) and incubated for 1 h at room temperature after washing. For all washing steps, the membrane was rotated in 10 mL TBST for 5 min for three rounds. After the final washing steps, Amersham™ ECL Select™ detection reagent (Cytiva) was added to the membranes, and protein bands were visualized and documented using ImageQuant 800 (Cytiva).

mTLR4 pulldown by immobilized GM-agarose beads: Recombinant mouse TLR4 (26-638)-His6 protein (Cusabio, Cat. No. CSB-YP023603MO) was reconstituted in water and aliquoted for storage at −80° C. 40 μL alkyne agarose beads (Vector laboratories, Cat. No. CCT-1032) were washed with 400 μL dH₂O twice before usage. Washed beads were collected by centrifugation at 2800 rpm for 45 sec. To immobilize GM onto the beads, GM-1-N3 (2.5 mM) was added together with 1.2 mM sodium ascorbate, 0.5 mM CuSO4, and 0.5 mM THPTA to a total volume of 200 μL for click reaction at 4° C. for 3 h with rotation. For negative control, click reagents were added but without the GM-1-N3 probe. Following click reactions, the GM-immobilized agarose beads and control alkyne-agarose beads were washed, and incubated with recombinant mTLR4 (26-638)-His6 protein (100 ng or 50 ng) in a total volume of 200 μL in dH₂O for overnight incubation at 4° C. The beads were centrifuged (at 2800 rpm for 45 sec) and the supernatant was collected as flow-through (FT), washed with 400 μL dH₂O (wash), and subjected to boiling in 40 μL 5×SDS-loading buffer containing 50 mM DTT for 15 min (elute). 20 μL boiled samples were loaded onto SDS-PAGE gel for analysis. For Western blot analysis, the transferred PVDF membrane was blocked with 5% skim milk in TBST at room temperature for 1 h, washed (10 mL TBST for 5 min×3), and incubated with HRP-conjugated anti-His Ab (H-3, 1:1000 dilution, Santa Cruz Biotechnology) for 1 h at room temperature. After final washing (10 mL TBST for 5 min×3), the Amersham™ ECL Select™ detection reagent (Cytiva) was added onto the membrane for visualization with ImageQuant 800 (Cytiva).

Dextran sulfate sodium (DSS)-induced colitis model: 3% DSS (MP Biomedicals) drinking water (w/v) was given to SPF C57BL/6J male mice for continuous 7 days followed by euthanasia. GM (10 mg/kg, 0.1 mL final volume) or PBS (0.1 mL) was intraperitoneally injected into mice daily starting from 2 days before the DSS water administration until the end of experiments. The body weight of each mouse was recorded daily.

Colon immune cell isolation: Colon immune cells were isolated following the reported protocol with minor modifications. Generally, the colon was harvested, cut open, and rinsed with cold PBS (pH 7.4) to remove the luminal contents. To remove the epithelium, the colon was incubated in 15 mL complete HBSS (Hanks's balanced salt solution without Ca2+ and Mg2+ supplemented with 1.3 mM EDTA and 0.35 g/L sodium bicarbonate, Sigma-Aldrich) at 37° C. for 20 min with shaking. The buffer was replaced with another 15 mL of fresh complete HBSS for vortexing for 25 min at 37° C. The colon was then washed with IMDM complete media and then cut into small pieces, which were subsequently digested in IMDM complete media containing 1 mg/mL collagenase D (Roche) and 20 U/mL DNase I (Life Technologies) at 37° C. for 70 min. The digested tissue was then gently mashed through a 100 μm cell strainer. The leukocytes were enriched using a 70%-30% Percoll gradient (GE Healthcare Life Science), where cells at the interface were collected and stained for flow cytometry analysis.

Flow cytometry: Cells collected from in vitro culture or isolated from mice were suspended in flow solution (PBS+2% FBS). Cells were stained and subsequently analyzed using a BD LSRFortessa or FACSymphony A3 five laser flow cytometer (BD Biosciences). Data were analyzed using FlowJo software (TreeStar). Cells were blocked with a homemade Fc-blocker (1:350 dilution). BMDCs were stained with antibodies with indicated dilution factor as listed for 20 min: anti-MHC-II (Biolegend, Cat. No. 107632), anti-SiglecH (Biolegend, Cat. No. 129604), anti-CD11c (Biolegend, Cat. No. 117318), anti-CD11b (Invitrogen, Cat. No. 45-0112-82), anti-CD103 (Invitrogen, Cat. No. 17-1031-82), anti-CD80 (Biolegend, Cat. No. 104708) and anti-CD86 (Biolegend, Cat. No. 105008), followed by Live/Dead Ghost dye Violet 510 (Tonbo Biosciences Cat. No. 50-105-2993) 1:1000 diluted in PBS for 5 min. Cells isolated from the colon were stained were stained with antibodies with indicated dilution factor as listed for 20 min: anti-CD45 (BD biosciences, Cat. No. 748371), anti-MHC-II (Biolegend, Cat. No. 107639), anti-CD11c (Biolegend, Cat. No. 117318), anti-CD11b (Biolegend, Cat. No. 101226), anti-CD64 (Biolegend, Cat. No. 139306), Ly6C (Biolegend, Cat. No. 128006), Ly6G (BD biosciences, Cat. No. 563978), and F4/80 (Biolegend, Cat. No. 123110), followed by DAPI (Biolegend, Cat. No. 105008) 1:1000 diluted in PBS for 5 min. Cells were finally resuspended in flow solution before subjection to flow cytometry for analysis.

Inflammatory panel of mouse sera was evaluated according to the manufacturer's protocol. The beads were finally reconstituted in the washing buffer offered in the kit and analyzed using FACSymphony A3 five laser flow cytometer (BD Biosciences) attached to a high throughput sampler (BD Biosciences). Data were uploaded and analyzed using the corresponding software QOGNIT (Biolegend).

Histology: The colon was separated immediately after the scarification of mice. The lumen was cut open and flushed with PBS (pH 7.4, on ice) to remove excretions. The colon was further washed with cold PBS (pH 7.4) for 5 rounds with vortexing for 10s per round. The clean colon was dipped into ethanol glacial acetic acid fixative for at least 30 min. Ethanol and acid were washed away by 1×PBS (pH 7.4). The colon was rolled and placed in the cassette in 4% (w/v) paraformaldehyde (PFA) solution diluted from stock (10% w/v aq. soln., methanol free, Thermo Scientific) for long-term preservation. The samples were sent to the A*STAR, AMPL for histology service, where paraffin block preparation, slide preparation, and Hematoxylin & Eosin staining were completed. Three sections at different depths were collected on a slide for each sample. The images were taken by Nikon Eclipse 80i at magnification of 4× and 20× respectively with default auto white compensation during imaging.

Statistical analysis: Graphs were prepared with statistical analysis using GraphPad Prism 9. Results were illustrated as mean values±the standard error of the mean (SEM) unless otherwise noted. Statistical analysis was performed using one-way analysis of variance (ANOVA) followed by Dunnett's multiple comparisons tests, or two-way ANOVA followed by Tukey's multiple comparison tests wherever appropriate. The statistical tests used, significance according to p-values, and sample sizes were indicated in each figure legend. Data were representative of two or three independent experiments with minimum duplicate measurements.

General Synthetic Procedures: Water was purified with a Millipore Milli-Q system (Merck K. Ga. Co., Darmstadt, Germany). Chemical reagents and solvents were obtained from commercial sources (Millipore-Sigma, TCI, Alfa-Aesar, Fluorochem, or BLDPharm). Cold temperatures were maintained using the following conditions: 0-5° C., ice-water bath; −78° C., acetone-dry ice bath. Normal phase column chromatography was carried out with Grace Davisil® LC60A 40-63 micron silica gel. Preparative HPLC was performed on an Agilent 1260 Infinity II equipped with a 21.2×250 mm C18 column using a 10 mL/min flow rate. Removal of solvents was done with Buchi R-100 rotary evaporator equipped with a Vacuubrand® MD-1C diaphragm vacuum pump and an Eyela CCA-1110 chiller. Final muropeptide products were lyophilized from water/acetonitrile to ensure an accurate weight.

NMR spectra were recorded on a Bruker Avance 400 spectrometer (400 MHz for ¹H NMR, 101 MHz for ¹³C NMR). Spectral measurements of muropeptide final products were performed using a Shigemi BMS-005B NMR tube. Spectra were recorded using CDCl₃, DMSO-d₆, or D₂O. ¹H and ¹³C signal positions (δ) are reported in parts per million from tetramethylsilane (δ 0) and were measured relative to the signal of the solvent (¹H NMR: CDCl₃ δ 7.26, DMSO-d₆ δ 2.50; ¹³C NMR: CDCl₃ δ 77.16, DMSO-d₆ δ 39.52). When D₂O was used, about 0.2 μL DMSO was spiked in as an internal standard (δ 2.71 for ¹H NMR and δ 39.39 for ¹³C NMR). Coupling constants (J values) are reported in Hertz (Hz). ¹H NMR spectral data are tabulated in the order: multiplicity (s, singlet; d, doublet; t, triplet; q, quartet; m, multiplet; br., broad), coupling constant, number of protons. Only selected diagnostic peaks are assigned. In cases where corresponding signals of the alpha and beta isomers have very close chemical shifts, they are not assigned separately. For example, H-4α and H-4β are tabulated as H-4. Glycan rings are annotated as follows.

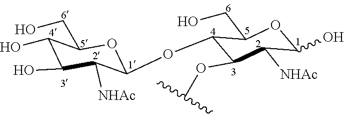

High-resolution mass spectra were measured using a Themo-Fisher Vanquish Core HPLCOrbitrap Exploris 120 system.

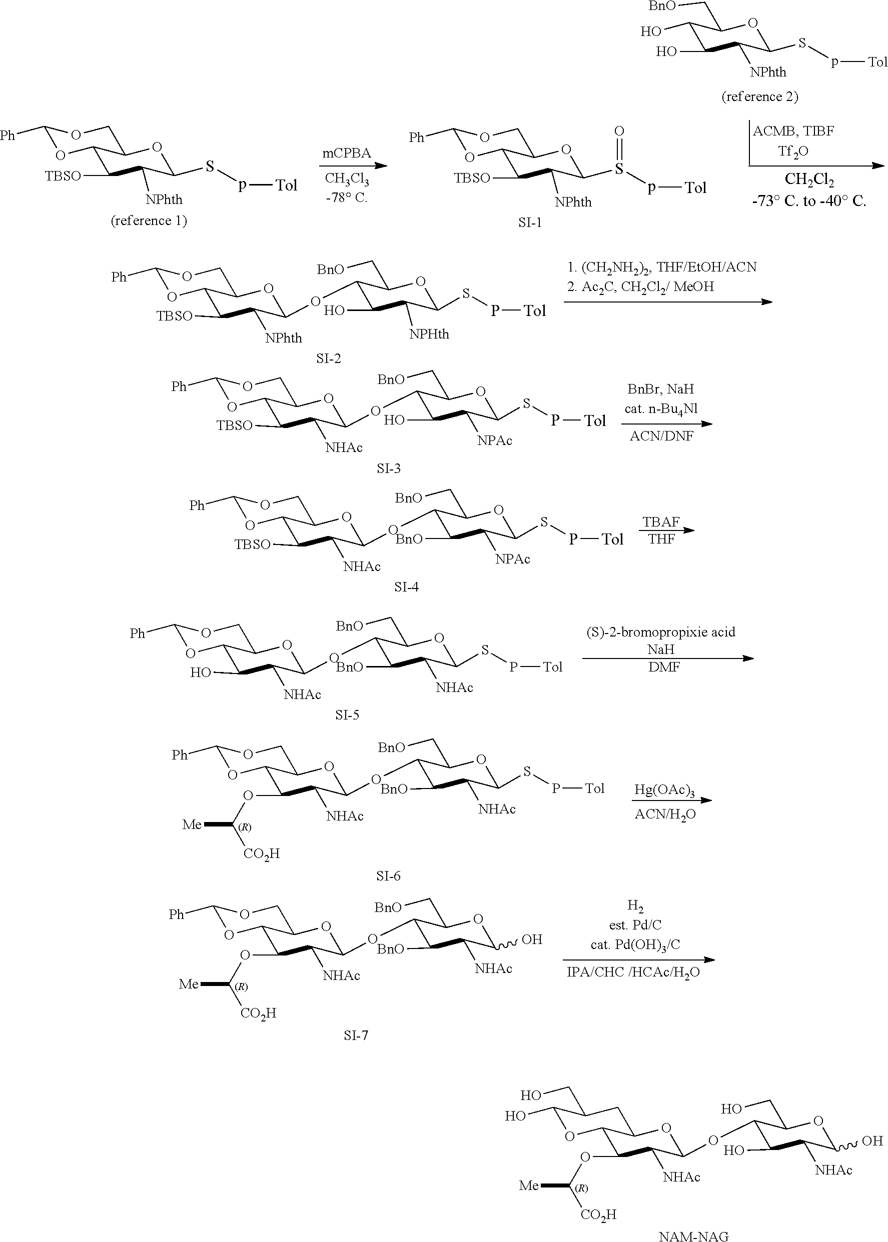

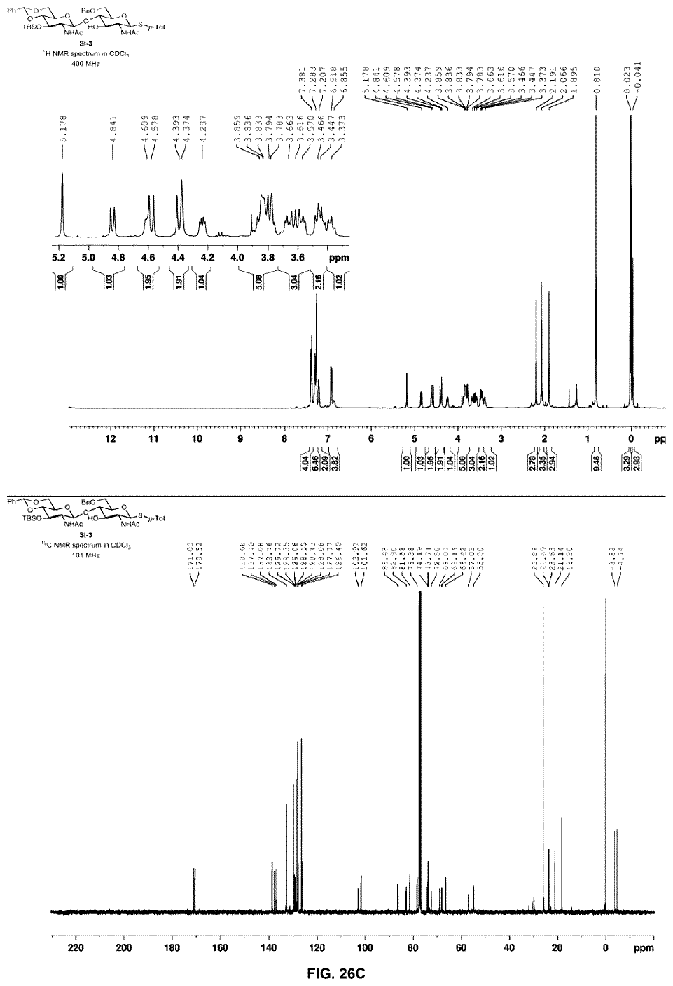

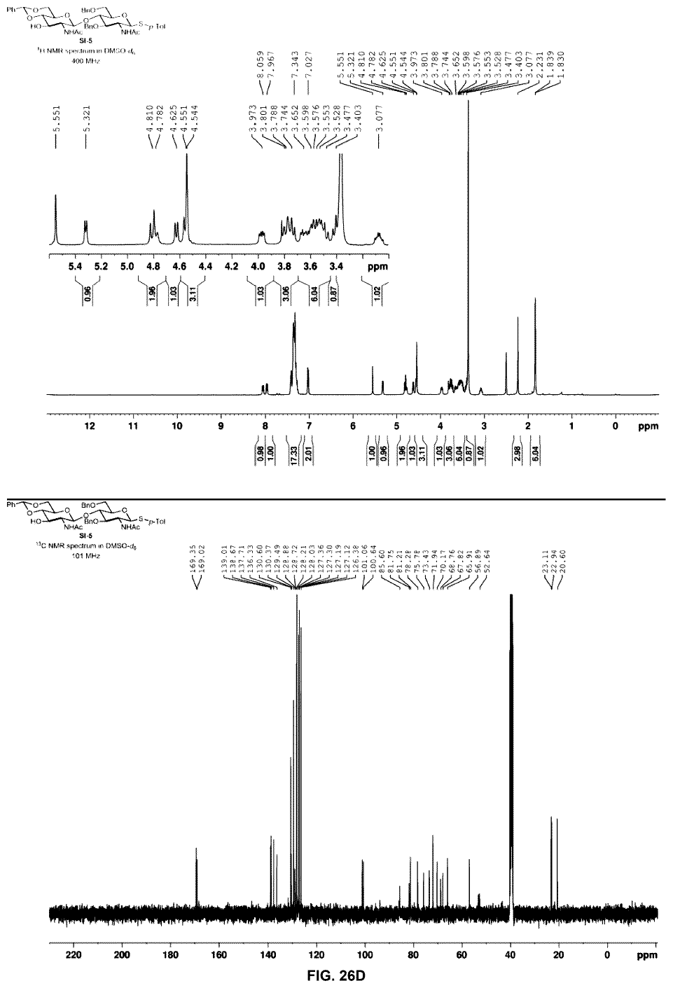

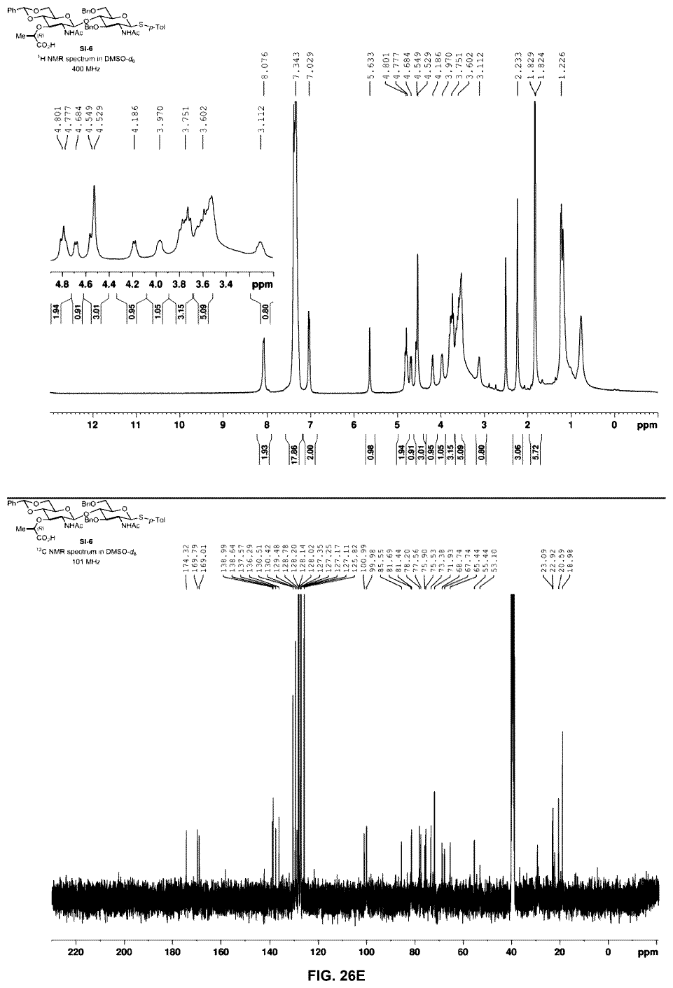

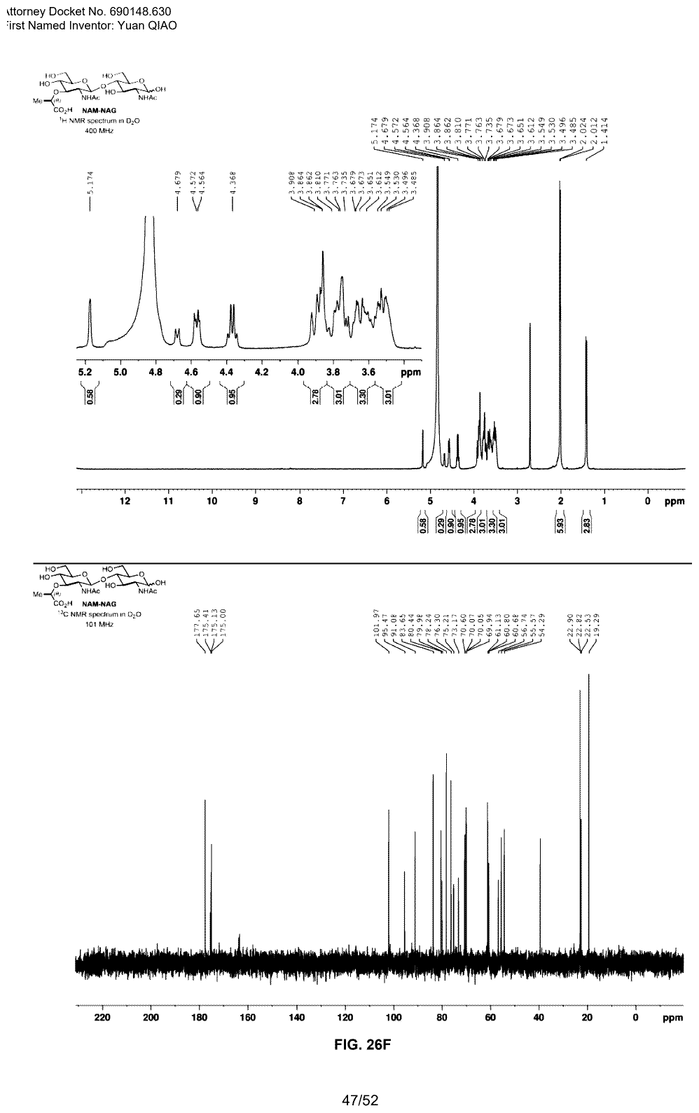

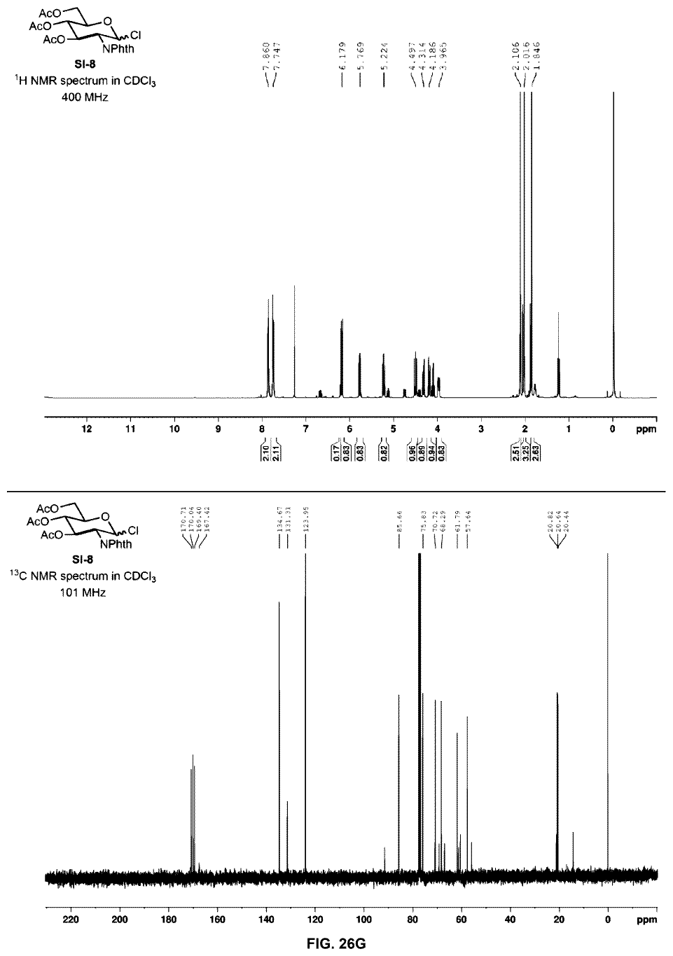

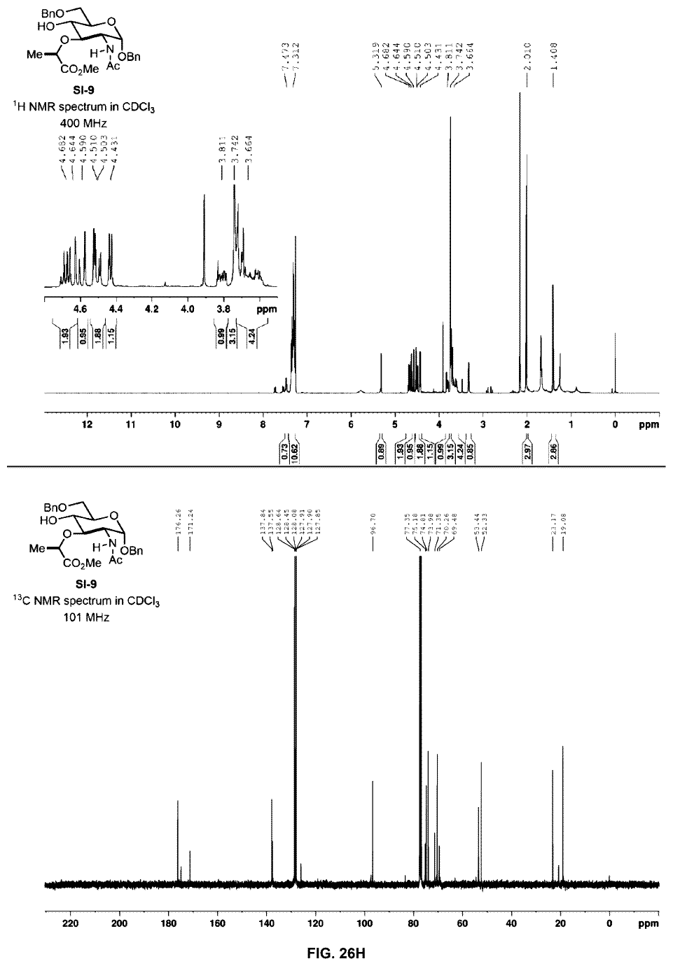

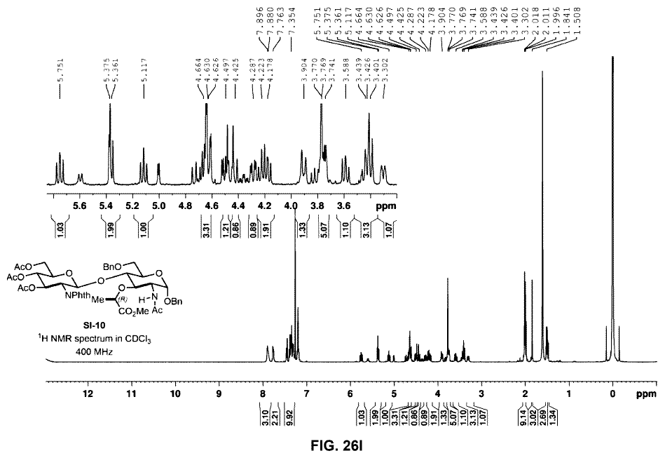

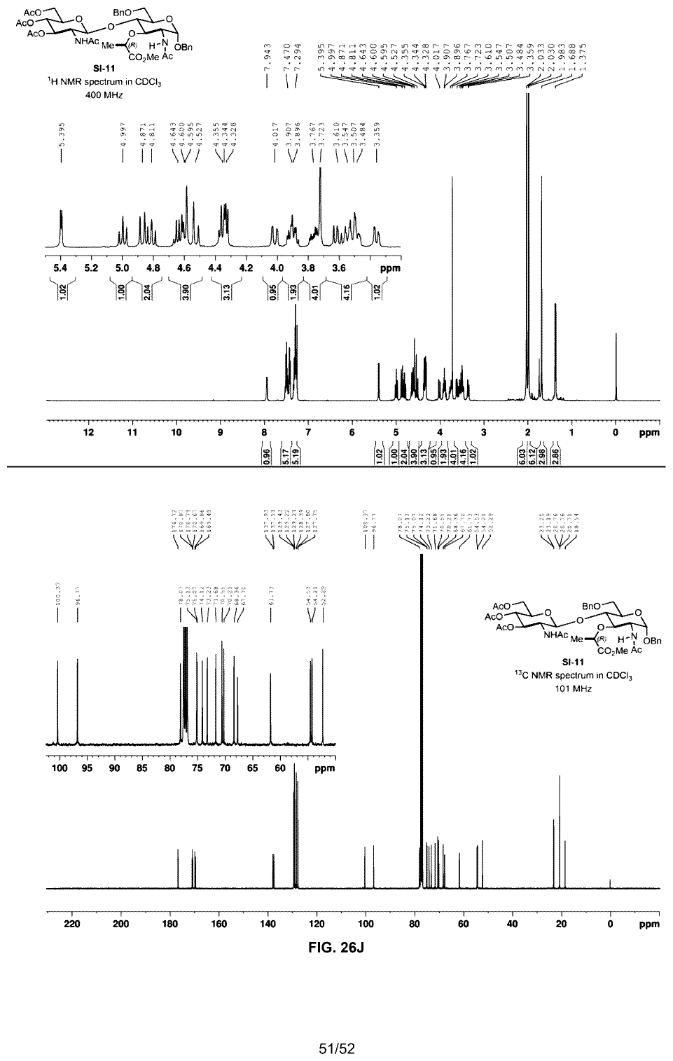

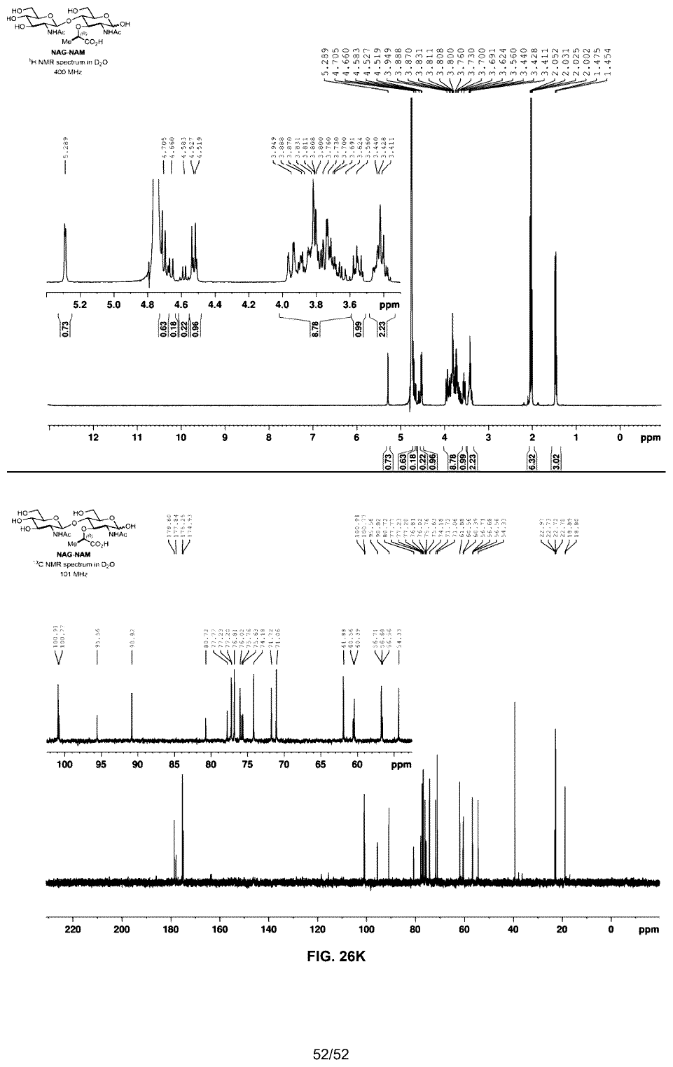

Under inert atmosphere, p-tolyl 4,6-O-benzylidene-3-O-tert-butyldimethylsilyl-2-deoxy-2-phthalimido-1-thio-β-D-glucopyranoside (3.00 g, 4.86 mmol, 1.0 eq) was stirred with 140 mL CH₂Cl₂ to obtain a clear solution. After cooling to −78° C., a solution of mCPBA (1143 mg, 5.09 mmol, 1.05 eq) in 20 mL CH₂Cl₂ was charged over 1.5 hours. The mixture was warmed to rt and concentrated to dryness. To remove m-chlorobenzoic acid, the residue was suspended in 100 mL EtOAc and washed with 4×50 mL 1% aqueous K₂CO₃. The organic extract was washed with brine, dried (Na₂SO₄), filtered, and concentrated to dryness. The crude product was purified by column chromatography (100 g silica, 10 then 20% EtOAc in PhMe) to afford sulfoxide SI-1 (3.09, 4.88 mmol, 100% yield) as a foamy semisolid. To note, the ¹H NMR spectrum indicates a ca. 54:46 mixture of epimers at the sulphur atom and that ˜0.1 eq of EtOAc is present (FIG. 26A).

¹H NMR (400 MHz, CDCl₃, 301 K, δ): 7.88 to 7.57 (m, 4H), 7.45 (m, 3H), 7.35 (m, 4H), 7.20 (m, 2H), 6.99 (d, J=8.0 Hz, 1H), 5.50 & 5.49 (s & s, 3H; PhCH(OR)₂), 5.30 & 5.21 (d J=10.3 Hz & d, J=10.2 Hz, 1H; H-1), 4.73 & 4.65 (t, J=9.9 Hz & t, J=9.9 Hz, 1H; H-2), 4.592 & 4.587 (t, J=8.8 Hz & t, J=9.1 Hz, 1H; H-3), 4.35 & 4.26 (m & dd, J=10.4, 4 Hz, 1H; H-6a), 3.83 to 3.62 (m, 2H; H-6b & H-5), 3.56 (m, 1H; H-4), 2.26 & 2.09 (s & s, 3H), 0.58 & 0.57 (s & s, 9H), −0.16 & 0.17 (s & s, 3H), −0.36 &−0.39 (s & s, 3H).

¹³C NMR (101 MHz, CDCl₃, 301 K, δ): 168.3 (br), 167.0 (br), 142.2, 141.3, 137.9, 137.0, 136.9, 136.1, 136.0, 134.4, 134.0, 131.7, 129.7, 129.5, 129.2, 129.1, 128.3, 128.2, 126.4, 125.9, 125.3, 124.6, 123.3 (br), 102.13 & 102.09 (PhCH(OR)₂), 90.5 & 88.6 (C-1), 82.0 & 81.7 (C-4), 71.1 & 70.9 (C-5), 70.86 & 70.83 (C-3), 68.2 (C-6), 52.6 & 50.7 (C-2), 25.43, 25.41, 21.4, 21.2, 17.8, 17.7, 0.1, −4.17, −4.21, −5.22, −5.25.

HRMS: the expected H+ or Na+ adducts were unable to be observed.

The CH₂Cl₂ used in this reaction was dried three times over activated MS3 Å). To a rbf was charged p-tolyl 6-O-benzyl-2-deoxy-2-phthalimido-1-thio-β-D-glucopyranoside (2.41 g, 4.76 mmol, 1.4 eq), 4-allyl-1,2-dimethoxybenzene (2.91 mL, 17.0 mmol, 5.0 eq), 2,4,6-tri-tertbutylpyrimidine (1.90 g, 7.65 mmol, 2.25 eq), and a stir bar. The mixture was co-evaporated 3× with PhMe, 8 g freshly activated MS3 Å was charged, and an inert atmosphere was secured. 100 mL CH₂Cl₂ was charged and the mixture was stirred for 1 hour. After cooling to −78° C., a solution of Tf2O (799 μL, 3.40 mmol, 1.4 eq) in 7 mL CH₂Cl₂ was charged over 15 minutes. A solution of glycosyl donor SI-1 (2.15 g, 3.40 mmol, 1.0 eq) in 20 mL CH₂Cl₂ was then charged over 2.5 hours. The mixture was warmed to −40° C. and stirred for an additional 1.5 hours. The reaction was quenched by charging 100 mL sat. aq. NaHCO₃, and the mixture was warmed to rt. After filtration through Celite, the layers were separated. The organic extract was dried over Na₂SO₄, filtered, and concentrated to dryness. The crude product was purified by column chromatography (150 g silica, 5 then 7.5% EtOAc in PhMe) to afford ˜2 g of partially purified material. This was purified again by column chromatography (120 g silica, 6.5 to 7.5% EtOAc in PhMe) to afford SI-2 (1.21 g, 1.21 mmol, 36% yield) as a semisolid (FIG. 26B). This reaction generated a 3:1 mixture of the desired product and its slightly more polar regioisomer. Assignments were made using the interring HMBC correlations.

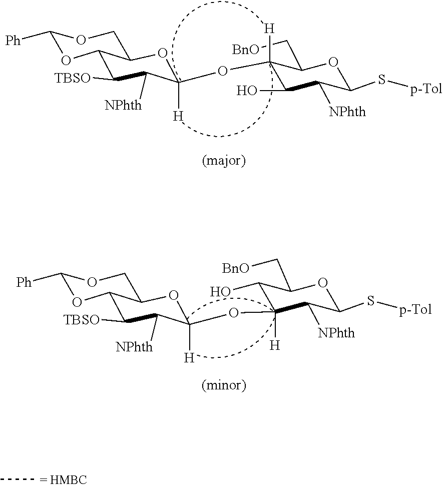

¹H NMR (400 MHz, CDCl₃, 301 K, δ): 7.90 (m, 2H), 7.83 (m 1H), 7.73 (m, 5H), 7.47 (m, 2H), 7.35 (m, 6H), 7.25 (d, J=7.7 Hz, 2H), 7.14 (d, J=7.1 Hz, 2H), 6.92 (d, J=7.7 Hz, 2H), 5.49 (s, 1H; PhCH(OR)₂), 5.45 (d, J=10.5 Hz, 1H; H-1), 5.34 (d, J=8.6 Hz, 1H; H-1′), 4.68 (dd, J=9.9, 8.9 Hz, 1H; H-3′), 4.37 (dd, J=10.2, 8.6 Hz, 1H; H-3), 4.33 (m, 1H; H-6′a), 4.27 (dd, J=10.0, 8.7 Hz, 1H; H-2′), 4.18 (t, J=10.4 Hz, 1H; H-2), 4.13 (m, 2H; PhCH₂OR), 3.72 (t, J=9.4 Hz, 1H; H-4), 3.703 (m, 1H; H-6′b), 3.696 (m, 1H; H-5′), 3.56 (t, J=8.8 Hz, 1H; H-4′), 3.52 (ddd, J=9.7, 3.8, 1.7 Hz, 1H; H-5), 3.32 (dd, J=11.4, 1.7 Hz, 1H; H-6a), 3.27 (dd, J=14, 3.8 Hz; H-6b), 2.23 (s, 3H), 0.57 (s, 9H), −0.14 (s, 3H), −0.30 (s, 3H).

¹³C NMR (101 MHz, CDCl₃, 301 K, δ): 168.3 (br), 168.2, 167.6, 167.5 (br), 138.22, 138.15, 136.8, 134.3, 134.1, 133.7, 131.9, 131.75, 131.70, 131.6, 129.5, 129.2, 128.2, 127.6, 127.45, 127.38, 126.4, 123.8, 123.6, 123.3, 102.1 (PhCH(OR)₂), 99.7 (C-1′), 83.1 (C-1), 82.1 (C-4′), 81.3 (C-4), 78.0 (C-5), 73.0 (PhCH₂OR), 71.0 (C-3), 69.3 (C-3′), 68.3 (C-6′), 68.1 (C-6), 66.2 (C-5′), 57.6 (C-2′), 55.1 (C-2), 25.4, 21.1, 17.7, −4.1, −5.4.

HRMS: the expected H+ or Na+ adducts were unable to be observed.

SI-2 (1.21 g, 1.21 mmol, 1.0 eq) was stirred with 12 mL THF, 12 mL EtOH, and 24 mL ACN. Ethylenediamine (1613 μL, 24.2 mmol, 20 eq) was charged, and the mixture was stirred in a 70-75° C. bath to obtain a clear solution. The reaction was monitored by MS and the removal of both phthalimide groups was deemed complete after 6 days. The reaction was cooled to rt and concentrated. The crude was suspended in 50 mL EtOAc and the slimy white debris was removed by filtration through Celite. The filtrate was concentrated to obtain ˜1 g of an oil. This material was stirred with 20 mL CH₂Cl₂ and 20 mL MeOH to obtain a clear solution, and then 1143 μL Ac₂O (12.1 mmol, 10.0 eq) was charged. After 1 hour, the mixture was concentrated to dryness. The residue was suspended in EtOAc and washed 2× with pH 2 buffer, washed with sat. aq. NaHCO₃, washed with brine, dried (Na₂SO₄), filtered, and concentrated. The crude material was purified by column chromatography (40 g silica, 60 to 100% v/v EtOAc in hexane) to afford SI-3 (0.53 g, 0.644 mmol, 53%) as a white solid (FIG. 26C).

¹H NMR (400 MHz, CDCl₃, 301 K, δ): 7.38 (m, 4H), 7.28 (m, 6H), 7.21 (m, 2H), 6.92 (d, J=7.7 Hz, 2H) 6.92 (br s, 1H), 6.86 (br s, 1H), 5.18 (s, 1H), 4.84 (d, J=10.1 Hz, 1H), 4.61 (d, J=8.6 Hz, 1H), 4.58 (d, J=12.0 Hz, 1H), 4.39 (d, J=12.0 Hz, 1H), 4.37 (br s, 1H), 4.24 (dd, J=10.2, 4.2 Hz, 1H), 3.86 (m, 1H), 3.84 (m, 1H), 3.83 (m, 1H), 3.79 (m, 1H), 3.78 (m, 1H), 3.66 (m, 1H), 3.62 (t, J=10.5 Hz, 1H), 3.57 (m, 1H), 3.47 (m, 1H), 3.45 (m, 1H), 3.37 (t, J=8.8 Hz, 1H), 2.19 (s, 3H), 2.07 (s, 3H), 1.90 (s, 3H), 0.81 (s, 9H), 0.02 (s, 3H), −0.04 (s, 3H).

¹³C NMR (101 MHz, CDCl₃, 301 K, δ): 171.0, 170.5, 138.68, 137.70, 137.1, 132.8, 129.7, 129.3, 129.1, 128.5, 128.13, 128.08, 127.8, 126.4, 103.0, 101.6, 86.5, 82.9, 81.6, 78.4, 74.2, 73.7, 72.5, 69.1, 68.1, 66.4, 57.0, 55.0, 25.8, 23.7, 23.6, 21.1, 18.2, −3.8, −4.7.

HRMS (ESI-TOF, m/z): calc'd for C₄₃H₅₉N₂O₁₀SSi⁺ ([M+H]⁺) 823.3654; found 823.3651.

To a rbf charged with SI-3 (100 mg, 0.121 mmol, 1.0 eq) was charged 1.2 mL ACN, 0.3 mL DMF, n-Bu₄NI (4.5 mg, 0.012 mmol, 0.1 eq), and BnBr (22 μL, 0.182 mmol, 1.5 eq). The mixture was stirred, NaH (7.3 mg, 0.182 mmol, 1.5 eq) was charged, and an inert atmosphere was secured. The mixture was heated in a 40° C. bath for 4 hours and then cooled to rt. The reaction was then quench by charging 5 mL of 20 mM aq. HCl. The resulting slurry was filtered through sintered glass, and the solids were rinsed with water and hexane. The solids were re-constituted in EtOAc and concentrated to dryness to obtain 145 mg of crude SI-4 as a semisolid. This material was dissolved stirred with 2 mL THF and 1 M TBAF in THF (0.24 mL, 0.24 mmol, 2.0 eq) was charged. After 15 hours, the mixture was diluted with EtOAc, washed twice with pH 2 buffer, washed with sat. aq. NaHCO₃, washed 4× with water, and concentrated. The solid residue was triturated twice with 5 mL Et₂O. The crude material was purified by column chromatography (20 g silica, 5 to 6% MeOH in CH₂Cl₂) to obtain SI-5 (65 mg, 81.3 μmol, 67% yield) as a white solid (FIG. 26D).

¹H NMR (400 MHz, DMSO-d₆, 301 K, δ): 8.06 (d, J=9.1 Hz, 1H), 7.97 (d, J=8.6 Hz, 1H), 7.34 (m, 17H), 7.03 (d, J=7.1 Hz, 2H), 5.55 (s, 1H; PhCH(OR)₂), 5.32 (d, J=5.3 Hz, 1H; ROH), 4.81 (d, J=11.1 Hz, 1H; PhCH₂OR at C-3), 4.78 (d, J=11.1 Hz, 1H; H-1), 4.62 (d, J=8.1 Hz, 1H; H-1′), 4.55 (d, J=11.1 Hz, 1H; PhCH₂OR at C-3), 4.54 (s, 2H; PhCH₂OR at C-6), 3.97 (dd, J=10.0, 4.8 Hz, 1H; H-6′a), 3.80 (m, 1H, H-2), 3.79 (d, J=11.1 Hz, 1H; H-6a), 3.74 (d, J=9.1 Hz, 1H; H-4), 3.65 (dd, J=11.1, 5.2 Hz, 1H; H-6b), 3.59 (m, 1H; H-3′), 3.57 (m, 1H; H-3), 3.55 (m, 1H; H-2′), 3.53 (m, 1H; H-5), 3.48 (d, J=10.0 Hz, 1H; H-6′b), 3.40 (t, J=9.1 Hz, 1H; H-4′), 3.08 (td, J=9.5, 4.8 Hz, 1H; H-5′), 2.23 (s, 3H), 1.84 (s, 3H), 1.83 (s, 3H).

¹³C NMR (101 MHz, DMSO-d₆, 301 K, δ): 169.4, 169.0, 139.0, 138.7, 137.7, 136.3, 130.6, 130.4, 129.5, 128.9, 128.7, 128.2, 128.0, 127.4, 127.3, 127.2, 127.1, 126.4, 101.1 (C-1′), 100.6 (PhCH(OR)₂), 85.6 (C-1), 81.7 (C-3), 81.2 (C-4′), 78.3 (C-5), 75.8 (C-4), 73.4 (PhCH₂OR at C-3), 71.9 (PhCH₂OR at C-6), 70.2 (C-3), 68.8 (C-6), 67.8 (C-6′), 65.9 (C-5′), 56.9 (C-2′), 52.6 (C-2), 23.1, 22.9, 20.6.

HMBC correlations confirmed the installation of the benzyl ether at the correct position.

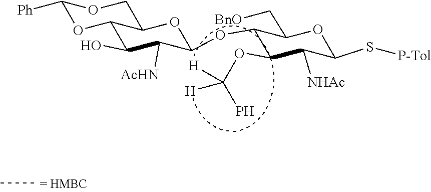

HRMS (ESI-TOF, m/z): calc'd for C₄₄H₅₁N₂O₁₀S⁺ ([M+H]⁺) 799.3259; found 799.3258.

To a stirred suspension of SI-5 (65 mg, 81.3 μmol, 1.0 eq) in 2.5 mL DMF under inert atmosphere was charged (S)-2-bromopropionic acid (73 μL, 813 μmol, 10.0 eq). The mixture was cooled in an ice bath, NaH (81 mg, 2.03 mmol, 25 eq) was charged, and the cooling bath was removed. After 4 hours, the reaction was quenched by charging AcOH (116 μL, 2.03 mmol, 25 eq). After aging 15 hours, the mixture was concentrated to dryness. The solid residue was triturated with copious water, triturated with copious 1:1 v/v Et₂O: hexane, and then dried under vacuum at 50° C. to afford SI-6 (97 mg, >100% nominal yield) as a pale tan solid. The NMR spectra indicated presence of a nonpolar impurity (FIG. 26E).

¹H NMR (400 MHz, DMSO-d₆, 301 K, δ): 8.08 (m, 2H), 7.34 (m, 17H), 7.03 (d, J=7.5 Hz, 2H), 5.63 (s, 1H), 4.80 (d, J=11.1 Hz, 1H), 4.78 (d, J=10.1 Hz), 4.68 (d, J=8.0 Hz), 4.55 (d, J=11.1 Hz, 1H), 4.53 (s, 2H), 4.19 (dd, J=11.4, 4.9 Hz, 1H), 3.97 (m, 1H), 3.84 to 3.68 (m, 3H), 3.67 to 3.53 (m, 5H), 3.11 (m, 1H), 2.23 (s, 3H), 1.83 (s, 3H), 1.82 (s, 3H), 1.23 (d, J=6.4 Hz, 3H).

¹³C NMR (101 MHz, DMSO-d₆, 301 K, δ): 174.3, 169.8, 169.0, 139.0, 138.6, 137.6, 136.3, 130.5, 130.4, 129.5, 128.8, 128.2, 128.1, 128.0, 127.35, 127.25, 127.2, 127.1, 125.8, 101.0, 100.0, 85.6, 81.7, 81.4, 78.2, 77.6, 75.9, 75.5, 73.4, 71.9, 68.7, 67.7, 65.4, 55.4, 53.1, 23.1, 22.9, 20.6, 19.0.

HRMS (ESI-TOF, m/z): calc'd for C₄₇H₅₅N₂O₁₂S⁺ ([M+H]⁺) 871.3470; found 871.3461.

To a stirred suspension of crude SI-6 (97 mg, 81.3 μmol assuming 100% yield in previous step, 1.0 eq) in 1.2 mL ACN and 0.4 mL H₂O was charged Hg (OAc) 2 (39 mg, 122 μmol, 1.5 eq). After 14 hours, the thick slurry was diluted with 3 mL ACN and 1 mL H₂O. 500 mg Lewatit TP-214 resin was then charged, and the suspension was stirred vigorously. After 1 hour, the mixture was filtered through cotton, concentrated to dryness, and triturated 3× with 3 mL 1:1 v/v Et₂O: hexane. The crude material was purified by column chromatography (12 g silica, 8:92:1 to 10:90:1 v/v MeOH:CH₂Cl₂:AcOH) to afford SI-7 (26 mg, 34.0 μmol, 42% over the previous two steps) as a white solid.

HRMS (ESI-TOF, m/z): calc'd for C₄₀H₄₉N₂O₁₃⁺ ([M+H]⁺) 765.3229; found 765.3227.

### NAM-NAG

To a stirred suspension of SI-7 (26 mg, 34.0 μmol, 1.0 eq) in 1.5 mL of 3:2:2:1 v/v i-PrOH: CHCl₃:AcOH:H₂O was added 20 mg Pd/C and 20 mg Pd(OH)₂/C. With vigorous stirring, the mixture was exposed to 1 atmosphere hydrogen via balloon. After 5 hours, the mixture was filtered through Celite, rinsed forward with MeOH, and concentrated. The residue was suspended in 2 mL H₂O and washed twice with 1 mL Et₂O. The aqueous extract was the concentrated to dryness. The crude material was purified by preparative HPLC (0 to 20% ACN in 0.1% aq. TFA) to afford NAM-NAG (11.6 mg, 23.4 μmol, 69% yield) as a white solid (FIG. 26F).

¹H NMR (400 MHz, D2O, 301 K, δ): 5.17 (d, J=2.5 Hz, 1H; H-1α), 4.68 (d, J=7.7 Hz, 1H; H-1β), 4.57 (d, J=8.4 Hz, 1H; H-1′α), 4.56 (d, J=8.3 Hz, 1H; H-1′β), 4.37 (q, J=6.8 Hz, 1H; lactoyl CHCH₃), 3.91 (d, J=12.4 Hz, 1H; H-6′α), 3.864 (m, 2H; H-3α & H-5α), 3.862 (m, 1H; H-2α), 3.81 (dd, J=12.2, 1.6 Hz, 1H; H-6aβ), 3.77 (m, 1H; H-2′), 3.76 (m, 1H; H-6aα), 3.74 (dd, J=12.4, 5.3 Hz, 1H; H-6′b), 3.68 (m, 1H; H-2β), 3.67 (m, 1H; H-3β), 3.65 (m, 1H; H-6b), 3.62 (m, 1H; H-4), 3.55 (m, 1H; H-3′), 3.53 (m, 1H; H-4′), 3.49 (m, 1H; H-5′), 3.48 (m, 1H; H-5β), 2.02 (s, 3H; acetamide), 2.01 (s, 3H; acetamide), 1.41 (d, J=6.8 Hz, 3H; lactoyl CHCH₃).

¹³C NMR (101 MHz, D₂O, 301 K, δ): 177.6, 175.4, 175.1, 175.0, 102.0 (C-1′), 95.5 (C-1β), 91.1 (C-1α), 83.6 (C-3′), 80.4 (C-4α), 80.0 (C-4β), 78.2 (lactoyl CHCH₃), 76.3 (C-5′), 75.2 (C-5β), 73.2 (C-3β), 70.6 & 69.9 (C-3α & C-5α), 70.07 (C-4′α), 70.05 (C-4′β), 61.1 (C-6′), 60.8 (C-6β), 60.7 (C-6α), 56.7 (C-2β), 55.6 (C-2′), 54.3 (C-2α), 22.9 (acetamide) 22.8 & 22.5 (acetamide), 19.3 (lactoylCHCH₃).

H-3α and H-5α have very close chemical shifts, and C-3α and C-5α have very close chemical shifts. It was not possible to distinguish these two positions.

HRMS (ESI-TOF, m/z): calc'd for C₁₉H₃₃N₂O₁₃⁺ ([M+H]⁺) 497.1977; found 497.1969.

### NAG-NAM

NAG-NAM was obtained by scaleup of a literature procedure. NMR data matched literature.

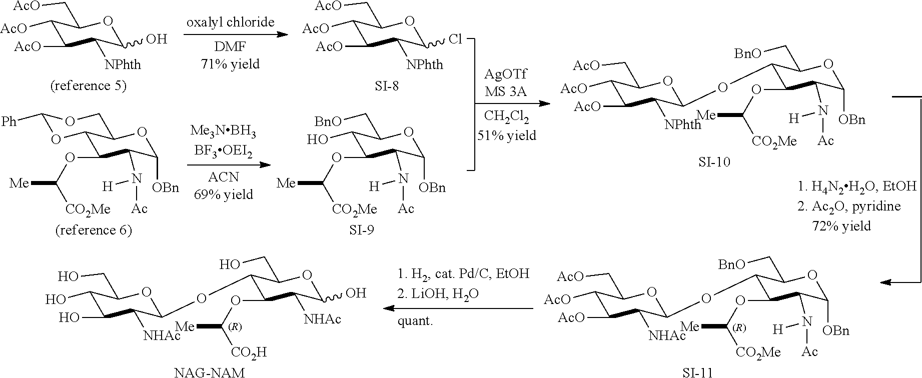

To a two-necked reaction vessel equipped with a stir bar and drying tube was charged 3,4,6-tri-O-acetyl-2-deoxy-2-phthalimido-D-glucopyranose (16.6 g, 38.0 mmol, 1.0 eq) and 98 mL DMF. The mixture was stirred to obtain a clear solution and then cooled to 0-5° C. With extreme caution, oxalyl chloride (16.6 g, 114 mmol, 3.0 eq) was charged over 30 minutes by syringe. The large quantity of gasses given off combined with the tendency of outlets to become clogged presents a safety hazard; an oversized reaction vessel is recommended). After aging for 5 hours at 0-5° C., the mixture was poured into 300 mL saturated aqueous NaHCO₃. The resulting slurry was filtered, and the solids were rinsed with water and hexane. The solids were then partly dried under vacuum to afford ca. 32 g of material. The crude product was purified by column chromatography (100 g silica, 30 then 40% EtOAc in hexane) to afford glycosyl chloride SI-8 (12.29 g, 27.0 mmol, 71% yield) as a white solid (FIG. 26G).

¹H NMR (400 MHz, CDCl₃, 301 K, δ): 7.86 (m, 2H), 7.75 (m, 2H), 6.18 (d, J=9.4 Hz, 1H), 5.77 (dd, J=10.4, 9.3 Hz, 1H), 5.22 (dd, J=10.2, 9.3 Hz, 1H), 4.50 (dd, J=10.4, 9.4 Hz, 1H), 4.31 (dd, J=12.5, 4.7 Hz, 1H), 4.19 (dd, J=12.5, 2.1 Hz, 1H), 3.96 (ddd, J=10.2, 4.7, 2.1 Hz, 1H), 2.11 (s, 3H), 2.02 (s, 3H), 1.85 (s, 3H).

¹³C NMR (101 MHz, CDCl₃, 301 K, δ): 170.7, 170.0, 169.4, 167.4 (br), 134.7, 131.3, 124.0, 85.7, 75.8, 70.7, 68.3, 61.8, 57.6, 20.8, 20.6, 20.4.

The product was obtained as a 83:17/β:α mixture; only the peaks of the major isomer are tabulated.

HRMS (ESI-TOF, m/z): the expected proton or sodium adduct were unable to be observed.

Benzyl 2-acetamido-4,6-O-benzylidene-2-deoxy-3-O—[(R)-1-(methoxycarbonyl)ethyl]-α-Dglucopyranside (6.59 g, 13.6 mmol, 1.0 eq) was stirred with 120 mL ACN to afford a thick white slurry. BH₃·NMe₃ (1188 mg, 16.3 mmol, 1.2 eq) was charged, and the mixture was cooled to 0-5° C. BF₃·OEt₂ (10.5 mL, 84.8 mmol, 6.25 eq) was charged over 5 minutes, after which the reaction became a clear solution. After aging for 2 hours at 0-5° C., 100 mL saturated aqueous NaHCO₃ was charged and the mixture was stirred vigorously. After 30 minutes, 200 mL EtOAc was charged, the mixture was extracted, the layers were separated, and the organic layer was stirred with 100 mL 1 M aqueous HCl. After 1 hour, the layers were separated, and the organic layer was washed with 50 mL 1 M aqueous Na₂CO₃ (×4) and brine. The organic extract was dried (Na₂SO₄), filtered, and concentrated. The crude material (8.6 g) was purified by column chromatography (200 g silica, 50 then 70% EtOAc in hexane) to afford glycosyl acceptor SI-9 (4.58 g, 9.39 mmol, 69% yield). Particular attention should be given to the aqueous workup because boron-containing impurities are not easily purged by column chromatography (FIG. 26H).

¹H NMR (400 MHz, CDCl₃, 301 K, δ): 7.46 (d, J=5.8 Hz, 1H), 7.32 (m, 10H), 5.32 (d, J=3.4 Hz, 1H), 4.68 (d, J=7.0 Hz, 1H), 4.65 (d, J=12.2 Hz, 1H), 4.59 (d, J=11.8 Hz, 1H), 4.51 (d, J=11.8 Hz, 1H), 4.51 (d, J=12.2 Hz, 1H), 3.82 (ddd J=10.7, 5.1, 3.5 Hz, 1H), 3.74 (s, 3H), 3.730 (m, 1H), 3.729 (m, 1H), 3.70 (m, 1H), 3.66 (m, 1H), 3.61 (m, 1H), 2.01 (s, 3H), 1.41 (s, J=7.0 Hz, 3H).

¹³C NMR (101 MHz, CDCl₃, 301 K, δ): 176.3, 171.2, 137.8, 137.6, 128.6, 128.5, 128.1, 127.91, 127.90, 127.85, 96.7, 77.4, 75.2, 74.8, 74.0, 71.4, 70.3, 69.5, 53.4, 52.3, 23.2, 19.1.

HRMS (ESI-TOF, m/z): calc'd for C₂₆H₃₄NO₈⁺ ([M+H]⁺) 488.2279; found 488.2272.

To an oven-dried 2-necked rbf equipped with a stirbar, argon inlet, and glass stopper was charged AgOTf (3.66 g, 14.3 mmol, 4.0 eq). The solids were stirred under vacuum in a 90° C. bath for 2 hours and then cooled to room temperature. The reactor was backfilled with argon, 4 g of activated MS3 Å was charged, and the glass stopper was replaced with a rubber septum. A solution of glycosyl acceptor SI-9 (1.73 g, 3.56 mmol, 1.0 eq) in 15 mL CH₂Cl₂ was charged by syringe, and the mixture was stirred for 1 hour. A solution of 4.86 g glycosyl donor SI-8 (10.7 mmol, 3.0 eq) was then charged over 1 hour by syringe. After aging 1 hour, the mixture was then diluted with 150 mL EtOAc and extracted with saturated aqueous NaHCO₃. The organic material was washed with brine, dried (Na₂SO₄), filtered, and concentrated. The crude material was purified by column chromatography (150 g silica, 64:32:5 then 64:32:7 v/v Et₂O:hexane:i-PrOH). The material thus obtained was stirred with 10 mL Et₂O to obtain a white slurry. 5 mL hexane was charged, and the mixture was cooled to 0-5° C. The slurry was filtered, and the solids were washed with hexane and dried to afford disaccharide SI-10 (1.63 g, 1.80 mmol, 51% yield) as a white solid (FIG. 26I).

¹H NMR (400 MHz, CDCl₃, 301 K, δ): 7.90 (m, 1H), 7.88 (m, 2H), 7.76 (m, 2H), 7.35 (m, 10H), 5.75 (t, J=9.8 Hz, 1H), 5.37 (d, J=3.2 Hz, 1H), 5.36 (d, J=8.3 Hz, 1H), 5.12 (t, J=9.6 Hz, 1H), 4.66 (q, J=6.9 Hz, 1H), 4.630 (d, J=12.5 Hz, 1H), 4.626 (d, J=12.5 Hz, 1H), 4.50 (d, J=12.1 Hz, 1H), 4.43 (d, J=12.1 Hz, 1H), 4.29 (dd, J=12.5, 3.7 Hz, 1H), 4.22 (t, J=9.2 Hz, 1H), 4.18 (dd, J=10.4, 8.5 Hz, 1H), 3.90 (dd, J=12.4, 1.8 Hz, 1H), 3.770 (s, 3H), 3.769 (m, 1H), 3.74 (m, 1H), 3.59 (dd, J=10.7, 9.1 Hz, 1H), 3.44 (m, 1H), 3.43 (m, 1H), 3.40 (m, 1H), 3.30 (dt, J=9.9, 2.4 Hz, 1H), 2.02 (s, 3H), 2.01 (s, 3H), 2.00 (s, 3H), 1.84 (s, 3H), 1.51 (d, J=6.9 Hz, 3H).

HRMS (ESI-TOF, m/z): calc'd for C₄₆H₅₃N₂O₁₇⁺ ([M+H]⁺) 905.3339; found 905.3323.

1. Particular attention should be given to the water content of the solvent. dichloromethane dried 3 times over activated MS3 Å was used.
2. The reaction is not easy to monitor by TLC. Full consumption of the glycosyl acceptor is indicated by a purple/black endpoint. Completion can be confirmed by withdrawing a few μL of reaction mixture by syringe and analysing by MS. If moisture is properly excluded from the reaction, then an endpoint will be reached well before 3.0 eq of the glycosyl acceptor has been added.
3. A muramic acid-derived impurity was present in the product at a ca. 45 mol % level. This issue did not occur in the reported synthetic route, and the reason for the discrepancy is unknown. The impurity was easily purged at the next step.

SI-10 (3.05 g, 3.37 mmol, 1.0 eq) was stirred with 150 mL EtOH and 3 mL H₂O. Hydrazine hydrate (1147 μL, 23.6 mmol, 7.0 eq) was charged. The mixture was heated in a 36° C. bath. After 41 hours, an additional 164 μL hydrazine hydrate (3.37 mmol, 1.0 eq) was charged. After 7 more hours, the mixture was cooled to room temperature. 2.5 mL HOAc was added, and the mixture was concentrated and co-evaporated with pyridine (×3). The residue was suspended in 50 mL pyridine and 25 mL Ac₂O was charged. After 13 hours of stirring, the mixture was concentrated and co-evaporated with PhMe (×3). The residue was taken up in EtOAc, washed with pH 2 buffer (×2) washed with saturated aqueous NaHCO3 (×2), and washed with brine. The organic extract was dried (Na₂SO₄), filtered, and concentrated. The crude material was purified by column chromatography (100 g silica, 5:5:1 v/v hexane:EtOAc:i-PrOH). The material thus obtained was triturated with 3 mL Et₂O (×4) to afford SI-11 (1.98 g, 2.42 mmol, 72% yield) as a white solid (FIG. 26J).

¹H NMR (400 MHz, CDCl₃, 301 K, δ): 7.94 (d, J=4.3 Hz, 1H), 7.47 (m, 5H), 7.29 (m, 5H), 5.40 (d, J=3.3 Hz, 1H), 5.00 (t, J=9.7 Hz, 1H), 4.87 (d, J=11.9 Hz, 1H), 4.81 (t, J=9.8 Hz, 1H), 4.64 (q, J=7.0 Hz, 1H), 4.60 (d, J=12.3 Hz, 1H), 4.59 (d, J=8.0 Hz, 1H), 4.53 (d, J=12.3 Hz, 1H), 4.35 (dd, J=12.4, 4.9 Hz, 1H), 4.34 (d, J=11.9 Hz, 1H), 4.33 (d, J=8.6 Hz, 1H), 4.02 (dd, J=12.4, 1.7 Hz, 1H), 3.91 (m, 1H), 3.90 (m, 1H), 3.77 (m, 1H), 3.72 (s, 3H), 3.61 (dd, J=10.9, 9.0 Hz, 1H), 3.55 (m, 1H), 3.51 (m, 1H), 3.48 (m, 1H), 3.36 (dd, J=10.6, 2.0 Hz, 1H), 2.034 (s, 3H), 2.030 (s, 3H), 1.98 (two s, 6H), 1.69 (s, 3H), 1.38 (d, J=7.0 Hz, 3H).

¹³C NMR (101 MHz, CDCl₃, 301 K, δ): 176.7, 170.9, 170.8, 170.7, 169.9, 169.5, 137.9, 137.5, 129.4, 129.22, 129.21, 128.4, 127.8, 127.7, 100.4, 96.8, 78.1, 75.13, 75.07, 74.1, 73.2, 71.7, 70.6, 70.2, 68.4, 67.7, 61.7, 54.5, 54.2, 52.3, 23.20, 23.19, 20.76, 20.76, 20.71, 18.5.

HRMS (ESI-TOF, m/z): calc'd for C₄₀H₅₃N₂O₁₆⁺ ([M+H]⁺) 817.3390; found 817.3378.

SI-11 (977 mg, 1.20 mmol, 1.0 eq) was stirred with 24 mL EtOH. 195 mg Pd/C was charged, and the mixture was stirred vigorously under H₂ (balloon). After 16 hours, the mixture was filtered through Celite and rinsed forward with MeOH. The filtrate was concentrated to dryness and then stirred with 6 mL H₂O. The clear solution thus obtained was cooled to 0-5° C. and 0.5 M aqueous LiOH (6.0 mmol, 5.0 eq) was charged. After 1.5 hours, the pH was adjusted to ca. 3 using Amberlite IR-120 H⁺. The mixture was filtered through cotton, rinsed forward with H₂O, and the filtrate was concentrated. The residue was purified on 10 g Sephadex G-25, eluting with 0.1% aqueous TFA. The fractions containing the product were concentrated, taken up in 10 mL H₂O, and washed with 10 mL CH₂Cl₂. The aqueous extract was lyophilized to obtain NAG-NAM (595 mg, 1.20 mmol, quantitative yield) as a white solid (FIG. 26K).

¹H NMR (400 MHz, D₂O, 301 K, δ): 5.29 (d, J=2.6 Hz, 1H; H-1α), 4.70 (d, J=7.0 Hz, 1H; H-1β), 4.66 (q, J=6.9 Hz, 1H; lactoyl CHCH₃α), 4.58 (q, J=6.8 Hz, 1H; lactoyl CHCH₃β), 4.53 (d, J=8.2 Hz, 1H; C-1′α), 4.52 (d, J=8.1 Hz, 1H; C-1′β), 3.95 (d, J=12.6 Hz, 1H; H-6′α), 3.89 (m, 1H;H-4), 3.87 (m, 1H; H-6aβ), 3.83 (m, 1H; H-5α), 3.811 (m, 1H; H-2α), 3.808 (m, 1H; H-3α), 3.80 (m, 1H; H-6aα), 3.76 (m, 1H; H-6′b), 3.73 (m, 1H; H-2′), 3.70 (m, 1H; H-6b), 3.69 (m, 1H; H-2β), 3.62 (t, J=9.5 Hz, 1H; H-3β), 3.56 (t, J=9.5 Hz, 1H; H-3′), 3.44 (m, 1H; H-5β), 3.43 (m, 1H; H-5′), 3.41 (m, 1H; H-4′), 2.05, 2.032, 2.025 & 2.00 (acetamide), 1.48 (d, J=6.9 Hz, 3H; lactoyl CHCH₃α), 1.45 (d, J=6.8 Hz, 3H; lactoyl CHCH₃β).

¹³C NMR (101 MHz, D₂O, 301 K, δ): 178.6, 177.8, 175.3, 174.9, 100.91 (C-1′α), 100.77 (C-1′β), 95.6 (C-1β), 90.8 (C-1α), 80.7 (C-3β), 77.8 (lactoyl CHCH₃β), 77.23 (lactoyl CHCH₃α), 77.20 (C-3α), 76.8 (C-5′), 76.0 (C-4α), 75.8 (C-5β), 75.6 (C-4β), 74.2 (C-3′), 71.7 (C-5α), 71.1 (C-4′), 61.9 (C-6′), 60.6 (C-6β) 60.4 (C-6α), 56.71 (C-2′α), 56.68 (C-2′β), 56.56 (C-2β), 54.3 (C-2α), 23.0, 22.73, 22.72 & 22.70 (acetamide), 18.9 (lactoyl CHCH₃β), 18.8 (lactoyl CHCH₃α).

HRMS (ESI-TOF, m/z): calc'd for C₁₉H₃₃N₂O₁₃⁺ ([M+H]⁺) 497.1977; found 497.1981.

### Results

### Example 1: Muramic Acid (MurN) Analysis Affords Global Quantification of Soluble PGNs in Hosts

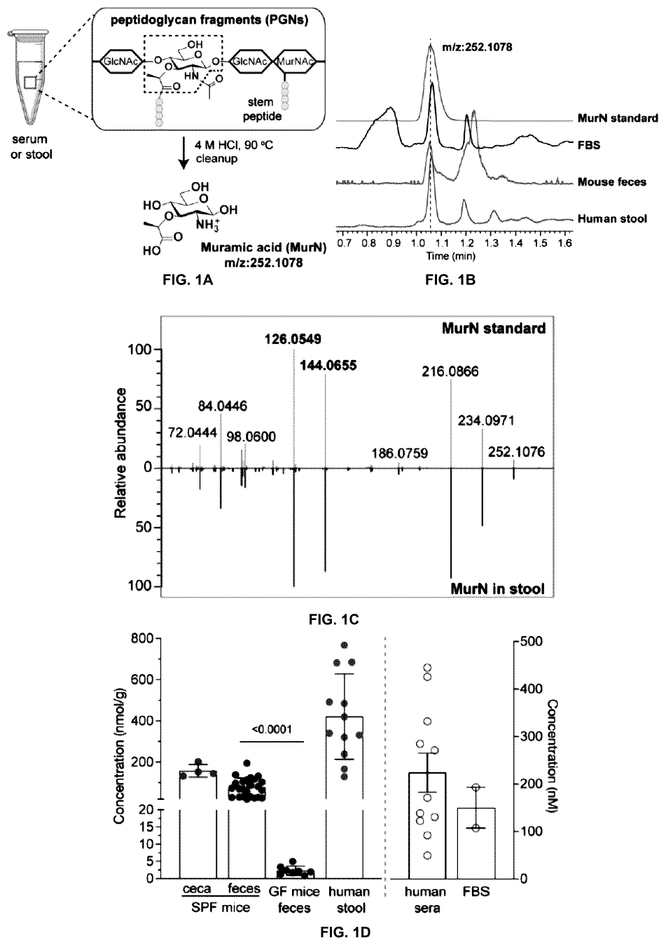

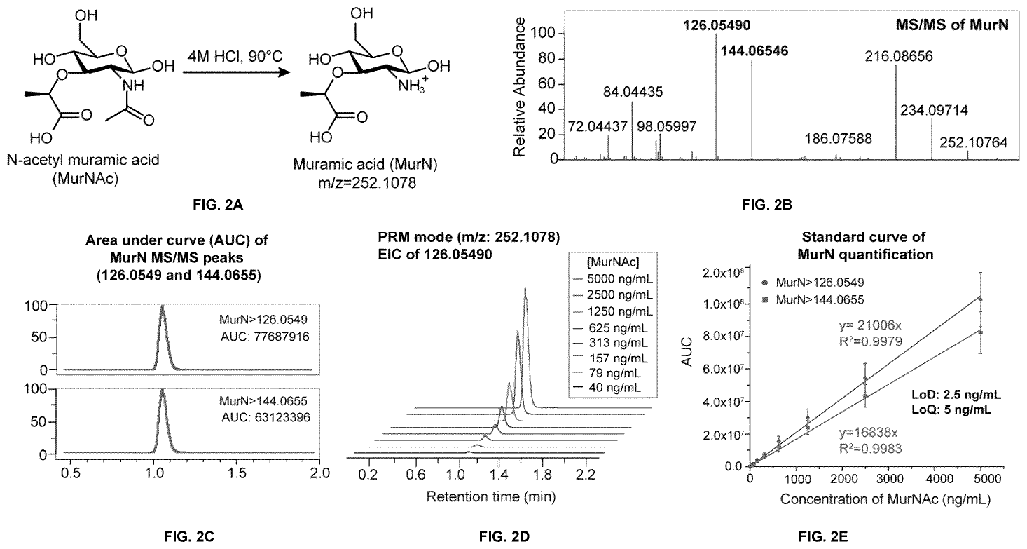

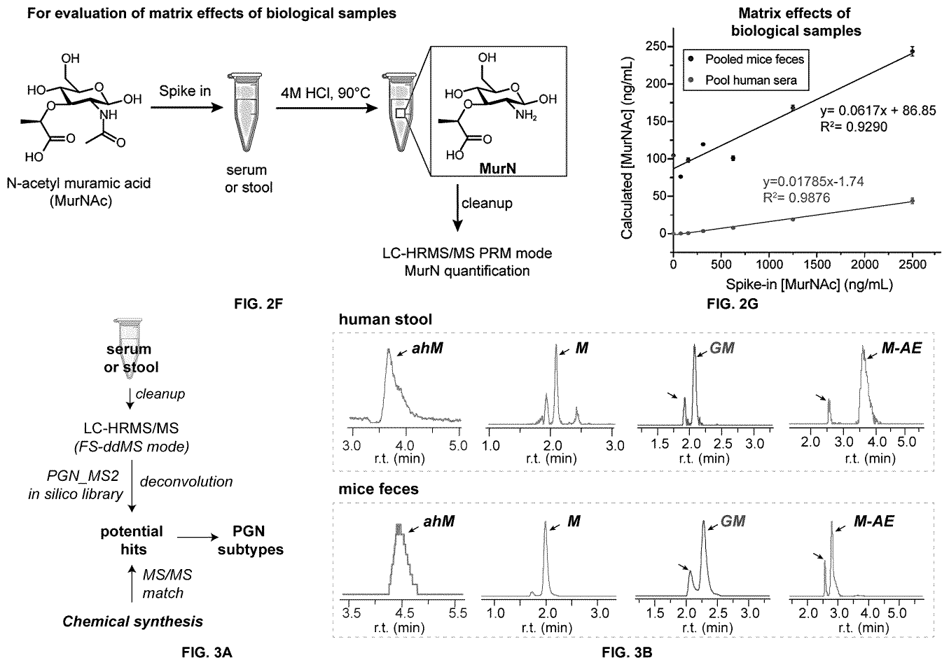

Acid hydrolysis of PGNs readily releases muramic acid (MurN), an invariant constituent of bacterial peptidoglycan that is absent in mammalian metabolites, rendering it an ideal unit for PGN quantification. Upon proper sample cleanup, MurN (m/z: 252.1078) was detected in both sera and feces of mice and humans (FIG. 1A-C), supporting the ubiquitous existence of gut microbiota-derived PGNs in hosts. Quantification of MurN was achieved based on the abundance of its two prominent daughter ions (m/z: 126.0549 and 144.0655) using the parallel reaction monitoring (PRM) mode of LC-HRMS/MS (FIG. 1C). Calibration curves were established with serial dilutions of N-acetyl-muramic acid (MurNAc) in water and spiked pooled sera or feces (FIG. 2A-2G). Next, endogenous MurN was quantified in different biological samples with adjustments for the respective matrix effects. Ceca and feces from specific pathogen-free (SPF) mice contain a considerable amount of MurN, while feces from germ-free (GF) mice show virtually undetectable levels (FIG. 1D). Healthy human stools manifest higher MurN abundance but with considerable individual variability. Healthy human sera also display a wide range of MurN concentrations, averaging approximately 200 nM, which is comparable to the amount of MurN in two brands of fetal bovine serum (FBS). Notably, previous icELISA studies also revealed similar PGN concentrations in human sera,[15] supporting the robustness and sensitivity of the quantification workflow. Together, the LCHRMS/MS-based MurN analysis provides a quantitative perspective of gut microbiota-derived soluble PGNs in hosts.

### Example 2: Saccharide Moieties Represent Predominant PGN Subtypes in the Host Gut

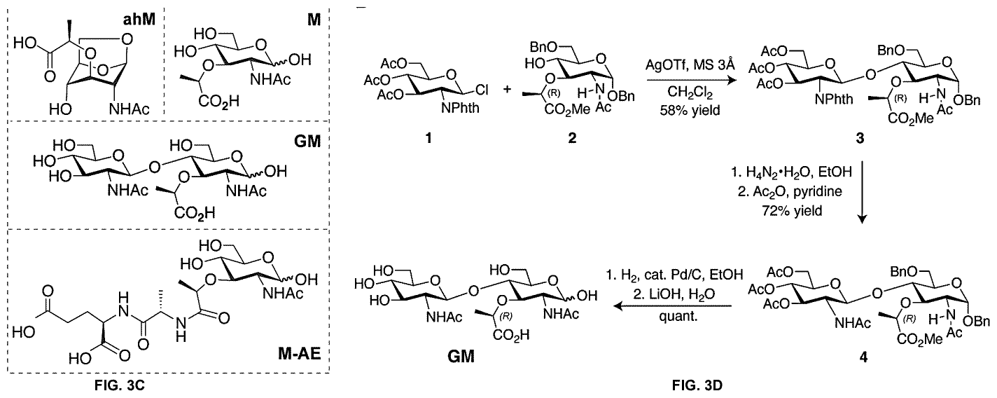

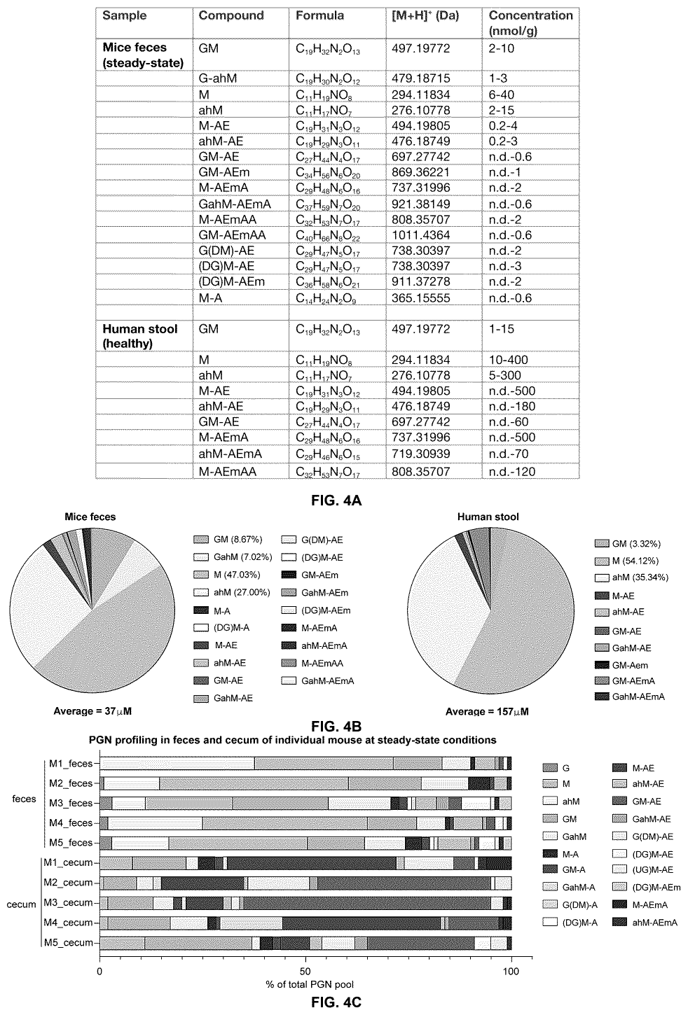

For structural profiling of gut microbiota-derived PGNs, the cleaned-up sera or stool samples were directly subjected to an untargeted analysis using the full-scan and data-dependent analysis (FSddA) mode of LC-MS/MS. Facilitated by recently developed in silico PGN_MS2 spectral library,[13] the experimental LC-MS/MS data for PGN identification was extensively evaluated, where hits were prioritized according to rigorous matching criteria including MS1 accuracy, isotopic pattern, and MS/MS fragmentation pattern (FIG. 3A). To validate potential PGN hits, a panel of PGNs was chemically synthesized as authentic standards (FIG. 3B-3D). With this workflow, the diversity and abundance of PGNs were found to be significantly higher in the human stool than in sera, which is consistent with the rapid excretion and overall biodistribution of gut microbiota-derived PGNs in hosts. [19] Interestingly, the major PGN subtypes in human stools and mouse feces are highly similar (FIG. 4A-4C), suggesting that closely related gut bacteria may be the primary contributors of soluble PGNs, or that both mammalian hosts encode similar enzymes for processing PGNs.

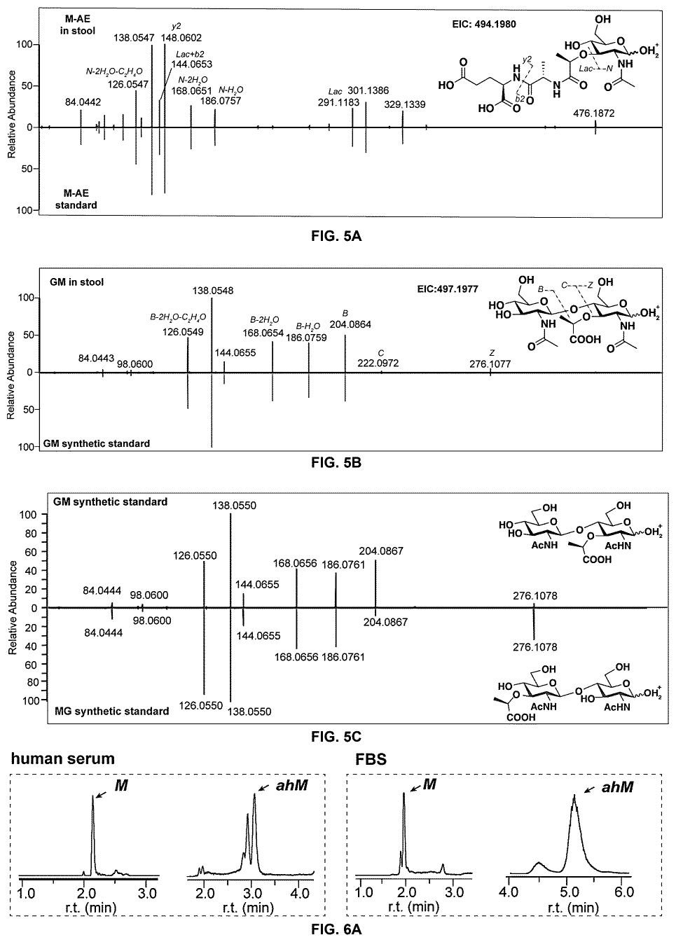

The soluble PGNs in the host gut can be classified into two groups: saccharide moieties that lack any stem peptide, and typical muropeptides containing both glycan backbone and stem peptide (FIG. 3B-3C, FIG. 4A-4C). The PGN-derived saccharide moieties, including MurNAc (M), 1,6-anhydro-MurNAc (ahM), GlcNAc-MurNAc (GM), and GlcNAc-ahMurNAc (GahM) are ubiquitously present in every sample of the cohort. In contrast, variable forms of muropeptides are detected across individuals, yet they mostly belong to the iE-DAPcontaining PGNs. In addition, the abundance of muramyl-dipeptides GM-AE and M-AE was revealed in mice ceca, which are close analogues of the widely used MDP (i.e. M-AQ) but differ in the amidation state of the stem peptide (FIG. 5A-5C). Furthermore, muropeptides with diacetylated GlcNAc or MurNAc moieties were detected, which represent peptidoglycan modifications that confer lysozyme resistance in many gut bacteria. [20] LC-MS quantification of these major PGN subtypes showed that the saccharide moieties account for nearly 90% of all soluble PGNs in host feces. The diverse spectrum of natural PGNs in the host gut underscores the need to explore PGN bioactivities beyond the well-studied model ligands.

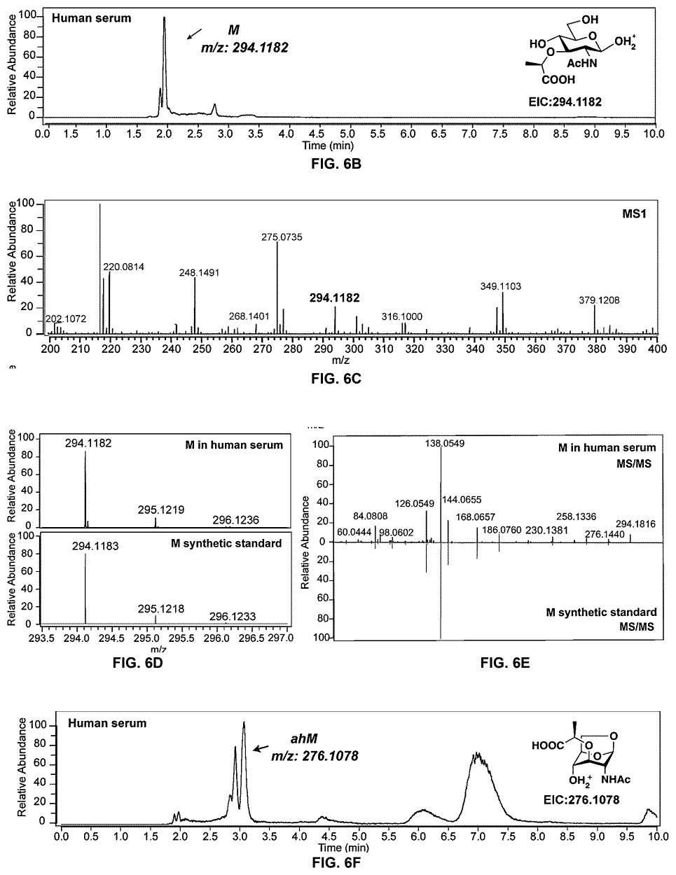

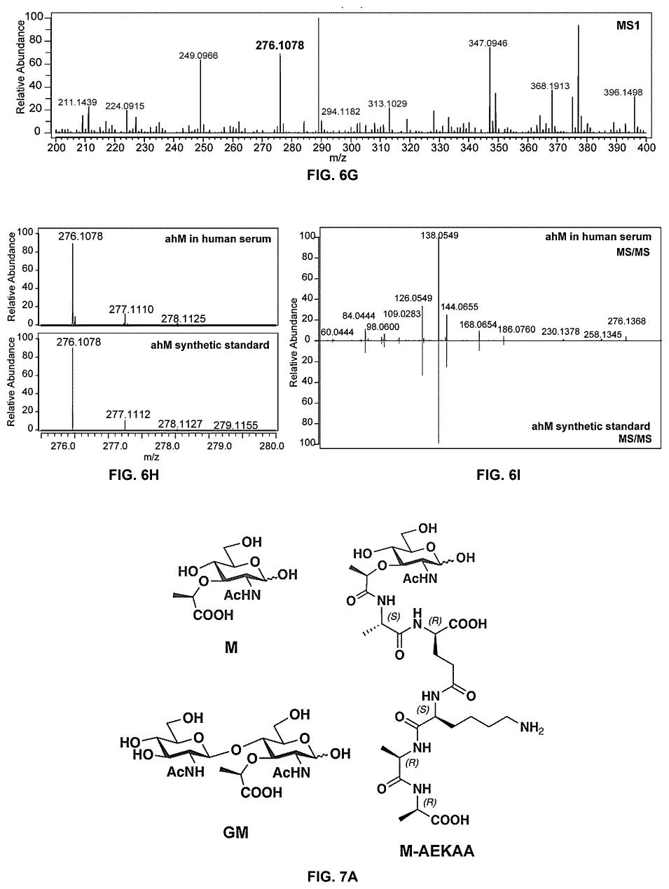

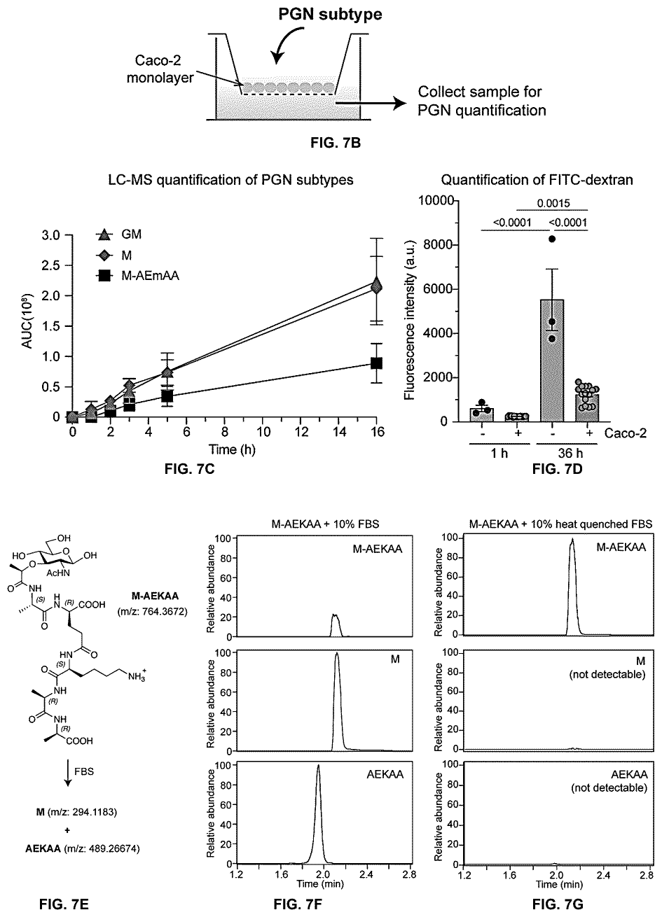

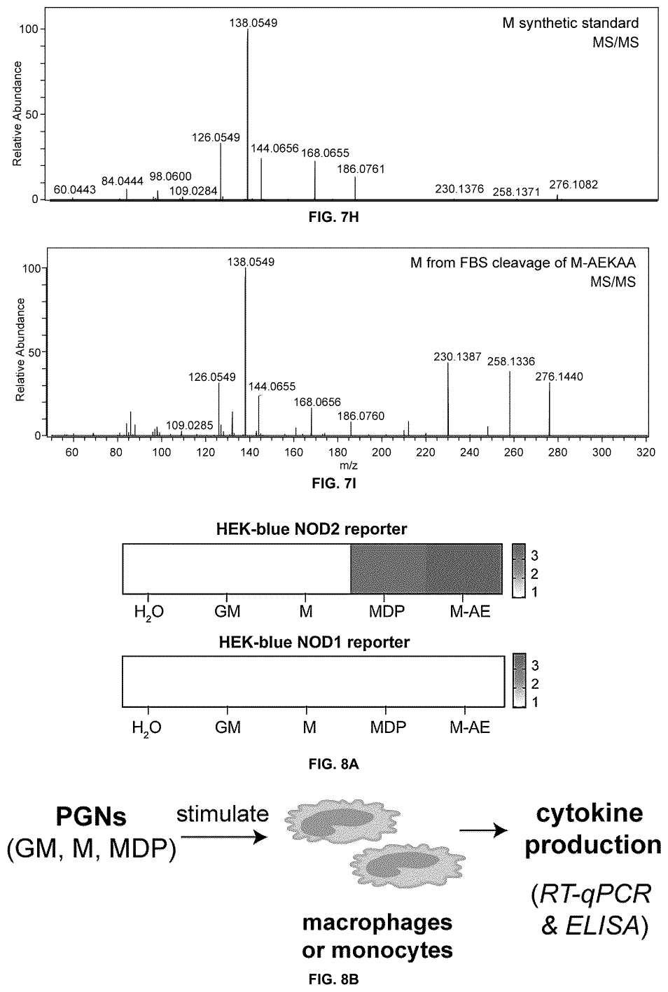

Profiling serum PGNs proved technically challenging due to low signal-to-noise ratios in LCMS/MS analysis. Nonetheless, in two different cohorts of pooled sera from healthy human individuals and in commercial FBS, PGN-derived monosaccharides M and ahM was identified, but not any muropeptides (FIG. 6A-61). The presence of M and ahM in serum coincides with their prominence as the two most abundant PGNs in the host gut (FIG. 4A-4C), while the striking difference in the overall abundance and diversity of soluble PGNs between the gut and serum suggests that the systemic dissemination of gut microbiota-derived PGNs is a selective process. In a Caco-2 monolayer-coated transwell system, PGN saccharides M and GM was shown translocated significantly faster than the larger muropeptide M-AEKAA (FIG. 7A-7D). Furthermore, it was reasoned that the absence of intact muropeptides in sera may be attributed to peptidoglycan-processing enzymes encoded by the host.[21] For instance, mammalian PGRP2 is a serum amidase that specifically cleaves the N-acetyl-muramyl-L-Ala bond of soluble PGNs,[22] whose activity may deplete muropeptides and contribute to the PGN derived monosaccharides in host sera. Consistently, it was shown that the incubation of 10% FBS with the muropeptide M-AEKAA reliably yields the expected degradation products: M and AEKAA (FIG. 7E-7I). Taken together, the LC-MS/MS profiling workflow reveals that saccharide moieties are the predominant gut PGN subtypes in hosts.

### Example 3: GM is Immunologically Bioactive Via NOD-Independent Mechanisms

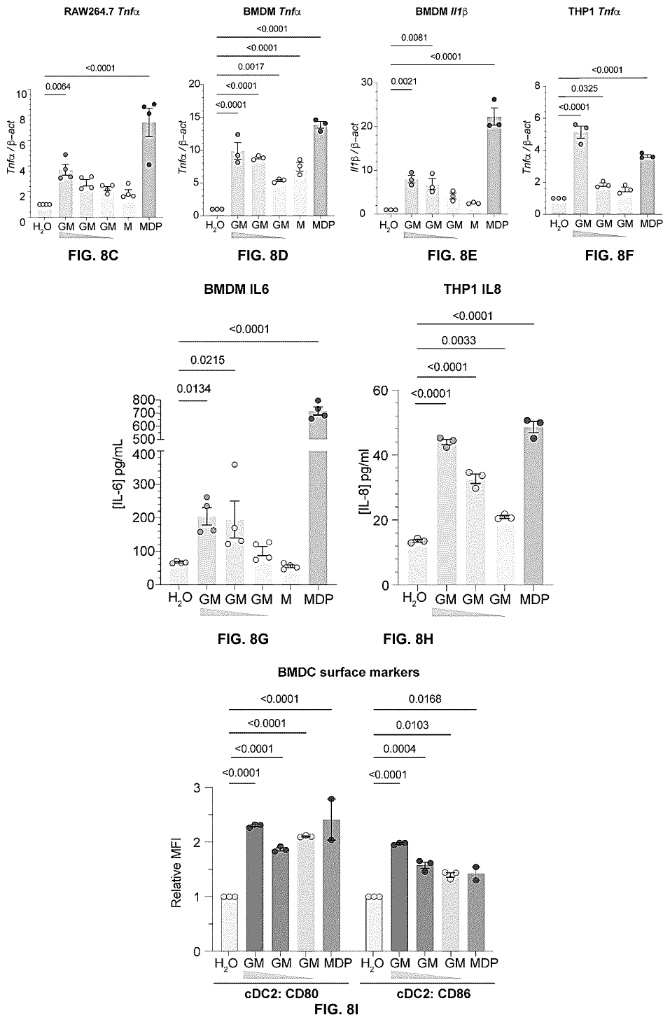

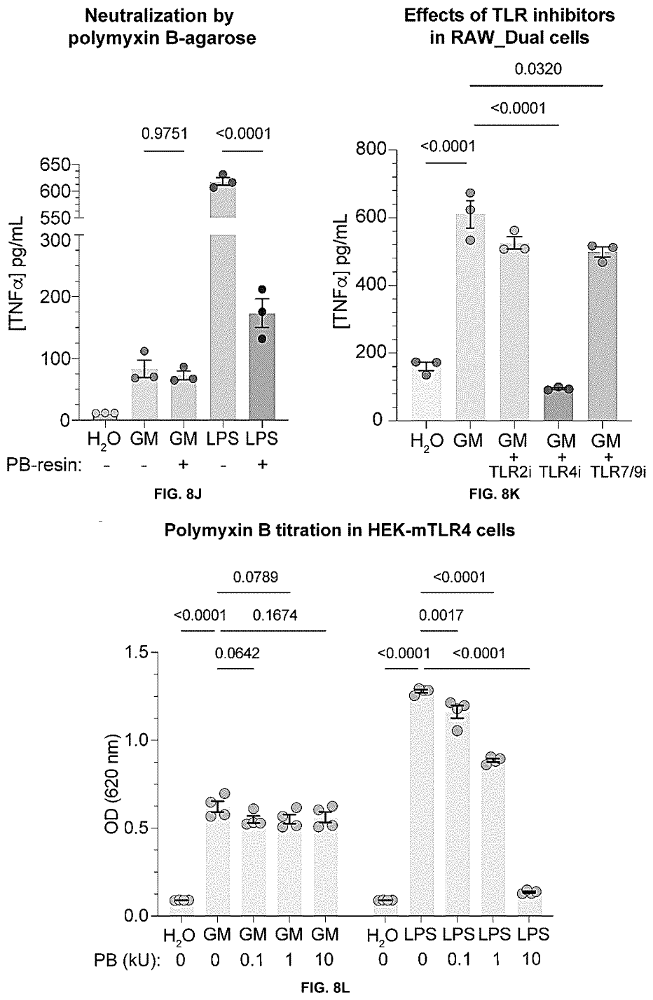

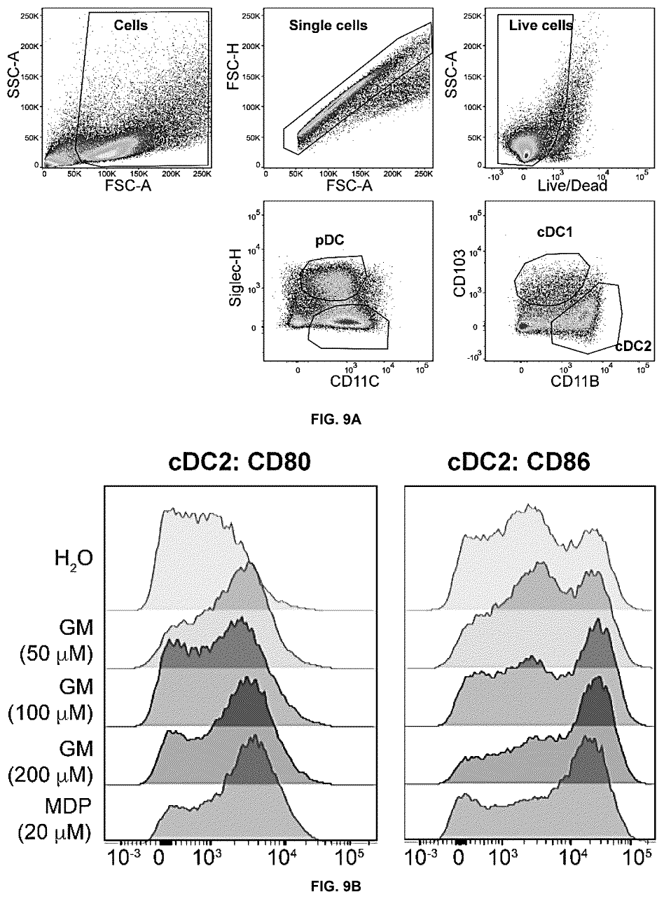

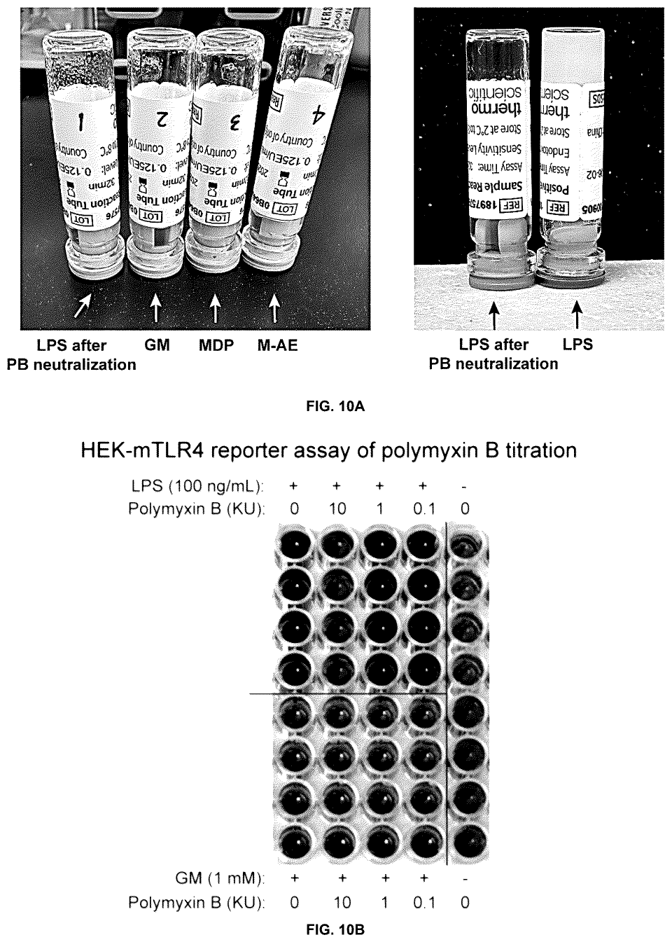

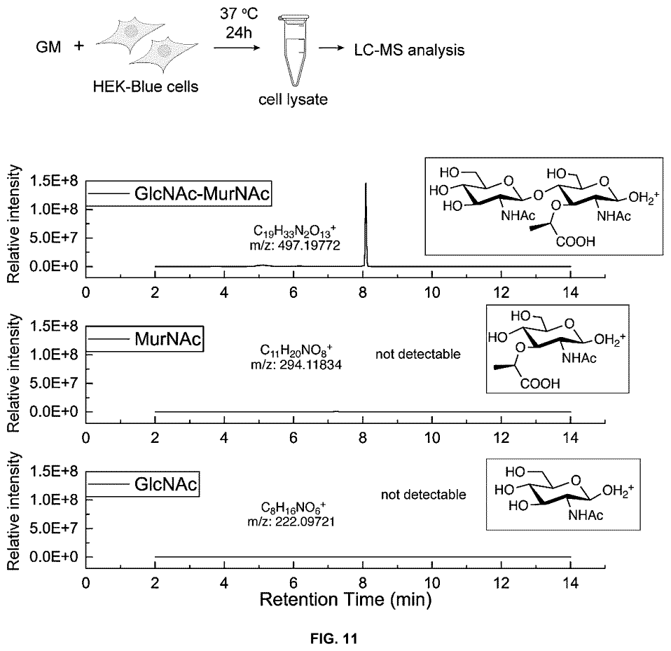

Intrigued by the abundance of gut microbiota-derived PGN saccharides in hosts at steady state, it was questioned if these natural subtypes exhibit any potential biological effects. To obtain sufficient PGNs of high purity for biological assays, a panel of PGNs of interest were chemically synthesized, including M-AE, GM, and M (FIG. 3C-3D). As mammalian NOD1/2 represents canonical PGN sensors, these PGNs were first assessed in HEK-Blue™ NOD reporter cells (FIG. 8A). As expected, the natural muropeptide M-AE potently elicited NOD2 activation similar to the model ligand MDP (i.e. M-AQ); yet the saccharide moieties, such as GM and M, are unable to activate NOD1/2, which is unsurprising given their lack of the required structural motifs for NOD1/2 recognition. At this juncture, it was sought to explore the potential effects of PGN saccharides in immune cells that express diverse PRRs, which are more physiologically relevant than the NOD reporter cells. Unexpectedly, the disaccharide GM exhibits dose dependent immuno-stimulatory activity in a range of immune cells, including murine macrophage RAW264.7, primary murine bone-marrow-derived macrophages (BMDM), and human monocytes (THP-1), as evidenced by the robust expression and production of proinflammatory cytokines such as TNFα and IL1b (FIG. 8B-8H). In comparison, the monosaccharide M appears less potent (FIG. 8C-8H), suggesting that the disaccharide structure may be crucial for immunoactivity. Furthermore, GM upregulates CD80/86 surface marker expressions in the type-II conventional DC (cDC2) population of murine BMDC (FIG. 8I, FIG. 9A-9B). Its ability to promote dendritic cell maturation ex vivo suggests a potential role in regulating adaptive immune responses in hosts. Importantly, it was confirmed that the synthetic GM is endotoxin-free, as it tested negative in the LAL assay and its immunological effect unaffected by polymyxin B neutralization (FIG. 8J, 8L, and FIG. 10A-10B). In addition, no glycosidase-cleaved monosaccharides (G and M) were detected in GM-treated host cells, indicating that the immunoactivity likely originates from the intact disaccharide GM rather than its degradation products (FIG. 11). With these observations, it was concluded that the disaccharide GM is a bioactive PGN motif that acts via NOD1/2-independent pathways.

### Example 4: TLR4 is Essential for GM-Triggered Immune Responses

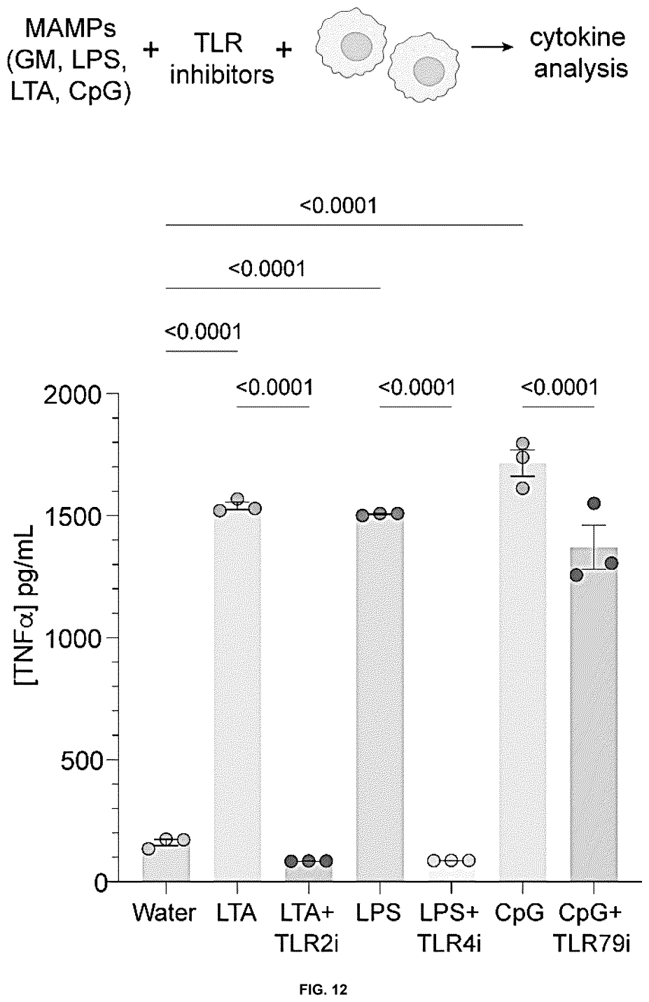

Apart from the NOD sensors, toll-like receptors (TLRs) represent another important class of mammalian PRRs that detect various MAMPs, many of which contain glycan motifs To assess whether GM is recognized by TLRs in immune cells, RAW264.7 cells were pretreated with a panel of small-molecule TLR inhibitors, then stimulated them with GM and analyzed cytokine levels in the culture supernatant using ELISA. Remarkably, the addition of TAK-242, a potent and selective TLR4 inhibitor, drastically suppressed GM-triggered TNFa production in RAW264.7 cells, while inhibition of TLR2 or TLR7/9 did not interfere with GM's bioactivity (FIG. 8K and FIG. 12). These results implicate TLR4, a well-recognized innate immune receptor of bacterial LPS, in sensing the gut microbiota-derived disaccharide GM. Importantly, it was demonstrated that the bioactivity of GM in HEK-Blue™-mTLR4 reporter cells is unaffected by polymyxin B titration, which effectively neutralizes LPS/endotoxin (FIG. 8L). This confirms that the observed TLR4-dependent immune effects of GM are not due to trace endotoxin contaminants.

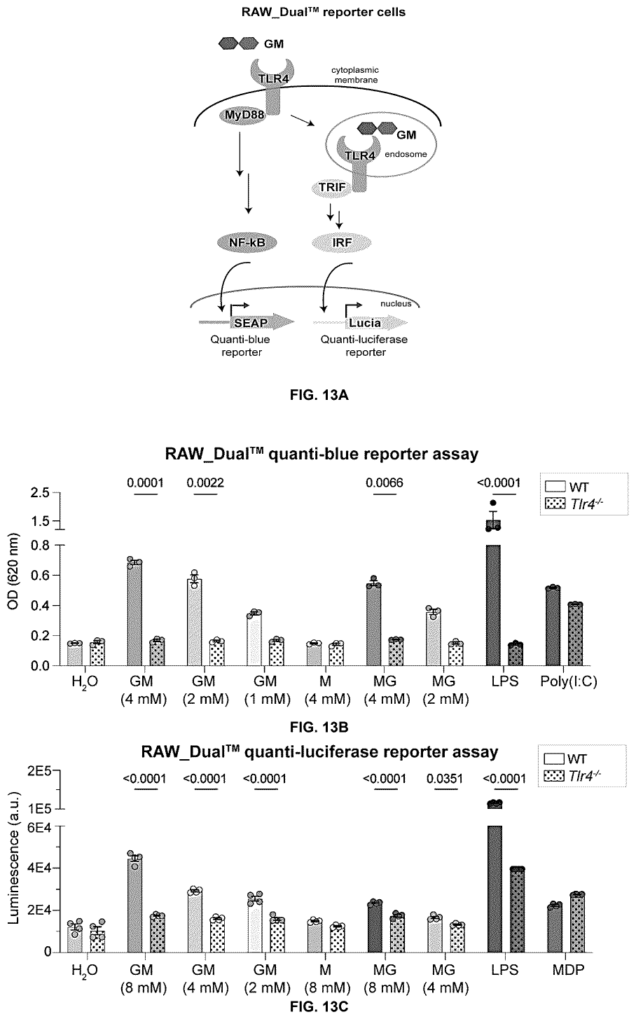

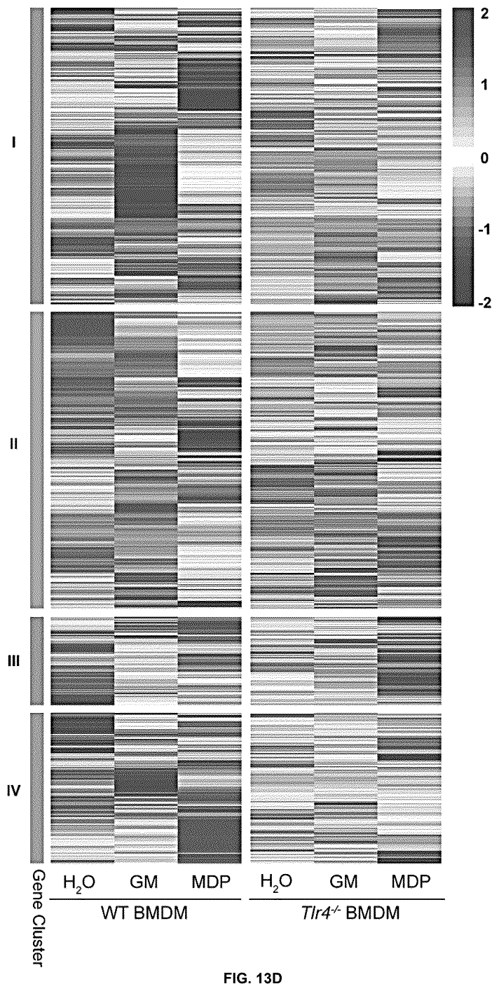

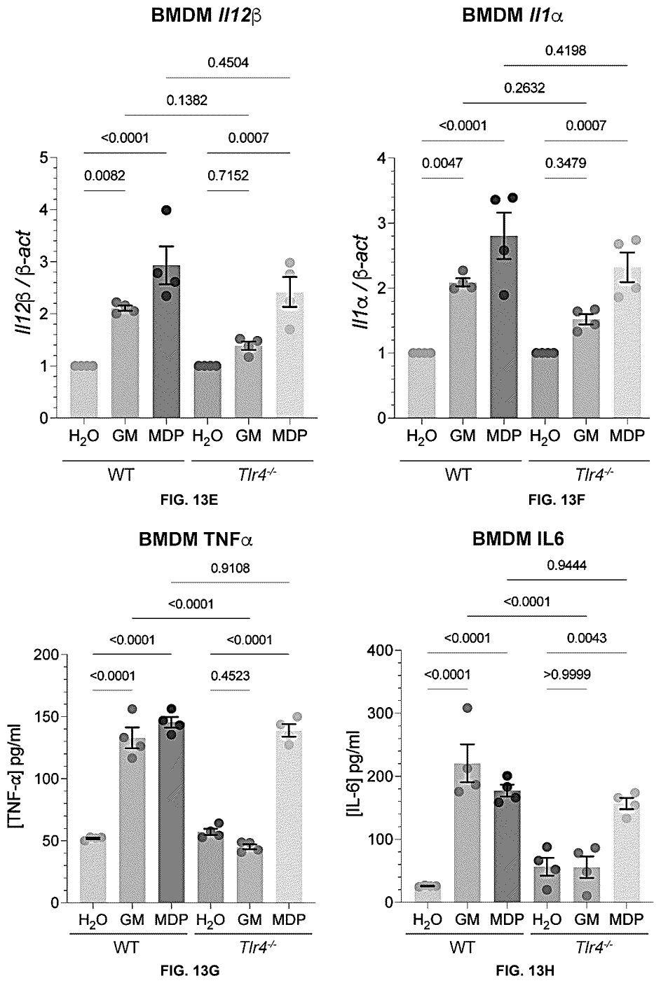

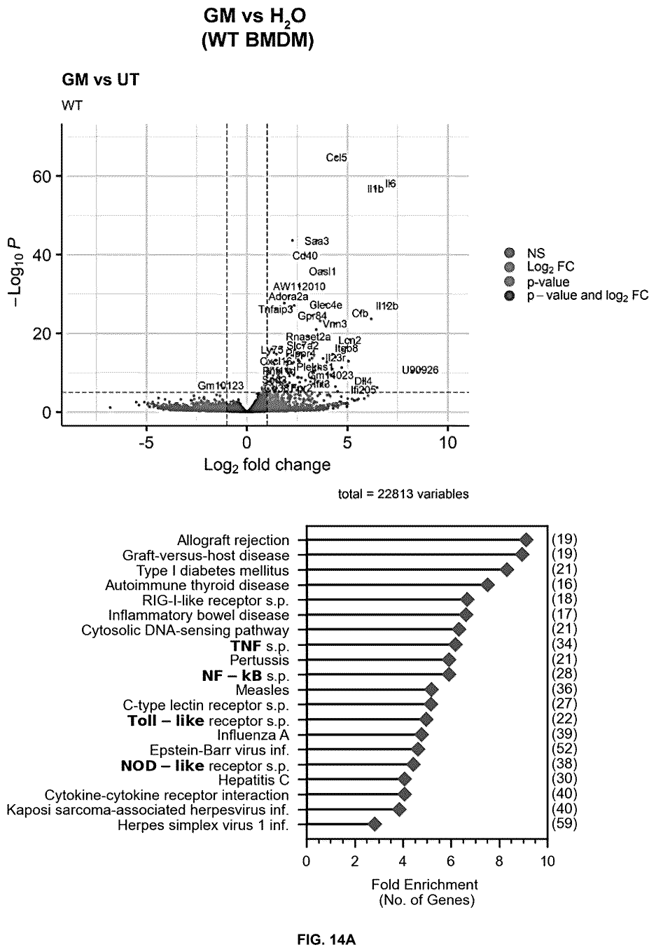

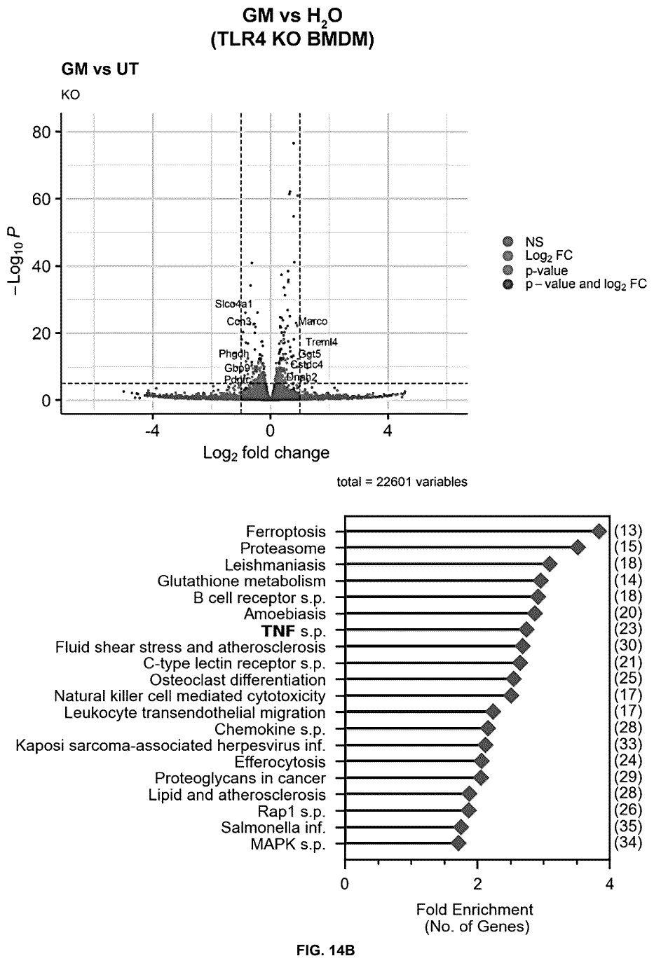

To conclusively establish the role of TLR4 in the immunostimulatory effects of GM, RAW-Dual™ wildtype (WT) and Tlr4−/− reporter cells for assays were next utilized. Notably, TLR4 activation is known to trigger two distinct signalling transduction pathways, NF-κB- and IRFdependent pathways, both of which can be expeditiously evaluated in parallel via colorimetric and luciferase readouts in the dual reporter cells (FIG. 13A). As expected, GM induces dose-dependent activation of both NF-κB and IRF signalling in WT cells but exhibits minimal activity in Tlr4−/− cells, which supports the essential role of TLR4 in GM-induced immune responses. Interestingly, the regio-isomer MurNAc-GlcNAc (MG) shows similar TLR4-dependent immune activation (FIG. 13B-13C).

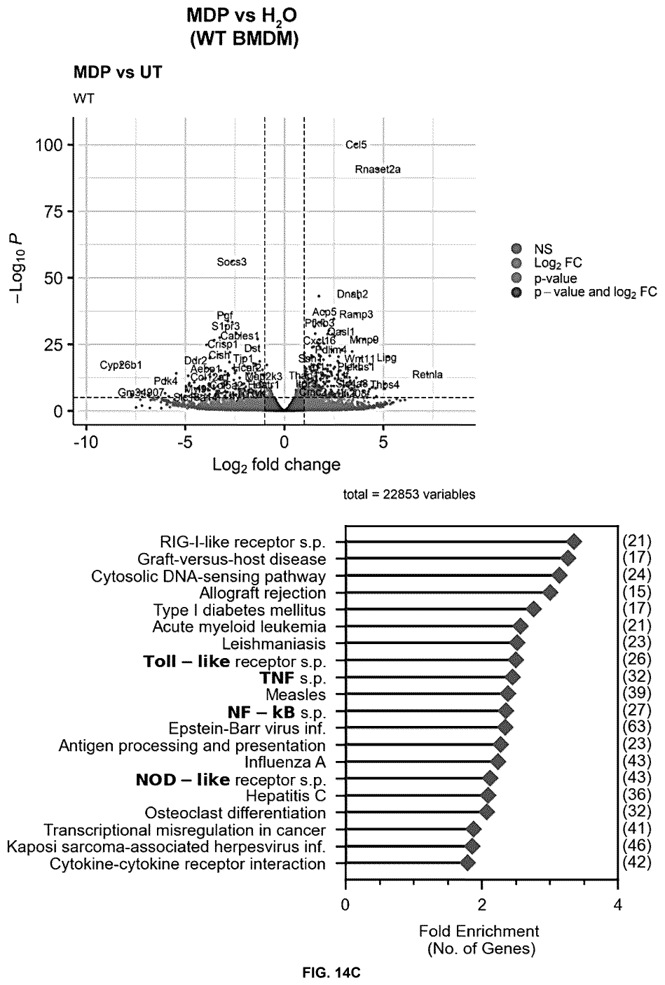

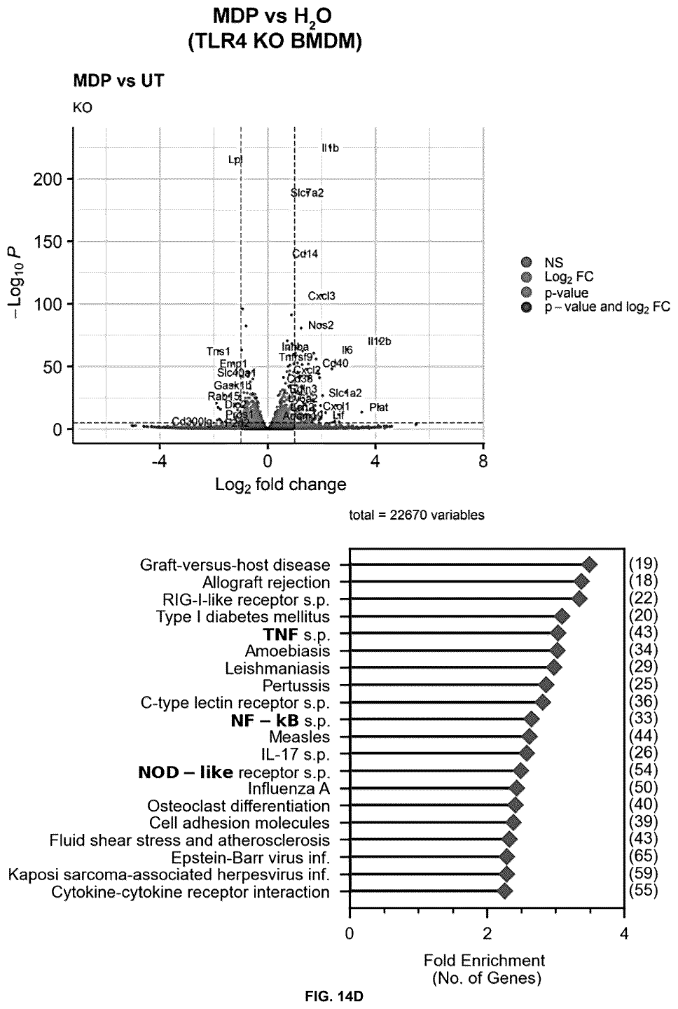

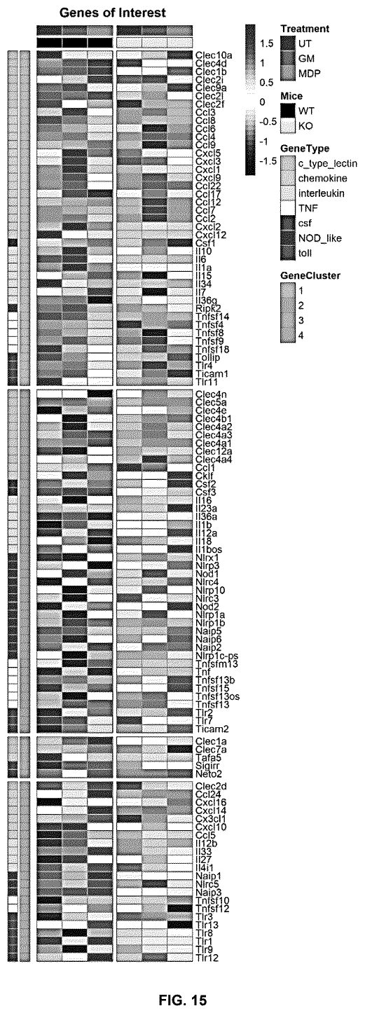

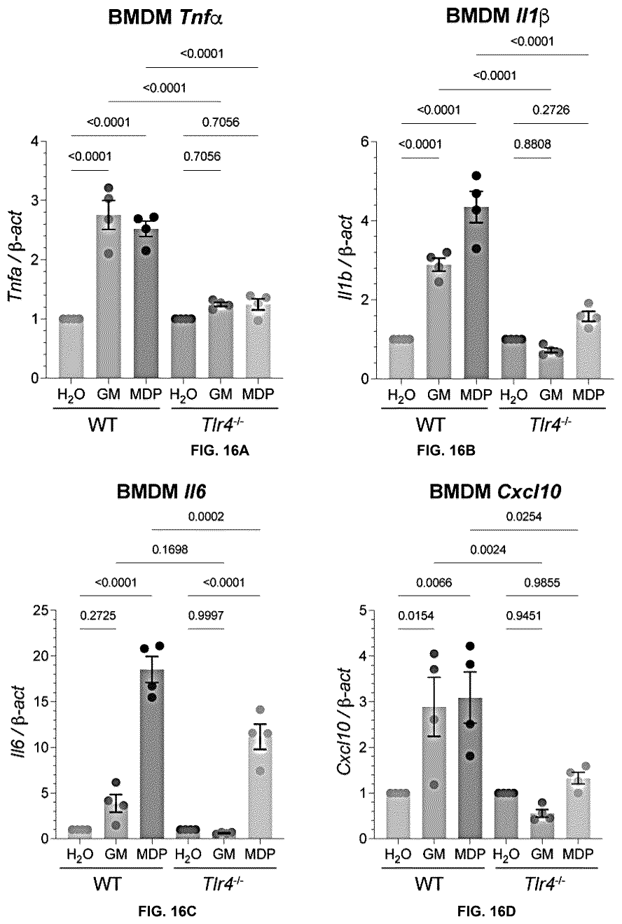

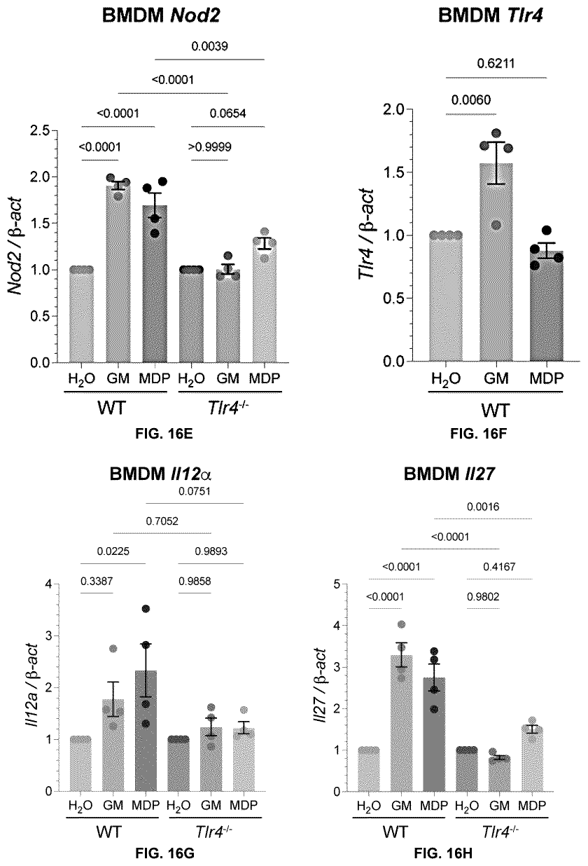

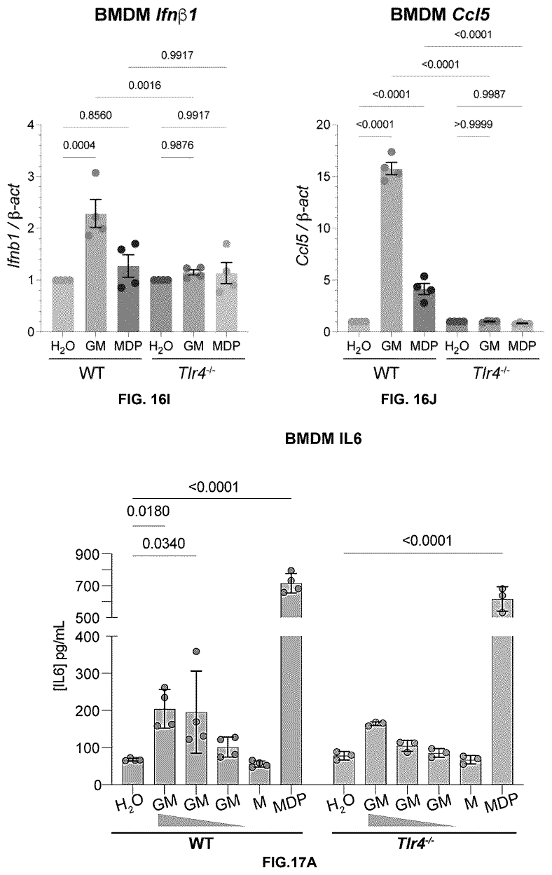

To gain deeper insights into the GM-induced transcriptional effects that are TLR4-dependent, whole-genome RNA sequencing (RNAseq) were performed in WT and Tlr4−/− BMDMs that were stimulated with GM for 24 h. Gene expression levels were analyzed with DESeq2 (FIG. 13D). KEGG pathway analysis of the GM-treated WT BMDM (versus H₂O-treated cells) identified several upregulated pathways associated with immune response and autoimmune diseases, such as Toll-like receptor signaling, NF-kB signaling pathway, and inflammatory bowel diseases, which are not enriched in the GM-treated Tlr4−/− BMDMs (FIG. 14A-14B). Specifically, a large number of cytokine and chemokine genes, including Il12a/b, Il6, Il1a/b, Csf3, Ccl5, and Cxcl1, are significantly upregulated in GM-treated WT BMDMs compared to the H₂O treated controls. In contrast, GM stimulation does not induce the expression of these genes in Tlr4−/− BMDMs (FIG. 15). The transcriptional changes of cytokines and chemokines were further validated by RT-qPCR (FIG. 13E-13F, FIG. 16A-16J). Notably, overexpression of tlr4 was also observed in the GM-treated BMDMs (FIG. 16A-16J). Collectively, these results confirm that TLR4 is indispensable in mediating the immunological effects of gut microbiota-derived disaccharide GM.

Additionally, RNA-seq data from WT and Tlr4−/− BMDMs treated with MDP was also analyzed, the model NOD2 ligand (FIG. 13D). As expected, MDP-induced pathways—including TNF signaling, NOD-like receptor signaling, and NF-kB signaling—are similarly enriched in WT and Tlr4−/− BMDMs (FIG. 14C-14D). Upon closer analysis of cytokines and chemokines, it was observed that while many genes, such as Il12 and Il6, are highly stimulated by MDP in both types of cells, a subset of MDP-induced genes, including Ccl5, Cxcl10, and Tnfa, appear to be TLR4-dependent (FIG. 13E-13F, FIG. 16A-16J). This intriguing discovery suggests that TLR4 may also play a role in modulating MDP-triggered NOD2 signaling, which was previously unrecognized. However, while MDP induces overexpression of nod2 in BMDMs as expected, it does not upregulate Tlr4 (FIG. 16A-16J).

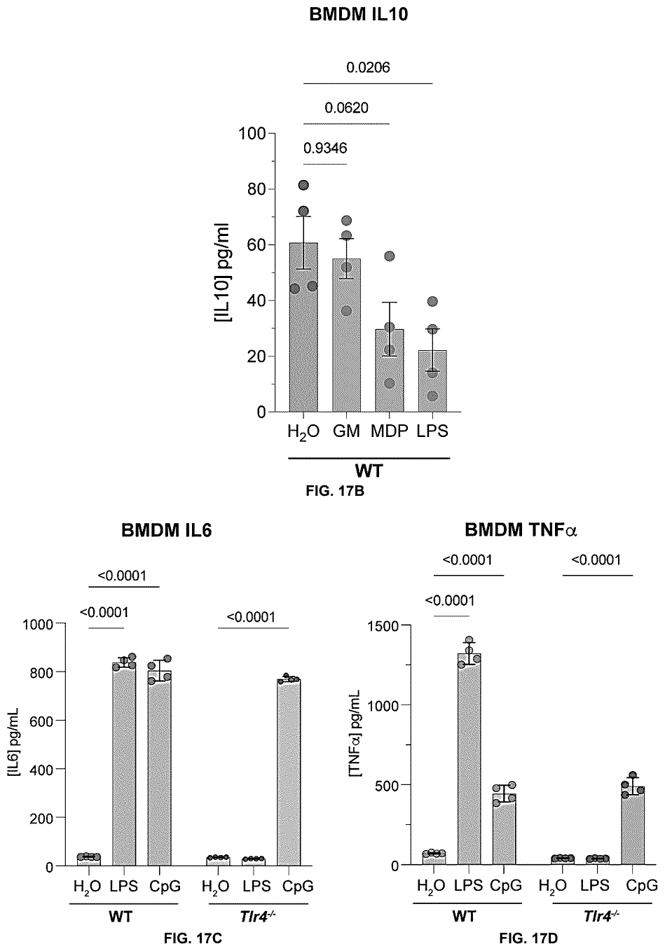

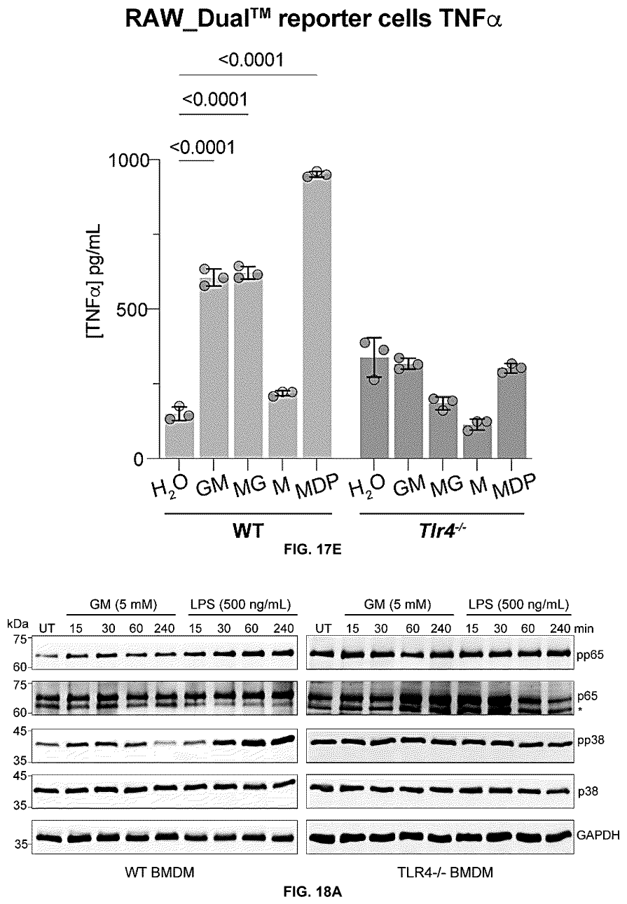

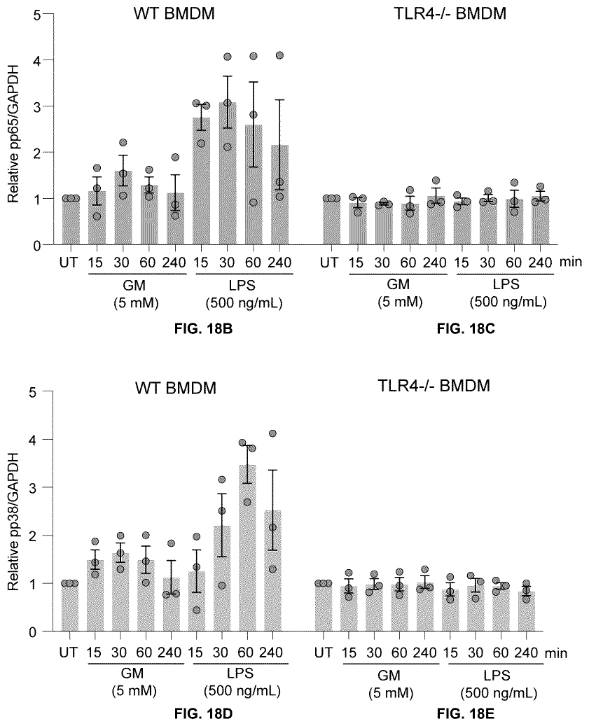

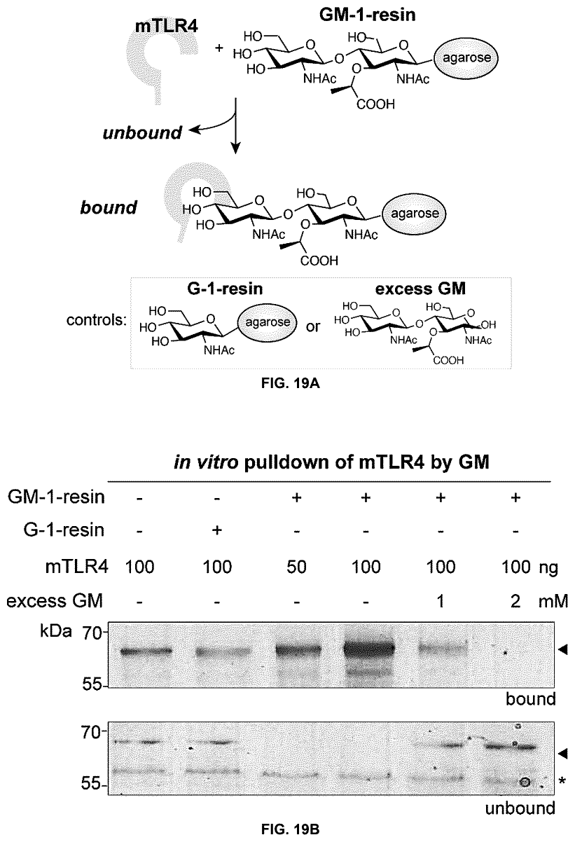

Lastly, cytokine production in treated Tlr4−/− BMDMs was also evaluated by ELISA. The disaccharide GM, like LPS, fails to elicit any significant level of TNFa and IL6 in Tlr4−/− BMDMs; in contrast, other ligands such as CpG and MDP still robustly trigger proinflammatory cytokine production in Tlr4−/− BMDM, similar to their effects in WT BMDMs (FIG. 13G-13H, FIG. 17A-17E). Of note, the lack of immune-stimulatory response in GM-treated Tlr4−/− BMDMs was paralleled with the complete absence of p65 and p38 phosphorylation, which was in contrast to the mild but robust increase in phosphorylation in GM-treated WT BMDMs (FIG. 18A-18E).

### Example 5: The Disaccharide GM Directly Binds to TLR4

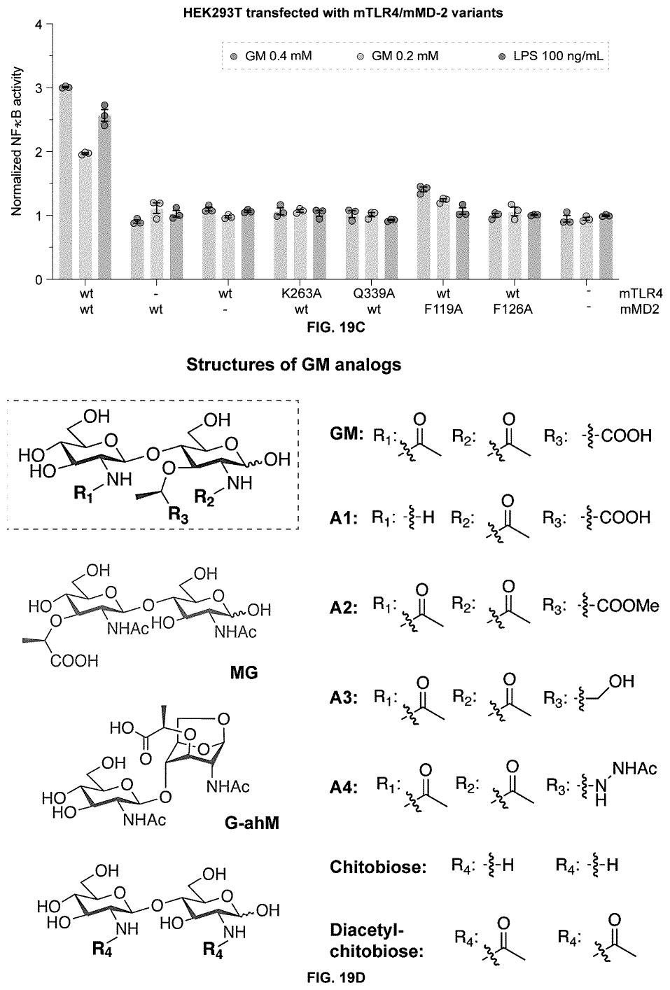

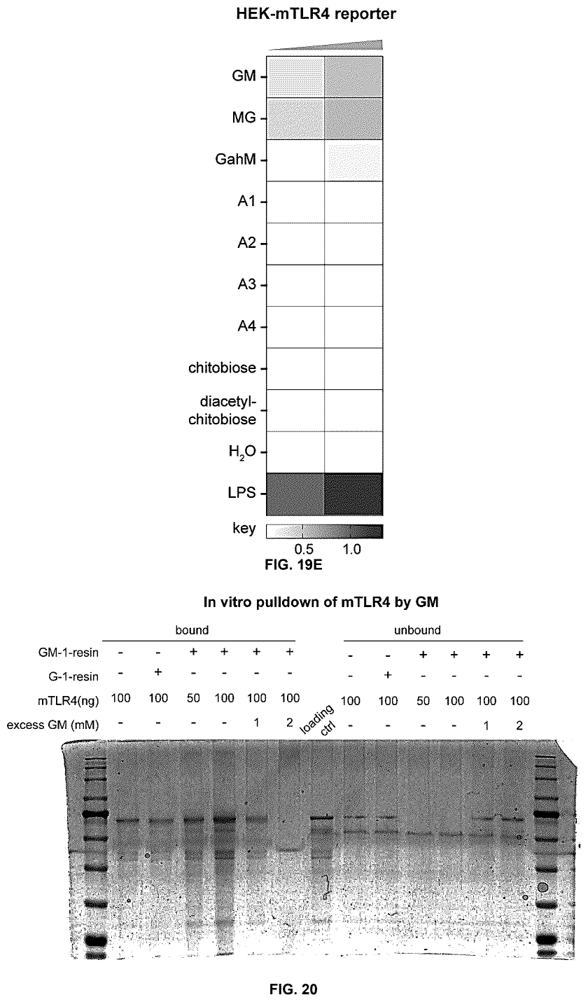

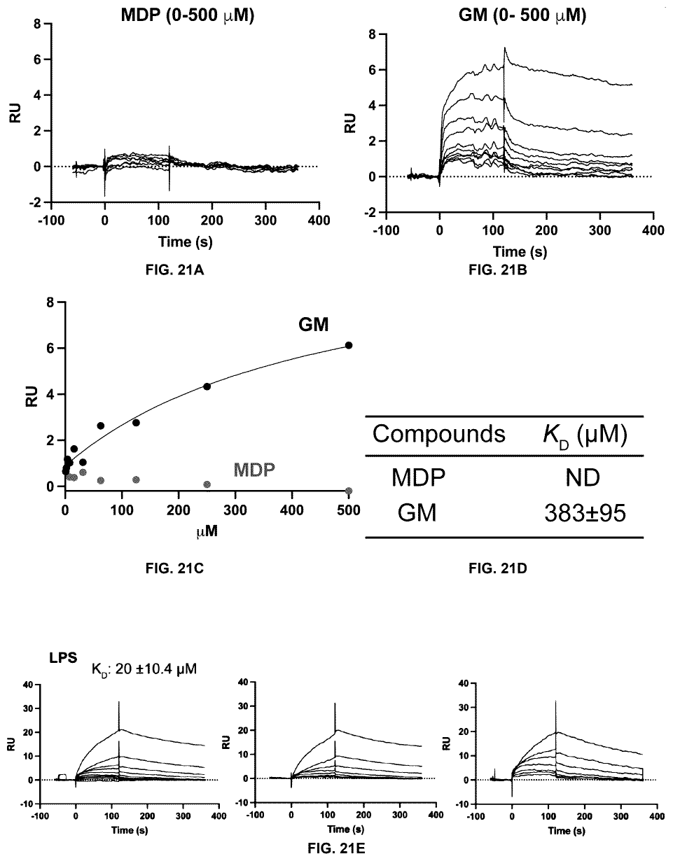

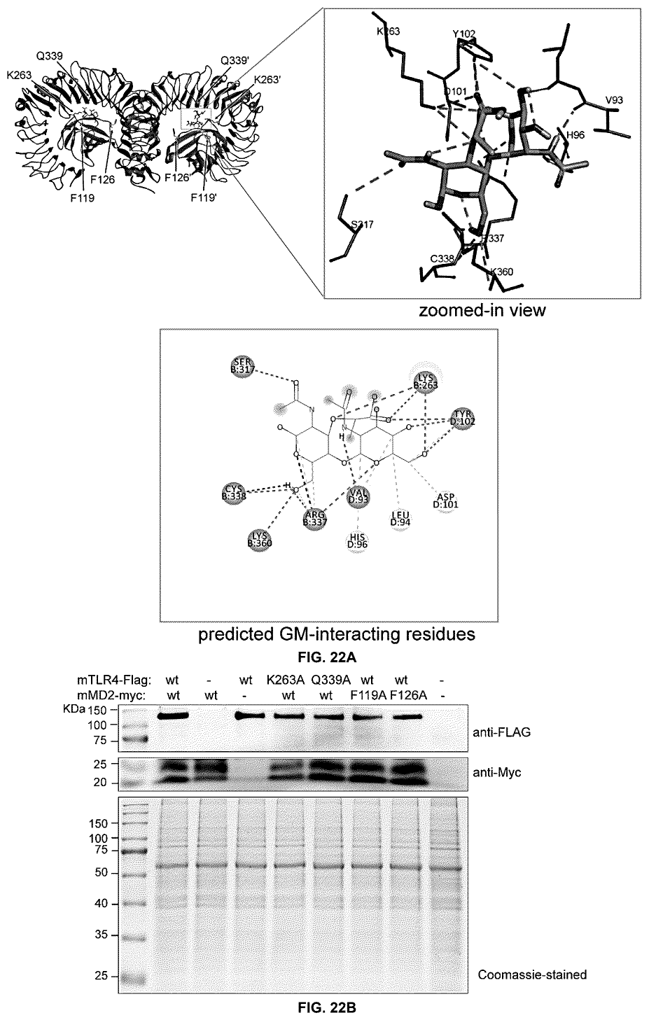

To conclusively establish TLR4 as the putative receptor for the disaccharide GM, biochemical evidence of GM binding to TLR4 was sought. First, in an in vitro pulldown assay, it was demonstrated that the immobilized GM-1 (GM-1-resin) effectively enriched recombinant mTLR4 protein in a dose-dependent manner, whereas the control resin immobilized with GlcNAc (G-1-resin) did not exhibit specific interaction with mTLR4. Pre-incubation of mTLR4 with excess GM as competitors led to a dose-dependent decrease in the amount of mTLR4 bound to GM-1-resin (FIG. 19A-19B and FIG. 20). To solicit additional evidence for direct GM-mTLR4 interaction, the surface plasmon resonance (SPR) assay was next performed (FIG. 21A-21E), in which LPS binds to mTLR4 in a dose-dependent manner (with Kd of ˜20±10.4 μM) while MDP does not interact with it, serving as appropriate positive and negative control ligands that validate the robustness of the SPR setup. As anticipated, the disaccharide GM binds to mTLR4 in the SPR assay, with an estimated Kd of ˜383 μM. The low binding affinity of GM to mTLR4 in vitro is consistent with its mild immunostimulatory effects observed in the cellular assays. This data confirms GM is a TLR4 ligand.

### Example 6: MD-2 is Essential for TLR4 Recognition of Disaccharide GM

Of note, MD-2 is an essential co-receptor of TLR4 for cell surface sensing of LPS, where specific residues in TLR4/MD-2 are known to stabilize the hydrophobic lipid chains and the polar glycan core of lipid A. To investigate if MD-2 is also required for TLR4 recognition of GM, the NF-κB activity was evaluated in GM-stimulated HEK293T cells that transiently express either mTLR4, mMD-2, or both, where the expression levels of the transfected genes were validated by Western blot (FIGS. 19C and 22A-22B). Similar to LPS, GM exhibits dose-dependent activation of NF-κB signaling only in cells that co-express mTLR4 and mMD-2, indicating MD-2 is essential for GM recognition by TLR4. Considering GM potentially resembles the disaccharide core of Lipid A, it is predicted that the TLR4/MD-2 residues involved in polar interactions are likely crucial for GM recognition. As expected, mutations of mTLR4_K263 and Q339, both of which form hydrogen bonds with the glycan core of Lipid A, effectively abolished GM-triggered NF-κB activity; whereas mutating mMD-2_F119, a residue that stabilizes the hydrophobic acyl chains in LPS, did not severely impair the activity of GM. In addition, mutation of mMD-2_F126, which blocks LPS-induced TLR4 dimerization, abolished NF-κB activation by both GM and LPS, suggesting that dimerization of the TLR4/MD-2 complex is required for GM-induced signal transduction. While several key molecular interactions in GM recognition were elucidated, a detailed understanding of how the disaccharide GM engages with the TLR4/MD-2 complex requires further structural investigations.

### Example 7: Structure-Activity-Relationship (SAR) Analysis of GM-Induced TLR4 Signaling

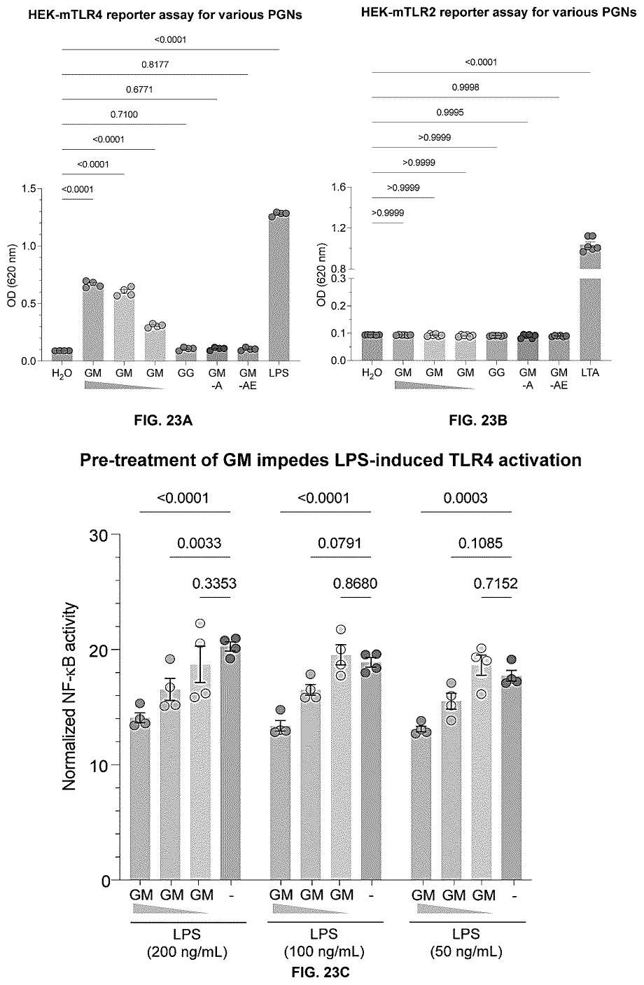

Given the mild stimulation by GM and its weak affinity for TLR4, the specificity of TLR4 recognition for disaccharide ligands was questioned. With HEK-Blue™ mTLR4 reporter cells, a panel of synthetic disaccharides bearing various structural modifications was tested, including deacetylation of GlcNAc (A1), alterations of the lactoyl moiety on MurNAc (A2-A4), 1,6-anhydro-terminus (G-ahM), and the regioisomer MG, as well as chitobiose and diacetylated chitobiose of the natural fungal cell wall (FIG. 19D). Remarkably, only GM and MG stimulate TLR4, while other minor structural changes render the disaccharide inactive (FIG. 19E), suggesting that TLR4 recognition of gut microbiota-derived disaccharides is highly specific. In addition, it was found that disaccharide-muropeptides, such as GM-A and GM-AE, also do not induce TLR4 signaling (FIG. 23A). However, despite its strong activation of

TLR4, GM does not activate TLR2 reporter cells, further confirming its specificity as a TLR4 agonist (FIG. 23B). Lastly, it was demonstrated that GM antagonizes LPS stimulated TLR4 signaling in reporter cell, likely by competitively binding to the TLR4 receptor (FIG. 23C).

### Example 8: GM Mitigates DSS-Induced Colitis in Mice in a TLR4-Dependent Manner

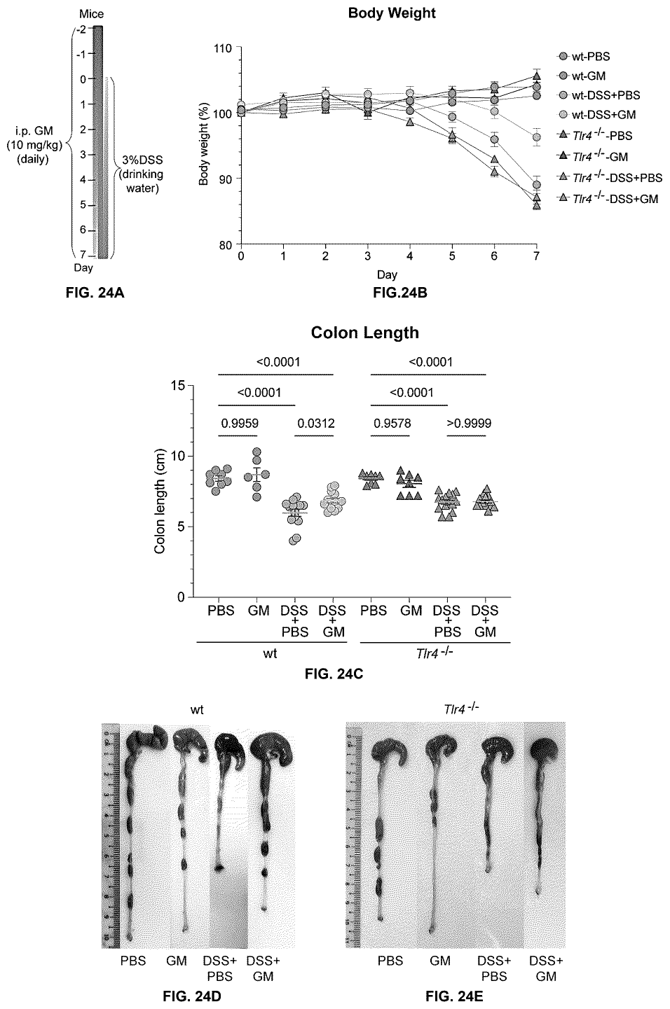

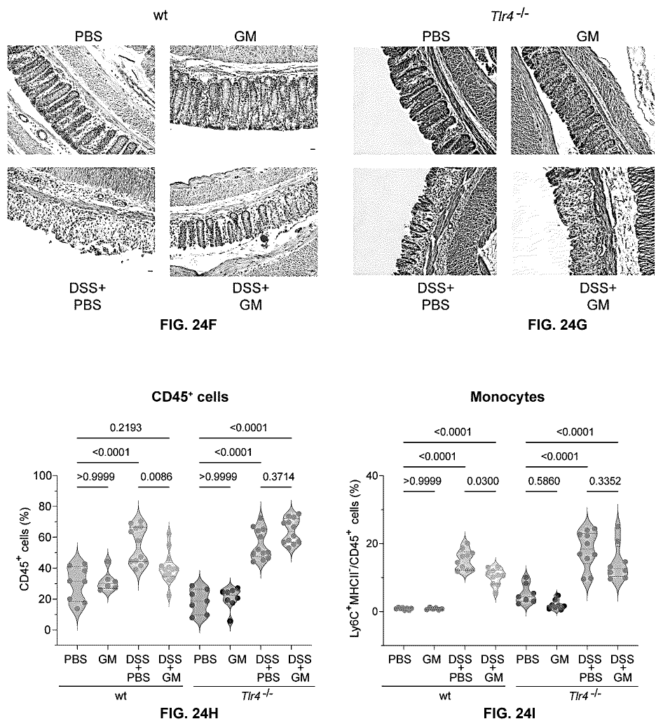

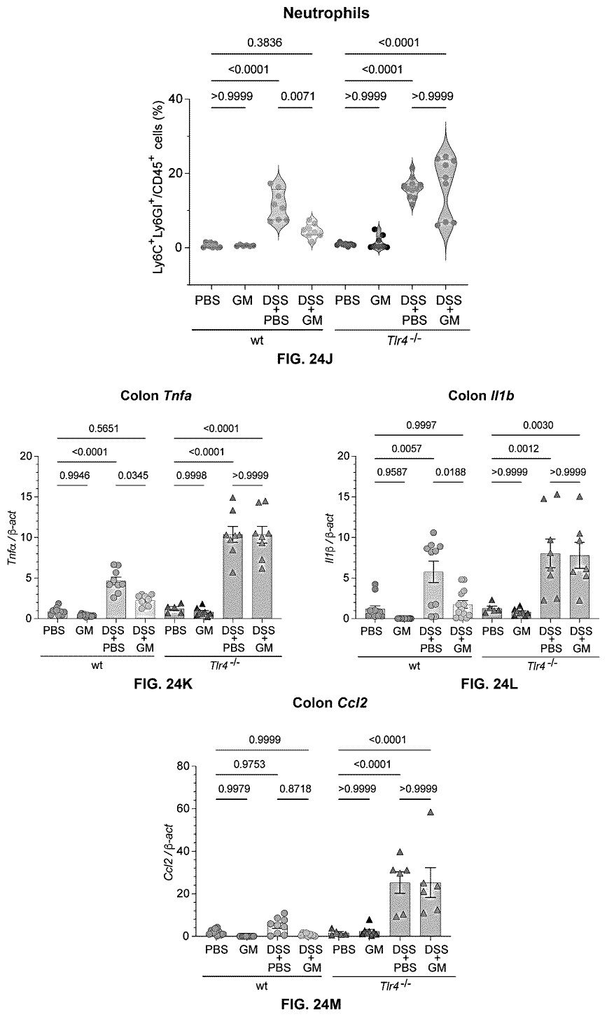

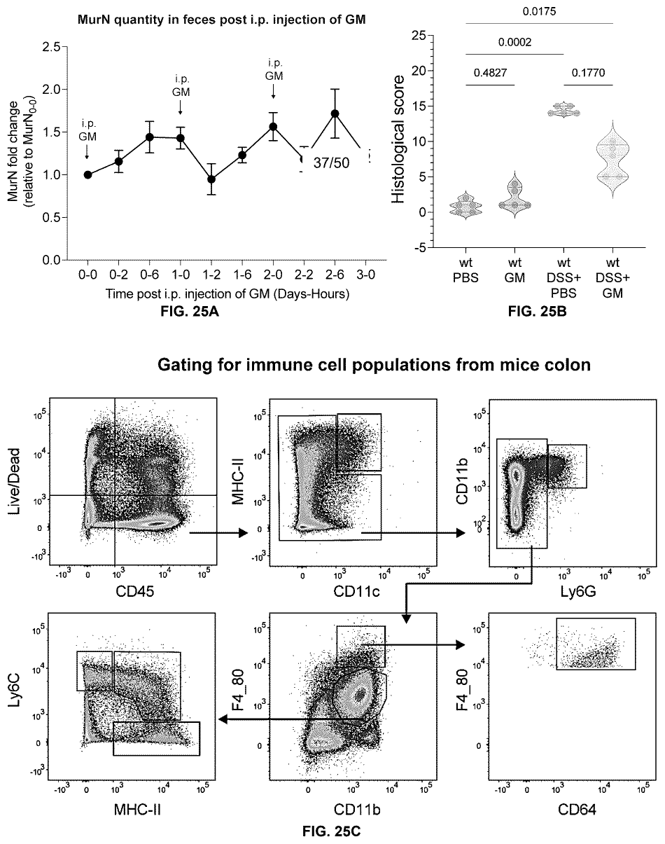

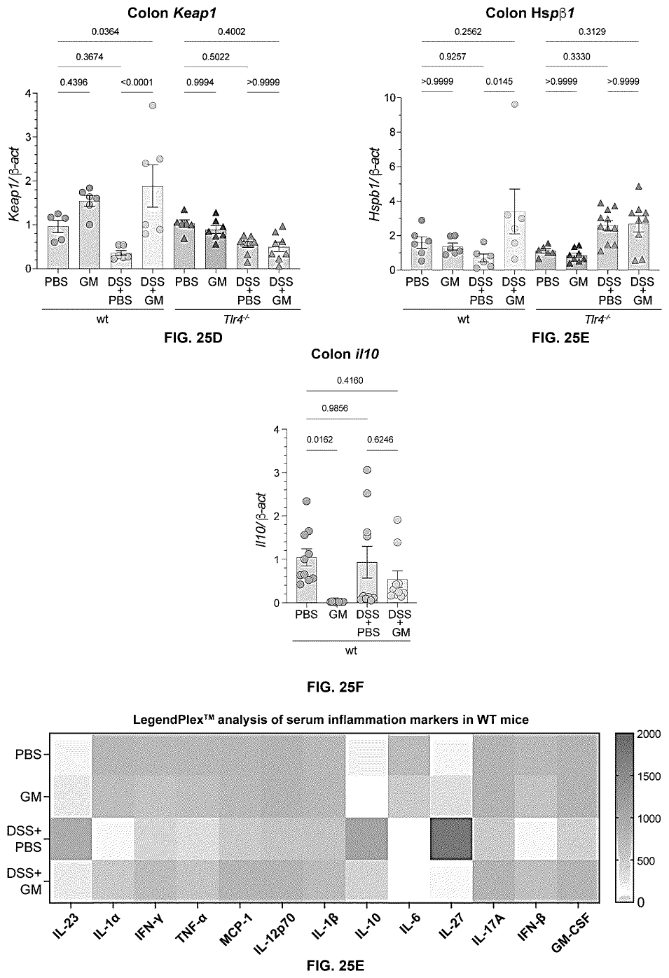

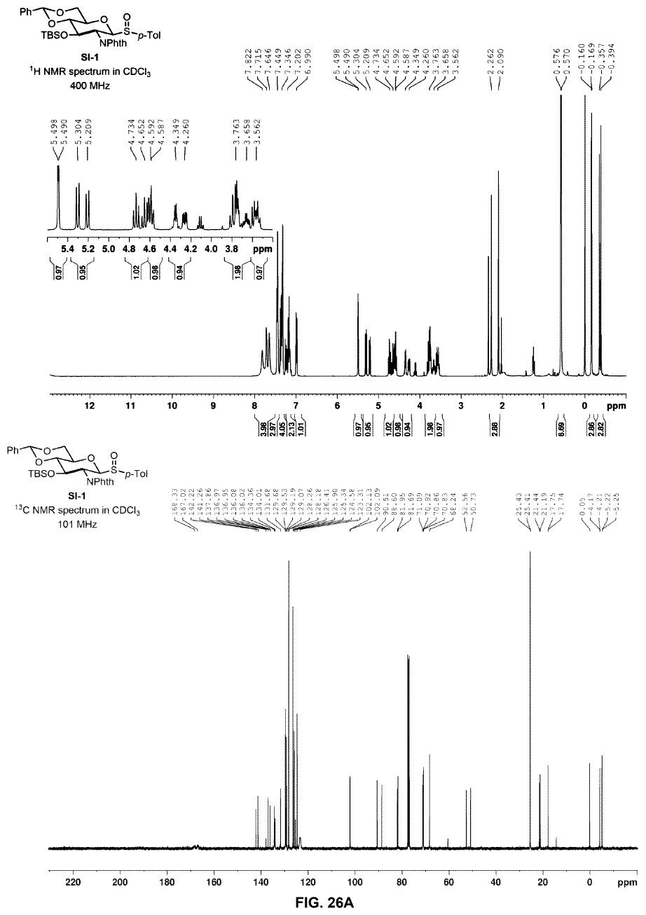

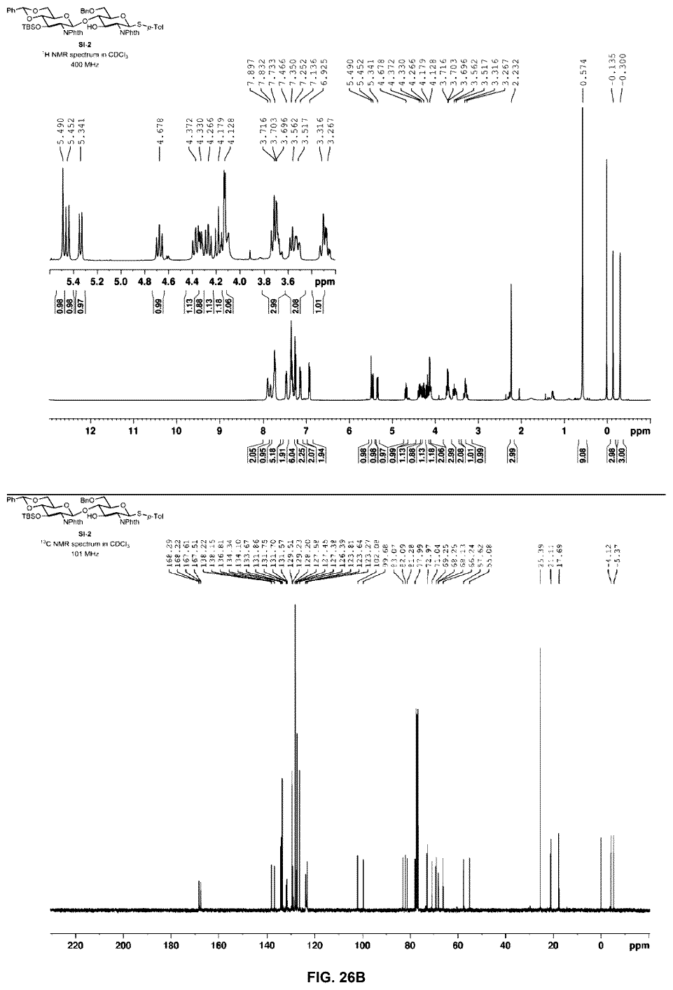

Upon establishing GM as a bioactive TLR4 ligand in vitro, it was asked whether this naturally released PGN disaccharide has any biological significance in vivo. Given previous work showing that MDP-mediated NOD2 signaling protects against DSS-induced colitis in mice,[23] the potential efficacy of GM in this context was explored. GM was administered intraperitoneally to mice daily for two days before and throughout a week during which 3% DSS was introduced into their drinking water. For control groups, mice were either injected with GM alone (i.e., no DSS treatment) or PBS buffer (i.e., no GM administration) followed by 3% DSS in their drinking water (FIG. 24A). Importantly, it was validated that intraperitoneal injection of GM temporally increased its abundance in the mouse gut, providing grounds for daily injections to maintain elevated levels of gut PGN saccharides (FIG. 25A). The administration of GM (without DSS treatment) did not result in any morbidity in mice. More gratifyingly, GM effectively alleviated DSS-induced colitis in WT mice, as evidenced by reduced body weight loss and colonic shortening (FIG. 24B-24E). In H&E-stained colonic tissue images, the GM-treated DSS group showed reduced epithelial erosion and mononuclear cell infiltration (FIG. 24F-24G, FIG. 25B). Correspondingly, infiltrating CD45+ immune cells, including monocytes and neutrophils, in the colonic lamina propria were significantly reduced in GM-treated DSS mice (FIG. 24H-24J, FIG. 25C). Consistently, colonic expressions of proinflammatory genes such as Tnfa, Il1b, and Ccl2 were significantly suppressed, supporting the effects of GM in attenuating colonic inflammations (FIG. 24K-24M). On the other hand, cytoprotective factors of colonic damage such as keap1 and hspb1 were upregulated in GM-treated colitis mice, although no significant change in il10 was observed (FIG. 25D-25F). To further assess systemic inflammation in mice, multiplex immunoassays were conducted to quantify 13 cytokines and chemokines in sera of the four groups of WT mice at the timeline endpoint (FIG. 25G). DSS-induced colitis mice manifested a significant global elevation of inflammatory markers, including IL-6, IFN-β, IL-27, IL-10, and IL-23. Remarkably, GM supplementation restored circulating proinflammatory markers to baseline levels, consistent with its ability to mitigate DSS-induced inflammations in mice. On the other hand, GM alone does not significantly alter circulating cytokines and chemokines in mice.

To determine whether GM's in vivo protective effects are TLR4-dependent, the aforementioned experiments in Tlr4−/− mice (FIG. 24A) were performed. In this case, GM administration failed to prevent weight loss and colonic shortening in DSS-administered Tlr4−/− mice (FIG. 24B-24E). In addition, similar levels of colonic crypt damage were observed in the colitis Tlr4−/− mice, regardless of GM supplementation (FIG. 24F-24G). The results of immune cell infiltration and colonic proinflammatory gene expression were consistent with the loss of GM's protective effects against DSS-induced colitis in Tlr4−/− mice (FIG. 24K-24M). No upregulation of cytoprotective responses in GM-treated colitis Tlr4−/− mice was observed (FIG. 25D-25F). Collectively, this data suggests that the gut microbiota-derived disaccharide GM maintains host gut homeostasis via TLR4-dependent mechanisms.

### Discussion

Using the LC-MS/MS-based PGN analysis platform, it was discovered that 90% of the gut microbiota-derived PGNs in the host gut consist solely of saccharide moieties, while the remaining muropeptides are primarily of the mDAP type. Given that the gut bacterial peptidoglycome encompasses both Lys- and mDAP-type peptidoglycan from Gram-positive and Gram-negative bacteria, [7] it is intriguing that the soluble muropeptides in the host gut milieu appear mostly derived from Gram-negative gut bacteria. It is speculated that gut commensals such as Bacteroides spp. that possess mDAP-containing peptidoglycan could be the major contributors to gut PGNs. Moreover, the model ligand MDP (M-AQ) was not identified in hosts; instead, its non-amidated analogues, such as M-AE and GMAE, were found to be prevalent in the mice ceca. These natural muramyl dipeptides are likely the cleavage products of commensal bacteria-secreted peptidoglycan endopeptidases, such as SagA and LPH. Previous studies have shown that elevating the levels of these enzymes or their corresponding cleaved muropeptide products benefits gut homeostasis and enhances cancer immunotherapy in mice.[24] On the other hand, a minute amount of PGN monosaccharide (e.g. M and ahM) were only detected in healthy host sera, which could indicate selective systemic dissemination of gut PGNs or PGN degradation by serum amidase PGRP2.[22] Alternatively, circulating PGNs may be retained by serum proteins or carried by vesicular transporters, potentially evading detection by LC-MS/MS. Nevertheless, unlike other existing PGN detection assays that rely on protein recognition of specific motifs, the LC-HRMS/MS analysis is unbiased and directly identifies PGN structures in hosts.

Intrigued by the abundance of PGN saccharides that do not resemble classic NOD1/2 ligands in the host gut, the potential bioactivity of these non-canonical PGN moieties were investigated. It was previously reported that cytosolic GlcNAc released from the phagosome degradation of bacterial peptidoglycan is detected by hexokinase that leads to inflammasome activation in LPS-primed immune cells, unveiling a NOD1/2-independent PGN sensing mechanism in the host.[12] Conversely, MurNAc was also shown to suppress inflammation in LPS-induced macrophages in vitro.[25] Herein, it is demonstrated that the gut microbiota-derived disaccharide GM exerts mild immuno-stimulatory effects in the absence of LPS priming, under which condition the monosaccharides G and M are inactive. Importantly, it was established that GM acts as a TLR4 agonist, which directly binds to TLR4 and activates downstream NF-kB and IRF pathways. Of note, bacterial LPS is the canonical TLR4 ligand, whose Lipid A motif constitutes a β-1,6-linked glucosamine disaccharide with phosphate groups and multiple acyl chains. Apart from LPS, endogenous ligands such as heat shock proteins, hyaluronan, and monosodium urate crystals, as well as synthetic small molecules like neoseptins are reported to stimulate TLR4 signaling, demonstrating the structural diversity of TLR4 ligands.[26] The discovery of the gut microbiota-derived disaccharide GM as a TLR4 agonist expands the scope of natural TLR4 ligands in hosts. Structurally, GM may resemble the disaccharide core of Lipid A for TLR4 recognition; however, the lack of hydrophobic acyl chains in GM renders it a mild TLR4 agonist. Interestingly, the presence of a stem peptide in disaccharide (GM)-muropeptides, such as GM-A and GM-AE, effectively abrogates the TLR4-activating effects. Moreover, no TLR2 stimulation was observed by GM and these other PGN motifs. Hence, the fact that TLR4 selectively recognizes disaccharide PGNs (i.e., GM and its regioisomer MG) but not closely related fungal cell wall fragments (such as GG) underscores the potential biological significance of natural PGNs in gut microbiota-host interactions.

Notably, TLR4 activation by metabolites from gut commensal microflora under steady-state conditions is essential for maintaining gut homeostasis and protecting against colonic injury. For instance, LPS supplementation effectively rescues the severity of DSS-induced colitis in commensal-depleted mice via TLR4 stimulation.[27] The discovery of the naturally abundant gut microbiota-derived disaccharide GM as a novel TLR4 ligand suggests its physiological relevance in hosts. Indeed, the administration of GM protects against DSS-induced colitis in mice via TLR4-dependent mechanism(s). Several hypotheses accounting for its in vivo protection is suggested. First, GM, as a mild TLR4 agonist, may act to prime TLR4 to downregulate its inflammatory responses to subsequent triggers in hosts. Supportively, it was observed that GM antagonizes LPS-induced activation of TLR4-mediated NF-κB signaling in reporter cells in vitro. Second, colonic epithelium TLR4 activation confers cytoprotection and repair against DSS-induced tissue injury and damage.[27] To this end, it was confirmed that GM supplementation indeed upregulated cytoprotective genes such as keap1 and hspb1 in colonic tissues of the DSS-treated wildtype but not Tlr4−/− mice. However, GM did not seem to promote the anti-inflammatory cytokine IL10 in vitro and in vivo. Lastly, GM-induced TLR4 activation may in turn regulate other signaling pathways to provide protective effects in vivo. For example, MDP activation of NOD2 suppresses DSS-induced colitis in mice via the induction of negative regulator IRF4.[23] Intriguingly, transcriptomics analysis uncovered the intricate effects of TLR4 on MDP-mediated NOD2 signaling, as indicated by the reduced expressions of specific genes in MDP-treated Tlr4−/− BMDMs compared to treated WT BMDMs. Extrapolating from these in vitro observations, it is hypothesized that GM supplementation stimulates TLR4 signaling, which may promote NOD2 activation by endogenous gut microbiota-derived muropeptides like M-AE and GM-AE to protect against DSS-induced colitis in mice. Consistently, GM triggers Nod2 overexpression in WT BMDMs but not in the Tlr4−/− counterparts, implying that TLR4-mediated NF-κB signaling may regulate NOD2 expression. From a therapeutic perspective, the gut microbiota-derived disaccharide GM—a mild TLR4 agonist that does not induce acute inflammation or morbidity in mice—emerges as a promising candidate for postbiotics or adjuvants. Fine-tuning the TLR4-mediated immune responses holds significant potential in the treatment of gut associated inflammatory diseases.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein. The compounds, compositions, uses and methods described herein are presently representative of preferred embodiments are exemplary and are not intended as limitations on the scope of the invention. Changes therein and other uses will occur to those skilled in the art which are encompassed within the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

1. Rooks, M. G., and Garre9, W. S. (2016). Gut microbiota, metabolites and host immunity. Nat Rev Immunol 16, 341-352. h9ps://doi.org/10.1038/nri.2016.42.
2. Bouskra, D., et al (2008). Lymphoid Ussue genesis induced by commensals through NOD1 regulates intesUnal homeostasis. Nature 456, 507-510. h9ps://doi.org/10.1038/nature07450.
3. Mazmanian, S. K., et al (2005). An immunomodulatory molecule of symbioUc bacteria directs maturaUon of the host immune system. Cell 122, 107-118. h9ps://doi.org/10.1016/j.cell.2005.05.007.
4. Pedicord, V. A., et al (2016). ExploiUng a host-commensal interacUon to promote intesUnal barrier funcUon and enteric pathogen tolerance. Sci Immunol 1, eaai7732. h9ps://doi.org/10.1126/sciimmunol.aai7732.
5. Scher, J. U., and Abramson, S. B. (2011). The microbiome and rheumatoid arthriUs. Nat.Rev. Rheumatol. 7, 569-578. h9ps://doi.org/10.1038/nrrheum.2011.121.
6. Wheeler, R., et al (2014). The biology of bacterial pepUdoglycans and their impact on host immunity and physiology. Cell Microbiol 16, 1014-1023. h9ps://doi.org/10.1111/cmi. 12304.
7. Vollmer, W., Blanot, D., and de Pedro, M. A. (2008). PepUdoglycan structure and architecture. FEMS Microbiol Rev 32, 149-167. h9ps://doi.org/10.1111/j.1574-6976.2007.00094.x.
8. Li, C., Liang, Y., and Qiao, Y. (2022). Messengers From the Gut: Gut Microbiota-Derived Metabolites on Host RegulaUon. Front Microbiol 13, 863407. h9ps://doi.org/10.3389/fmicb.2022.863407.
9. Irazoki, O., et al (2019). PepUdoglycan MuropepUdes: Release, PercepUon, and FuncUons as Signaling Molecules. Front Microbiol 10, 500. h9ps://doi.org/10.3389/fmicb.2019.00500.
10. Girardin, S. E., et al. (2003). Nod1 Detects a Unique MuropepUde from Gram-NegaUve Bacterial PepUdoglycan. Science 300, 1584-1587.h9ps://doi.org/10.1126/science.1084677.
11. Inohara, N., et al. (2003). Host recogniUon of bacterial muramyl dipepUde mediated through NOD2. h9ps://doi.org/10.1074/jbc.C200673200. ImplicaUons for Crohn's disease. J Biol Chem 278, 5509-5512.
12. Wolf, A. J., et al. (2016). Hexokinase Is an Innate Immune Receptor for the DetecUon of Bacterial PepUdoglycan. Cell 166, 624-636. h9ps://doi.org/10.1016/j.cell.2016.05.076.
13. Kwan, J. M. C., et al. (2024). In silico MS/MS predicUon for pepUdoglycan profiling uncovers novel anU-inflammatory pepUdoglycan fragments of the gut microbiota. Chem Sci 15, 1846-1859. h9ps://doi.org/10.1039/d3sc05819k.
14. Zurek, B., et al (2011). Cell-based reporter assay to analyze acUvaUon of Nod1 and Nod2. Methods Mol Biol 748, 107-119. h9ps://doi.org/10.1007/978-1-61779-139-0_7.
15. Huang, Z., et al. (2019). AnUbody neutralizaUon of microbiota-derived circulaUng pepUdoglycan dampens inflammaUon and ameliorates autoimmunity. Nat. Microbiol. 4, 766-773. h9ps://doi.org/10.1038/s41564-019-0381-1.
16. Huang, Z. et al. (2019). Antibody neutralization of microbiota-derived circulating peptidoglycan dampens inflammation and ameliorates autoimmunity. Nat. Microbiol. 4, 766-773. 10.1038/s41564-019-0381-1.
17. Park, B. S., et al (2009). The structural basis of lipopolysaccharide recognition by the TLR4-MD-2 complex. Nature 458, 1191-1195. 10.1038/nature07830.
18. Kantoci, D., et al. Carbohydr. Res. 1987, 162, 227.
19. Bastos, P. A. D., et al (2021). Uptake, recogniUon and responses to pepUdoglycan in the mammalian host. FEMS Microbiol Rev 45, fuaa044.h9ps://doi.org/10.1093/femsre/fuaa044.
20. Bro9, A. S., and Clarke, A. J. (2019). PepUdoglycan O-AcetylaUon as a Virulence Factor: Its Effect on Lysozyme in the Innate Immune System. AnUbioUcs (Basel) 8, 94.h9ps://doi.org/10.3390/anUbioUcs8030094.
21. Dziarski, R., and Gupta, D. (2006). Mammalian PGRPs: novel anUbacterial proteins. Cell Microbiol 8, 1059-1069. h9ps://doi.org/10.1111/j.1462-5822.2006.00726.x.
22. Wang, Z.-M., et al. (2003). Human PepUdoglycan RecogniUon Protein-L Is an N-Acetylmuramoyl-L-alanine Amidase*. J Biol Chem 278, 49044-49052. h9ps://doi.org/10.1074/jbc.M307758200.
23. Watanabe, T., et al (2008). Muramyl dipepUde acUvaUon of nucleoUde-binding oligomerizaUon domain 2 protects mice from experimental coliUs. J Clin Invest 118, 545-559.h9ps://doi.org/10.1172/JCI33145.
24. Griffin, M. E et al (2021). Enterococcus pepUdoglycan remodeling promotes checkpoint inhibitor cancer immunotherapy. Science 373, 1040-1046. h9ps://doi.org/10.1126/science.abc9113.
25. Wu, Z., Pan, D., Guo, Y., and Zeng, X. (2015). N-acetylmuramic acid triggers anU-inflammatory capacity in LPS-induced RAW 264.7 cells and mice. J Funct Foods 13, 108-116. h9ps://doi.org/10.1016/j.jff.2014.12.048.
26. Yu, L., Wang, L., and Chen, S. (2010). Endogenous toll-like receptor ligands and their biological significance. J Cell Mol Med 14, 2592-2603. h9ps://doi.org/10.1111/j.1582-4934.2010.01127.x.
27. Rakoff-Nahoum, S., et al (2004).Recognition of commensal microflora by toll-like receptors is required for intesUnal homeostasis. Cell 118, 229-241. h9ps://doi.org/10.1016/j.cell.2004.07.002

1. A Toll-Like Receptor 4 (TLR4) agonist having a formula (I): 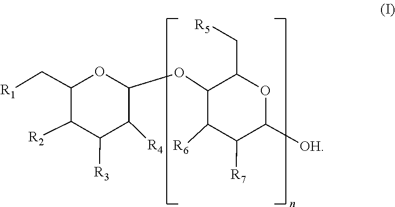
wherein
n is an integer number selected from 1-4, and
R₁, R₂, R₃, R₄, R₅, R₆, R₇ independently selected from the group consisting of —H, —OH, —NHC(O)Me, —OC(CH₃)COOH, —NH₂, and —OP(O)(OH)₂,
or a pharmaceutically acceptable salt, stereoisomeric form, or regioisomeric form thereof.

2. The Toll-Like Receptor 4 (TLR4) agonist of claim 1, wherein one or both of R₄ and R₇ are —NHC(O)Me.

3. The Toll-Like Receptor 4 (TLR4) agonist of claim 1, wherein n is 1.

4. The Toll-Like Receptor 4 (TLR4) agonist of claim 1, wherein the TLR4 agonist has a formula (II): 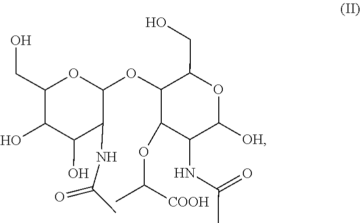 [SMILES: CC(=O)NC1C(OC2C(CO)OC(O)C(NC(C)=O)C2OC(C)C(=O)O)OC(CO)C(O)C1O]
or a pharmaceutically acceptable salt or regioisomeric form thereof.

5. The Toll-Like Receptor 4 (TLR4) agonist of claim 4, wherein the regioisomeric form of the TLR4 agonist has the formula (III):

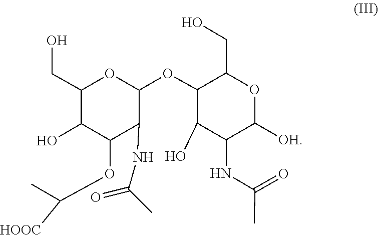 [SMILES: CC(=O)NC1C(O)OC(CO)C(OC2OC(CO)C(O)C(OC(C)C(=O)O)C2NC(C)=O)C1O]

6. The Toll-Like Receptor 4 (TLR4) agonist of claim 1, wherein the TLR4 agonist has a formula (II):

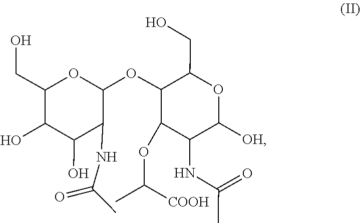 [SMILES: CC(=O)NC1C(OC2C(CO)OC(O)C(NC(C)=O)C2OC(C)C(=O)O)OC(CO)C(O)C1O]

7. The Toll-Like Receptor 4 (TLR4) agonist of claim 1, wherein the TLR4 agonist is free of any attached peptide.

8. The Toll-Like Receptor 4 (TLR4) agonist of claim 1, wherein the TLR4 agonist is synthetically derived.

9. A composition comprising the Toll-Like Receptor 4 (TLR4) agonist of claim 1.

10. The composition of claim 9, wherein the composition is a nutraceutical composition, or a supplement composition, or a nutritional composition.

11. The composition of claim 9, wherein the composition is a pharmaceutical composition.

12. The Toll-Like Receptor 4 (TLR4) agonist of claim 1 or composition of claim 9, for use as a medicament, or as an adjuvant.

13. A postbiotic comprising the Toll-Like Receptor 4 (TLR4) agonist of claim 1 or composition of claim 9.

14. An engineered probiotic microorganism capable of producing and secreting the Toll-Like Receptor 4 (TLR4) agonist of claim 1.

15. A method for promoting or maintaining gastrointestinal health in a subject; and/or modulating gut microbiota in a subject, comprising administering the Toll-Like Receptor 4 (TLR4) agonist of claim 1 or composition of claim 9 to the subject.

16. A non-therapeutic method for promoting or maintaining gastrointestinal health, or modulating gut microbiota, in a subject, comprising administering the Toll-Like Receptor 4 (TLR4) agonist of claim 1 or composition of claim 9, or postbiotic of claim 14, or engineered probiotic microorganism of claim 15 to the subject.

17. The method of claim 16, wherein the subject is a healthy subject.

18. A method for stimulating or eliciting an immune response in a subject, comprising administering the Toll-Like Receptor 4 (TLR4) agonist of claim 1 or composition of claim 9, to the subject.

19. A method for treating, preventing, or ameliorating a gastrointestinal disease in a subject, preferably the gastrointestinal disease is an inflammatory bowel disease (IBD), comprising administering the Toll-Like Receptor 4 (TLR4) agonist of claim 1 or composition of claim 9 to the subject.

20. The method of claim 19, wherein the IBD is colitis.
